(19) United States
(12) Patent Application Publication
(10) Pub. No.: US 2023/0183188 A1
(43) Pub. Date: Jun. 15, 2023

(54) ENPP1 MODULATORS AND USES THEREOF

(71) Applicant: Vir Biotechnology, Inc., San Francisco, CA (US)

(72) Inventors: Roland D. SAITO, San Mateo, CA (US); Winston C. TSE, Redwood City, CA (US)

(21) Appl. No.: 17/988,575

(22) Filed: Nov. 16, 2022

Related U.S. Application Data
(63) Continuation of application No. PCT/US2021/032249, filed on May 13, 2021.
(60) Provisional application No. 63/024,937, filed on May 14, 2020.

(51) Int. Cl. C07D 239/94; C07D 471/04; C07D 215/44; C07D 215/54; C07D 215/233; C07D 405/12; C07D 401/12; C07D 413/12; C07D 417/12

(52) U.S. Cl. CPC C07D 239/94; C07D 471/04; C07D 215/44; C07D 215/54; C07D 215/233; C07D 405/12; C07D 401/12; C07D 413/12; C07D 417/12

(57) ABSTRACT

The disclosure provides compositions and methods of quinoline, quinazolines, and pyrydylpyridines derivatives for the inhibition of ENPP1 modulators.

### CROSS-REFERENCE

This application is the by-pass continuation of International Patent Application No. PCT/US2021/032249, filed on May 13, 2021, which claims the benefit of U.S. Provisional Application No. 63/024,937, filed on May 14, 2020, the entire contents of each of which are incorporated herein by reference.

### BACKGROUND OF THE INVENTION

Ectonucleotide pyrophosphate/phosphodiesterase 1 (ENPP1) catalyzes the breakdown of extracellular adenosine triphosphate (ATP) into adenosine monophosphate (AMP) and pyrophosphate (PP)—an important inhibitor of tissue calcification. Additionally, ENPP1 degrades cyclic guanosine monophosphate-adenosine monophosphate (cGAMP), a secondary messenger molecule that mediates the upregulation of type I interferons and other inflammatory cytokines and chemokines by activating stimulator of interferon genes (STING). Therapeutically, the use of ENPP1 modulators may have particular advantage in applications such as anti-viral therapy, anti-bacterial therapy, immuno-therapy, immunological adjuvants, pyrophosphate inhibitors, and anti-inflammatory therapy.

There exists a need for new ENPP1 modulators for the treatment of disease.

### SUMMARY OF THE INVENTION

A compound represented by the structure of Formula (I):

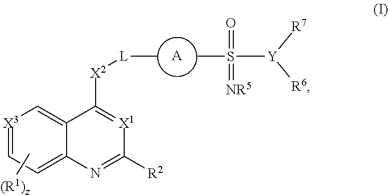

or a pharmaceutically acceptable salt thereof, wherein:
X¹ is selected from N and C(R³);
X² is absent or selected from O, S, C(R⁸)₂, N(R⁴), and C₃₋₆ carbocycle optionally substituted with one or more substituents independently selected from R⁹;
X³ is selected N and C(R^{3′});
Y is selected from N and C(H);
R¹ is selected from:
   halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, and —CN; and
   C₁₋₅ alkyl, C₂₋₅ alkenyl, C₂₋₅ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle and 3- to 6-membered heterocycle;
R² is selected from:
   hydrogen, halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, and —CN; and
   C₁₋₅ alkyl, C₂₋₅ alkenyl, C₂₋₅ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle and 3- to 6-membered heterocycle;
   R³ is selected from:
      hydrogen, halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, and —CN; and
   C₁₋₅ alkyl, C₂₋₅ alkenyl, C₂₋₅ alkynyl, each of which is optionally substituted with one or more substituents independently selected from halogen, OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle and 3- to 6-membered heterocycle;
R^{3′} is selected from:
   hydrogen, halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, and —CN; and
   C₁₋₅ alkyl, C₂₋₅ alkenyl, C₂₋₅ alkynyl, each of which is optionally substituted with one or more substituents independently selected from halogen, OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, ═O, ═S, ═N(R¹¹), —CN, C₃₋₆ carbocycle and 3- to 6-membered heterocycle;
L is absent or selected from methylene optionally substituted with one or more substituents selected from halogen, —OR²¹, —SR²¹, —N(R²¹)₂, —C(O)R²¹, —C(O)N(R²¹)₂, —N(R²¹)C(O)R²¹, —C(O)OR²¹, —OC(O)R²¹, —S(O)R²¹, —S(O)₂R²¹, —NO₂, and —CN;
Ring A is selected from an optionally substituted C₃₋₁₀ carbocycle and optionally substituted 3- to 10-membered heterocycle wherein substituents on Ring A are independently selected at each occurrence from:
   halogen, —OR³¹, —SR³¹, —N(R³¹)₂, —C(O)R³¹, —C(O)N(R³¹)₂, N(R³¹)C(O)R³¹, —C(O)OR³¹, —OC(O)R³¹, —S(O)R³¹, —S(O)₂R³¹, —NO₂, ═O, —CN, and C₁₋₅ haloalkyl; and
   C₁₋₅ alkyl C₂₋₅ alkenyl, C₂₋₅ alkynyl, each of which is optionally substituted with one or more substituents independently selected from halogen, —OR³¹, —SR³¹, —N(R³¹)₂, —C(O)R³¹, —C(O)N(R³¹)₂, —N(R³¹)C(O)R³¹, —C(O)OR³¹, —OC(O)R³¹, —S(O)R³¹, —S(O)₂R³¹, —NO₂, ═O, and —CN;
R⁴ is selected from hydrogen, C₁₋₅ alkyl, and C₃₋₆ carbocycle wherein C₁₋₅ alkyl and C₃₋₆ carbocycle are optionally substituted with one or more substituents independently selected from R⁹;
R⁵, R⁶, and R⁷ are each independently selected from hydrogen, optionally substituted C₁₋₆ alkyl, optionally substituted C₃₋₁₂ carbocycle, and optionally substituted 3- to 12-membered heterocycle; or
   R⁵ and R⁶ come together to form an optionally substituted 5- to 8-membered heterocycle, and R⁷ is selected from hydrogen, optionally substituted C₁₋₆ alkyl, optionally substituted C₃₋₁₂ carbocycle and optionally substituted 3- to 12-membered heterocycle; or
   R⁶ and R⁷ come together to form an optionally substituted 5- to 8-membered heterocycle, and R⁵ is selected from hydrogen, optionally substituted C₁₋₆ alkyl, optionally substituted C₃₋₁₂ carbocycle and optionally substituted 3- to 12-membered heterocycle;
   wherein the substituents on R⁵, R⁶, and R⁷ or rings formed therefrom are independently selected at each occurrence from:
   halogen, —OR⁴¹, —SR⁴¹, —N(R⁴¹)₂, —C(O)R⁴¹,—C(O)N(R⁴¹)₂,—N(R⁴¹)C(O)R⁴¹, —N(R⁴¹)C(O)OR⁴¹, —C(O)OR⁴¹, —OC(O)R⁴¹, —S(O)R⁴¹, —S(O)₂R⁴¹, —S(O)₂N(R⁴¹)₂, —N(R⁴¹)S(O)₂ R⁴¹, —S(O)(NR^{4′})R⁴¹, —S(NR⁴¹)₂R⁴¹, —NO₂, ═O, ═S, ═N(R⁴¹), and —CN; and
   C₁₋₅ alkyl, C₂₋₅ alkenyl, C₂₋₅ alkynyl, each of which is optionally substituted with one or more substituents independently selected from halogen, —OR⁴¹, —SR⁴¹, —N(R⁴¹)₂, —C(O)R⁴¹, —C(O)N(R⁴¹)₂, —N(R⁴¹)C(O)R⁴¹, —N(R⁴¹)C(O)OR⁴¹, —C(O)OR⁴¹, —OC(O)OR⁴¹, —S(O)R⁴¹, —S(O)₂R⁴¹, —S(O)₂N(R⁴¹)₂, —N(R⁴¹)S(O)₂R⁴¹, —S(O)(NR⁴¹)R⁴¹, —S(NR⁴¹)₂R⁴¹, —NO₂, ═O, ═S, ═N(R⁴¹), —CN, C₃₋₁₂ carbocycle and 3- to 12-membered heterocycle;
   C₃₋₁₂ carbocycle and 3- to 12-membered heterocycle each of which is optionally substituted with one or more substituents independently selected from halogen, —OR⁴¹, —SR⁴¹, —N(R⁴¹)₂, —C(O)R⁴¹, —C(O)N(R⁴¹)₂, —N(R⁴¹)C(O)R⁴¹, —N(R⁴¹)C(O)OR⁴¹, —C(O)OR⁴¹, —OC(O)R⁴¹, —S(O)R⁴¹, —S(O)₂R⁴¹, —S(O)₂N(R⁴¹)₂, —N(R⁴¹)S(O)₂R⁴¹, —S(O)(NR⁴¹)R⁴¹, —S(NR⁴¹)₂R⁴¹, —NO₂, ═O, ═S, ═N(R⁴¹), —CN, C₁₋₆ alkyl, C₁₋₆ haloalkyl, C₂₋₆ alkenyl, C₂₋₆ alkynyl, C₃₋₁₂ carbocycle and 3- to 12-membered heterocycle;
each R⁸ is independently selected from:
   hydrogen, halogen, —OR⁵¹, —SR⁵¹, —N(R⁵¹)₂, —NO₂, and —CN; and
   C₁₋₅ alkyl optionally substituted with one or more substituents independently selected from halogen, —OR⁵¹, —SR⁵¹, —N(R⁵¹)₂, —NO₂, and —CN;
each R⁹ is independently selected from:
   halogen, —OR⁶¹, —SR⁶¹, —N(R⁶¹)₂, —NO₂, and —CN; and
   C₁₋₅ alkyl optionally substituted with one or more substituents independently selected from halogen, —OR⁶¹, —SR⁶¹, —N(R⁶¹)₂, —NO₂, and —CN;
each R¹¹, R²¹, R³¹, R⁴¹, R⁵¹, and R⁶¹ is independently selected from: hydrogen; and C₁₋₅ alkyl, C₂₋₅ alkenyl, C₂₋₅ alkynyl, C₃₋₈ carbocycle, and 3- to 8-membered heterocycle, each of which is optionally substituted with one or more substituents independently selected from halogen, —OH, —CN, —NO₂, —NH₂, ═O, ═S, C₁₋₅ alkyl, —C₁₋₅ haloalkyl, and —O—C₁₋₅ alkyl; and z is selected from 0-3.

In certain embodiments, the compound or salt of Formula (I) is represented by Formula (IA), (IB), (IC), (ID), and (IE):

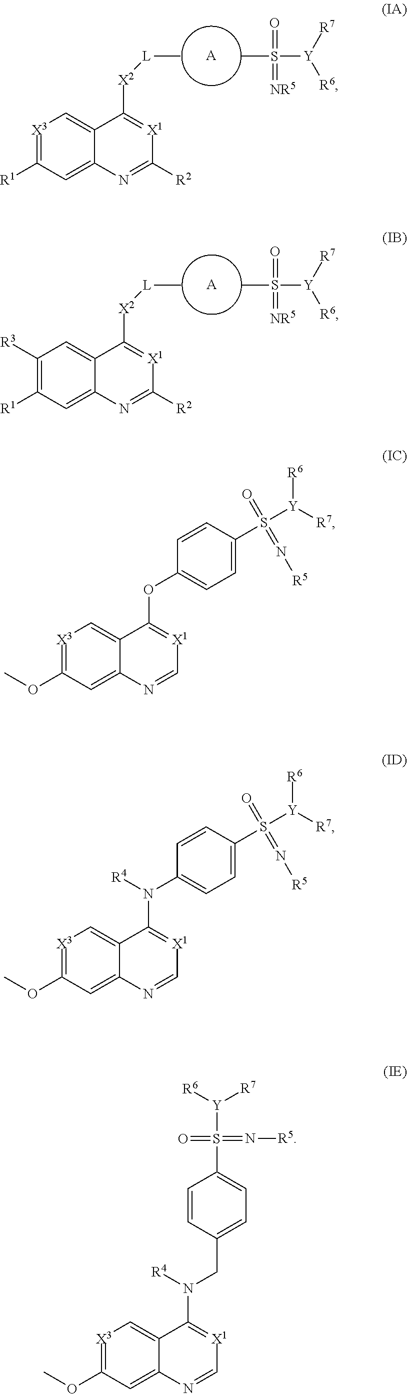

In certain embodiments, the disclosure provides a pharmaceutical composition comprising the compound or salt of Formula (IA), (IB), (IC), (ID), or (IE), and a pharmaceutically acceptable excipient.

In certain embodiments, the disclosure provides a method of inhibiting ENPP1 in a subject in need thereof, comprising administering to the subject a compound or salt of Formula (IA), (IB), (IC), (ID), or (IE).

In certain embodiments, the disclosure provides a method of activating STING activity in a subject in need thereof, comprising administering to the subject a compound of Formula (IA), (IB), (IC), (ID), or (IE).

In certain embodiments, the disclosure provides a method of activating an immune response to a pathogen in a subject in need thereof, comprising administering to the subject a compound of Formula (IA), (IB), (IC), (ID), or (IE).

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to saturated hydrocarbon groups, including straight-chain alkyl and branched-chain alkyl groups that contain from 1 to 6 carbons. For example —C₁₋₆ alkyl- may be selected from methyl, ethyl, propyl, butyl, pentyl, and hexyl, any one of which is optionally substituted. The term —C_{x-y}alkylene- refers to a substituted or unsubstituted alkylene chain with from x to y carbons in the alkylene chain. For example —C₁₋₆ alkylene- may be selected from methylene, ethylene, propylene, butylene, pentylene, and hexylene, any one of which is optionally substituted.

“Alkyl” as used herein refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tent-butyl), 1-pentyl (n-pentyl). The alkyl is attached to the rest of the molecule by a single bond.

“Alkenyl” as used herein refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” as used herein refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

The terms “C_{x-y}alkenyl” and “C_{x-y}alkynyl” as used herein refer to substituted or unsubstituted unsaturated aliphatic groups analogous in length and possible substitution to the alkyls described above, but that contain at least one double or triple bond, respectively. The term —C_{x-y}alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkenylene chain. For example, —C₂₋₆alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” as used herein refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. Alkylene chain may be optionally substituted by one or more substituents such as those substituents described herein.

“Alkenylene” as used herein refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. Alkenylene chain may be optionally substituted by one or more substituents such as those substituents described herein.

“Alkynylene” as used herein refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. Alkynylene chain may be optionally substituted by one or more substituents such as those substituents described herein.

“Halo” or “halogen” as used herein refers to halogen substituents such as bromo, chloro, fluoro and iodo substituents.

“Haloalkyl” as used herein refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. Examples of halogen substituted alkanes (“haloalkanes”) include halomethane (e.g., chloromethane, bromomethane, fluoromethane, iodomethane), di-and trihalomethane (e.g., trichloromethane, tribromomethane, trifluoromethane, triiodomethane), 1-haloethane, 2-haloethane, 1,2-dihaloethane, and any other suitable combinations of alkanes (or substituted alkanes) and halogens. When an alkyl group is substituted with more than one halogen radicals, each halogen may be independently selected, for example 1-chloro,2-bromoethane.

“Carbocycle” as used herein refers to a saturated, unsaturated or aromatic ring in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Carbocycle may be optionally substituted by one or more substituents such as those substituents described herein. Bicyclic carbocycles may be fused, bridged or spiro-ring systems.

“Aryl” as used herein refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

“Heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. In an exemplary embodiment, a heterocycle, e.g., pyridyl, may be fused to a saturated or unsaturated ring, e.g., cyclohexane, cyclopentane, or cyclohexene. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Heterocycle may be optionally substituted by one or more substituents such as those substituents described herein. Bicyclic heterocycles may be fused, bridged or spiro-ring systems.

“Heteroaryl” includes aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more rings in which two or more atoms are common to two adjoining rings wherein at least one of the rings is heteroaromatic, e.g., the other rings can be aromatic or non-aromatic carbocyclic, or heterocyclic. Heteroaryl groups include, for example, pyrrole, furan, thiophene, imidazole, oxazole, thiazole, pyrazole, pyridine, pyrazine, pyridazine, and pyrimidine, and the like.

“Substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example. halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain. It will be understood by those skilled in the art that substituents can themselves be substituted, if appropriate.

A pharmaceutically acceptable salt also refers to any salt which may form in vivo as a result of administration of an acid, another salt, or a prodrug which is converted into an acid or salt. A salt comprises one or more ionic forms of the compound, such as a conjugate acid or base, associated with one or more corresponding counterions. Salts can form from or incorporate one or more deprotonated acidic groups (e.g. carboxylic acids), one or more protonated basic groups (e.g. amines), or both (e.g. zwitterions).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “subject,” “individual,” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms ““administer”, “administered”, “administers” and “administering” a compound should be understood to mean providing a compound of the invention or a prodrug of a compound of the invention to the individual in need.

The term “effective amount” or “therapeutically effective amount” refers to that amount of a compound or salt described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term can also apply to a dose that can induce a particular response in target cells, e.g., reduction of proliferation or down regulation of activity of a target protein. The specific dose can vary depending on the particular compounds chosen, the dosing regimen to be followed, whether it is administered in combination with other compounds, timing of administration, the tissue to which it is administered, and the physical delivery system in which it is carried.

“Treatment” or “treating” as used herein refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “inhibit”, “selective inhibition” or “selectively inhibit” as referred to a biologically active agent refers to the agent's ability to preferentially reduce the target signaling activity as compared to off-target signaling activity, via direct or interact interaction with the target.

### Compounds

In some aspects, the present disclosure provides a compound represented by the structure of Formula (I):

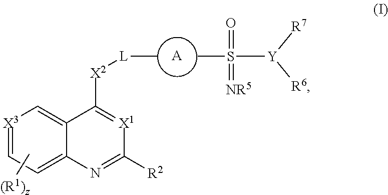

or a pharmaceutically acceptable salt thereof, wherein:
X¹ is selected from N and C(R³);
X² is absent or selected from O, S, C(R⁸)₂, N(R⁴), and C₃₋₆ carbocycle optionally substituted with one or more substituents independently selected from R⁹;
X³ is selected N and C(R^{3′});
Y is selected from N and C(H);
R¹ is selected from:
   halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, and —CN; and
   C₁₋₅ alkyl, C₂₋₅ alkenyl, C₂₋₅ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle and 3- to 6-membered heterocycle;
R² is selected from:
   hydrogen, halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, and —CN; and
   C₁₋₅ alkyl, C₂₋₅ alkenyl, C₂₋₅ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle and 3- to 6-membered heterocycle;
R³ is selected from:
   hydrogen, halogen, —OR¹¹, —SR¹¹, —N(R¹¹)₂, —C(O)R¹¹,—C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, and —CN; and
   C₁₋₅ alkyl, C₂₋₅ alkenyl, C₂₋₅ alkynyl, each of which is optionally substituted with one or more substituents independently selected from halogen, OR¹¹, —SR¹¹, —N(R¹¹)₂, —C(O)R¹¹, —C(O)N(R¹¹)₂,—N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, ═O, ═S, ═N(R¹¹), —CN, C₃₋₆ carbocycle and 3- to 6-membered heterocycle;
R^{3′} is selected from:
   hydrogen, halogen, —OR¹¹, —SR¹¹, —N(R¹¹)₂, —C(O)R¹¹,—C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, and —CN; and
   C₁₋₅ alkyl, C₂₋₅ alkenyl, C₂₋₅ alkynyl, each of which is optionally substituted with one or more substituents independently selected from halogen, OR¹¹, —SR¹¹, —N(R¹¹)₂, —C(O)R¹¹, —C(O)N(R¹¹)₂,—N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, ═O, ═S, ═N(R¹¹), —CN, C₃₋₆ carbocycle and 3- to 6-membered heterocycle;
L is absent or selected from methylene optionally substituted with one or more substituents selected from halogen, —OR²¹, —SR²¹, —N(R²¹)₂, —C(O)R²¹, —C(O)N(R²¹)₂, —N(R²¹)C(O)R²¹, —C(O)OR²¹, —OC(O)R²¹, —S(O)R²¹, —S(O)₂R²¹, —NO₂, and —CN;
Ring A is selected from an optionally substituted C₃₋₁₀ carbocycle and optionally substituted 3- to 10-membered heterocycle wherein substituents on Ring A are independently selected at each occurrence from:
   halogen, —OR³¹, —SR³¹, —N(R³¹)₂, —C(O)R³¹, —C(O)N(R³¹)₂, N(R³¹)C(O)R³¹, —C(O)OR³¹, —OC(O)R³¹, —S(O)R³¹, —S(O)₂R³¹, —NO₂, ═O, —CN, and C₁₋₅ haloalkyl; and
   C₁₋₅ alkyl C₂₋₅ alkenyl, C₂₋₅ alkynyl, each of which is optionally substituted with one or more substituents independently selected from halogen, —OR³¹, —SR³¹, —N(R³¹)₂, —C(O)R³¹, —C(O)N(R³¹)₂, —N(R³¹)C(O)R³¹, —C(O)OR³¹, —OC(O)R³¹, —S(O)R³¹, —S(O)₂R³¹, —NO₂, ═O, and —CN;
R⁴ is selected from hydrogen, C₁₋₅ alkyl, and C₃₋₆ carbocycle wherein C₁₋₅ alkyl and C₃₋₆ carbocycle are optionally substituted with one or more substituents independently selected from R⁹;
R⁵, R⁶, and R⁷ are each independently selected from hydrogen, optionally substituted C₁₋₆ alkyl, optionally substituted C₃₋₁₂ carbocycle, and optionally substituted 3- to 12-membered heterocycle; or
   R⁵ and R⁶ come together to form an optionally substituted 5- to 8-membered heterocycle, and R⁷ is selected from hydrogen, optionally substituted C₁₋₆ alkyl, optionally substituted C₃₋₁₂ carbocycle and optionally substituted 3- to 12-membered heterocycle; or
   R⁶ and R⁷ come together to form an optionally substituted 5- to 8-membered heterocycle, and R⁵ is selected from hydrogen, optionally substituted C₁₋₆ alkyl, optionally substituted C₃₋₁₂ carbocycle and optionally substituted 3- to 12-membered heterocycle;
   wherein the substituents on R⁵, R⁶, and R⁷ or rings formed therefrom are independently selected at each occurrence from:
   halogen, —OR⁴¹, —SR⁴¹, —N(R⁴¹)₂, —C(O)R⁴¹,—C(O)N(R⁴¹)₂,—N(R⁴¹)C(O)R⁴¹, —N(R⁴¹)C(O)OR⁴¹, —C(O)OR⁴¹, —C(O)R⁴¹, —S(O)R⁴¹, —S(O)₂R⁴¹, —S(O)₂N(R⁴¹)², S(O)₂N(R⁴¹)₂, —N(R⁴¹)S(O)₂ R⁴¹, —S(O)(NR^{4′})R⁴¹, —S(NR⁴¹)₂R⁴¹, —NO₂, ═O, ═S, ═N(R⁴¹), and —CN; and
   C₁₋₅ alkyl, C₂₋₅ alkenyl, C₂₋₅ alkynyl, each of which is optionally substituted with one or more substituents independently selected from halogen, —OR⁴¹, —SR⁴¹, —N(R⁴¹)₂, —C(O)R⁴¹, —C(O)N(R⁴¹)₂, —N(R⁴¹)C(O)R⁴¹, —N(R⁴¹)C(O)OR⁴¹, —C(O)OR⁴¹, —OC(O)R⁴¹, —S(O)R⁴¹, —S(O)₂R⁴¹, —S(O)₂N(R⁴¹)₂, —N(R⁴¹)S(O)₂R⁴¹, —S(O)(NR⁴¹)R⁴¹, —S(NR⁴¹)₂R⁴¹, —NO₂, ═O, ═S, ═N(R⁴¹), —CN, C₃₋₁₂ carbocycle and 3- to 12-membered heterocycle;
   C₃₋₁₂ carbocycle and 3- to 12-membered heterocycle each of which is optionally substituted with one or more substituents independently selected from halogen, —OR⁴¹, —SR⁴¹, —N(R⁴¹)₂, —C(O)R⁴¹, —C(O)N(R⁴¹)₂, —N(R⁴¹)C(O)R⁴¹, —N(R⁴¹)C(O)OR⁴¹, —C(O)OR⁴¹, —OC(O)R⁴¹, —S(O)R⁴¹, —S(O)₂R⁴¹, —S(O)₂N(R⁴¹)₂, —N(R⁴¹)S(O)₂R⁴¹, —S(O)(NR⁴¹)R⁴¹, —S(NR⁴¹)₂R⁴¹, —NO₂, ═O, ═S, ═N(R⁴¹), —CN, C₁₋₆ alkyl, C₁₋₆ haloalkyl, C₂₋₆ alkenyl, C₂₋₆ alkynyl, C₃₋₁₂ carbocycle and 3- to 12-membered heterocycle;
each R⁸ is independently selected from:
   hydrogen, halogen, —OR⁵¹, —SR⁵¹, —N(R⁵¹)₂, —NO₂, and —CN; and
   C₁₋₅ alkyl optionally substituted with one or more substituents independently selected from halogen, —OR⁵¹, —SR⁵¹, —N(R⁵¹)₂, —NO₂, and —CN;
each R⁹ is independently selected from:
   halogen, —OR⁶¹, —SR⁶¹, —N(R⁶¹)₂, —NO₂, and —CN; and
   C₁₋₅ alkyl optionally substituted with one or more substituents independently selected from halogen, —OR⁶¹, —SR⁶¹, —N(R⁶¹)₂, —NO₂, and —CN;
each R¹¹, R²¹, R³¹, R⁴¹, R⁵¹, and R⁶¹ is independently selected from: hydrogen; and C₁₋₅ alkyl, C₂₋₅ alkenyl, C₂₋₅ alkynyl, C₃₋₈ carbocycle, and 3- to 8-membered heterocycle, each of which is optionally substituted with one or more substituents independently selected from halogen, —OH, —CN, —NO₂, —NH₂, ═O, ═S, C₁₋₅ alkyl, —C₁₋₅ haloalkyl, and —O—C₁₋₅ alkyl; and z is selected from 0-3.

In some embodiments, for the compound or salt of Formula (I), X¹ is selected from C(R³) and N. In some embodiments, X¹ is C(R³). In some embodiments, R³ of C(R³) is selected from hydrogen, halogen, —OR¹¹, —N(R¹¹)₂, —NO₂, and —CN; and C₁₋₅ alkyl optionally substituted with one or more substituents independently selected from halogen, OR¹¹, N(R¹¹), —CN, C₃₋₆ carbocycle and 3- to 6-membered heterocycle. In some embodiments, R³ of C(R³) is selected from hydrogen, halogen, and —CN. In some embodiments, X¹ is C(CN). In some embodiments, X¹ is C(H). In some embodiments, X¹ is N.

In some embodiments, for the compound or salt of Formula (I), X² is selected from O, S, C(R⁸)₂, N(R⁴), and C₃₋₆ carbocycle optionally substituted with one or more substituents independently selected from R⁹. In some embodiments, X² is S. In some embodiments, X² is C₃₋₆ carbocycle optionally substituted with one or more substituents independently selected from R⁹. In some embodiments, X² is O or N(R⁴). In some embodiments, X² is O. In some embodiments, X² is N(H).

In some embodiments, for the compound or salt of Formula (I), R¹ is independently selected from halogen, —OR¹¹, —SR¹¹, —N(R¹¹)₂, —C(O)N(R¹¹)₂, N(R¹¹)C(O)R¹¹, —C(O)OR¹¹, —OC(O)R¹¹, —NO₂, —CN and C₁₋₅ alkyl optionally substituted with one or more substituents independently selected from halogen, —OR¹¹, —CN and —NO₂. In some embodiments, R¹ is independently selected from halogen, —OCH₃, —NO₂, —CN and C₁₋₅ alkyl optionally substituted with one or more substituents independently selected from halogen, —OCH₃, —CN and —NO₂. In some embodiments, R¹ is —OCH₃.

In some embodiments, for the compound or salt of Formula (I), z is selected from 0-3. In some embodiments, z is selected from 1-3. In some embodiments, z is selected from 1, 2 and 3. In some embodiments, z is 3. In some embodiments, z is 2. In some embodiments, z is 1. In some embodiments, X¹ is C(H) and z is 0. In some embodiments, X¹ is C(H) and z is 1.

In some embodiments, Formula (I) is represented by Formula (IA):

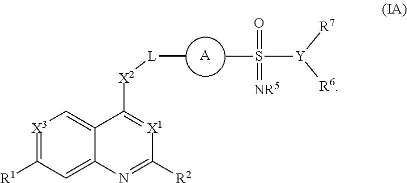

In some embodiments, for the compound or salt of Formula (I), X³ is selected from C(R^{3′}) and N. In some embodiments, X³ is selected from C(H) and N. In some embodiments, X³ is N. In some embodiments, X³ is C(R^{3′}). In some embodiments, R^{3′} of C(R^{3′}) selected from hydrogen, halogen, OR¹¹ and —CN. In some embodiments, C(R^{3′}) of X³ is selected from C(F), C(F), and C(Br). In some embodiments, C(R^{3′}) of X³ is selected from C(H), C(OR¹¹), and C(CN). In some embodiments C(R^{3′}) of X³ is selected from C(H) and C(OR¹¹). In some embodiments C(R^{3′}) of X³ is C(H). In some embodiments C(R^{3′}) of X³ is C(OCH₃).

In some embodiments, Formula (I) is represented by Formula (IB):

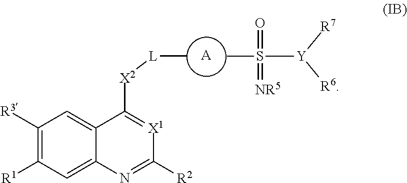

In some embodiments, for the compound or salt of Formula (I) and (IB), z is 2 and each R¹ are independently selected from halogen, —OR¹¹, —SR¹¹, —N(R¹¹)₂, —C(O)N(R¹¹)₂, N(R¹¹)C(O)R¹¹, —C(O)OR¹¹, —OC(O)R¹¹, —NO₂, —CN and C₁₋₃ alkyl optionally substituted with one or more substituents independently selected from halogen, —OR¹¹, —CN and —NO₂. In some embodiments, z is 2 and each R¹ are independently selected from halogen and —OR¹¹. In some embodiments, z is 2; R¹ are each independently selected from halogen and —OR¹¹; and X¹ is selected from C(R³). In some embodiments, z is 1 and each R¹ are independently selected from halogen and —OR¹¹. In some embodiments, z is 1; R¹ are each independently selected from halogen and —OR¹¹; and X¹ is selected from C(R³).

In some embodiments, for the compound or salt of Formula (I), (IA), and (IB), R² is selected from hydrogen, halogen, —OR¹¹, —SR¹¹, —N(R¹¹)₂, —C(O)R¹¹, and —CN; and C₁₋₅ alkyl, each of which is optionally substituted with one or more substituents independently selected from halogen, —OR¹¹, —N(R¹¹)₂, —C(O)R¹¹, —C(O)N(R¹¹)₂, —S(O)₂R¹¹, —S(O)₂N(R¹¹)₂, —═O, ═S, ═N(R¹¹), —CN, C₃₋₆ carbocycle and 3- to 6-membered heterocycle. In some embodiments, R² is selected from hydrogen, halogen, —OR¹¹, —SR¹¹, —N(R¹¹)₂, —C(O)R¹¹, and —CN; and optionally substituted C₁₋₃ alkyl. In some embodiments, R² is selected from hydrogen, halogen, - and —CN. In some embodiments, R² is hydrogen.

In some embodiments, for the compound or salt of Formula (I), (IA), and (IB), L is absent or optionally substituted methylene. In some embodiments, L is methylene optionally substituted with one or more substituents selected from halogen, —OR²¹, —N(R²¹)₂, —C(O)R²¹, —C(O)N(R²¹)², N(R²¹)C(O)R²¹, —C(O)OR²¹, —OC(O)R²¹, —S(O)R²¹, —S(O)₂R²¹, —NO₂, and —CN. In some embodiments, L is methylene optionally substituted with one or more substituents selected from halogen and —OR²¹. In some embodiments, for the compound or salt of Formula (I), (IA), and (TB), L is methylene. In some embodiments, L is absent.

In some embodiments, for the compound or salt of Formula (I), (IA), and (IB), L is absent and R² is hydrogen. In some embodiments, L is methylene and R² is hydrogen.

In some embodiments, for the compound or salt of Formula (I), (IA), and (IB), Ring A is selected from optionally substituted C₆₋₁₀ carbocycle and optionally substituted 3- to 10-membered heterocycle wherein substituents on optionally substituted C₆₋₁₀ carbocycle and optionally substituted 3- to 10-membered heterocycle are independently selected at each occurrence from: halogen, —OR³¹, —N(R³¹)₂, —C(O)R³¹, —C(O)N(R³¹)₂, N(R³¹)C(O)R³¹, —C(O)OR³¹, —OC(O)R³¹, —NO₂, —CN, and C₁₋₅ haloalkyl; and
C₁₋₅ alkyl optionally substituted with one or more substituents independently selected from halogen, —OR³¹, —NO₂, ═O, and —CN.

In some embodiments, for the compound or salt of Formula (I), (IA), and (IB), Ring A is an optionally substituted C₃₋₁₀ carbocycle. In some embodiments, the optionally substituted C₃₋₁₀ carbocycle of Ring A is saturated. In some embodiments, the optionally substituted C₃₋₁₀ carbocycle of Ring A is unsaturated. In some embodiments, Ring A is an optionally substituted C₃₋₆ carbocycle. In some embodiments, Ring A is an optionally substituted C₆₋₁₀ carbocycle. In some embodiments, the C₃₋₁₀ carbocycle of Ring A is selected from optionally substituted C₃ carbocycle, optionally substituted C₄ carbocycle, optionally substituted C₅ carbocycle, optionally substituted C₆ carbocycle, optionally substituted C₇ carbocycle, optionally substituted C₈ carbocycle, and optionally substituted C₉ carbocycle. In some embodiments, the optionally substituted C₃₋₁₀ carbocycle of Ring A is an optionally substituted C₆ carbocycle.

In some embodiments, for the compound or salt of Formula (I), (IA), and (IB), Ring A is an optionally substituted 3- to 10-membered heterocycle. In some embodiments, the optionally substituted 3- to 10-membered heterocycle of Ring A is saturated. In some embodiments, the optionally substituted 3- to 10-membered heterocycle of Ring A is unsaturated. In some embodiments, the optionally substituted 3- to 10-membered heterocycle of Ring A is selected from optionally substituted 3-membered heterocycle, optionally substituted 4-membered heterocycle, optionally substituted 5-membered heterocycle, optionally substituted 6-membered heterocycle, optionally substituted 7-membered heterocycle, optionally substituted 8-membered heterocycle, optionally substituted 9-membered heterocycle, and optionally substituted 10-membered heterocycle. In some embodiments, the optionally substituted 3- to 10-membered heterocycle of Ring A is an optionally substituted 6-membered heterocycle.

In some embodiments, for the compound or salt of Formula (I), (IA), and (IB), Ring A is an optionally substituted 3- to 10-membered heterocycle comprising at least one heteroatom selected from oxygen, nitrogen, sulfur, or any combination thereof. In some embodiments, Ring A is an optionally substituted 3- to 10-membered heterocycle comprising at least one heteroatom selected from oxygen, nitrogen, or any combination thereof. In some embodiments, Ring A is an optionally substituted 3- to 10-membered heterocycle comprising at least one heteroatom is oxygen. In some embodiments, Ring A is an optionally substituted 3- to 10-membered heterocycle comprising at least one heteroatom is nitrogen.

In some embodiments, for the compound or salt of Formula (I), (IA), and (IB), Ring A is selected from an optionally substituted C₃₋₆ carbocycle and optionally substituted 3- to 6-membered heterocycle. In some embodiments, Ring A is selected from an optionally substituted C₆ carbocycle and optionally substituted 6-membered heterocycle.

In some embodiments, for the compound or salt of Formula (I), (IA), and (IB), Ring A is selected from an optionally substituted aryl and optionally substituted heteroaryl wherein substituents on Ring A are independently selected at each occurrence from: halogen, —OR³¹, —N(R³¹)₂, —C(O)R³¹, —C(O)N(R³¹)₂, N(R³¹)C(O)R³¹, —C(O)OR³¹, —OC(O)R³¹, —NO₂, —CN, and C₁₋₅ haloalkyl; and

C₁₋₅ alkyl optionally substituted with one or more substituents independently selected from halogen, —OR³¹, —NO₂, ═O, and —CN.

In some embodiments, for the compound or salt of Formula (I), (IA), and (IB), Ring A is selected from an optionally substituted aryl and optionally substituted 6-membered heteroaryl, wherein substituents on Ring A are independently selected at each occurrence from: halogen, —OR³¹, —N(R³¹)₂, —C(O)R³¹, —C(O)N(R³¹)₂, N(R³¹)C(O)R³¹, —C(O)OR³¹, —OC(O)R³¹, —NO₂, —CN, and C₁₋₅ haloalkyl; and

C₁₋₅ alkyl optionally substituted with one or more substituents independently selected from halogen, —OR³¹, —NO₂, ═O, and —CN.

In some embodiments, for the compound or salt of Formula (I), (IA), and (IB), Ring A is selected from optionally substituted phenyl and optionally substituted 6-membered heteroaryl. In some embodiments, Ring A is selected from optionally substituted phenyl and optionally substituted 6-membered heteroaryl wherein substituents on each ring are independently selected at each occurrence from from halogen, —OR³¹, —NO₂ and optionally substituted C₁₋₅ alkyl. In some embodiments, Ring A is an optionally substituted phenyl or optionally substituted pyridyl. In some embodiments, Ring A is represented by

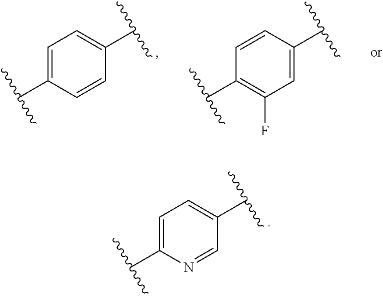 [SMILES: *c1ccc(*)cc1 ; *c1ccc(*)c(F)c1 ; *c1ccc(*)nc1]

In some embodiments, Ring A is

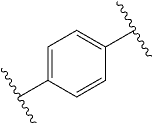 [SMILES: *c1ccc(*)cc1]

In some embodiments, Ring A is

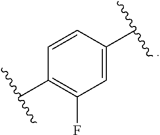 [SMILES: *c1ccc(*)c(F)c1]

In some embodiments, Ring A is

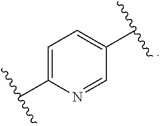 [SMILES: *c1ccc(*)nc1]

In some embodiments, for the compound or salt of Formula (I), the compound is selected from Formula (IC), (ID), and (IE):

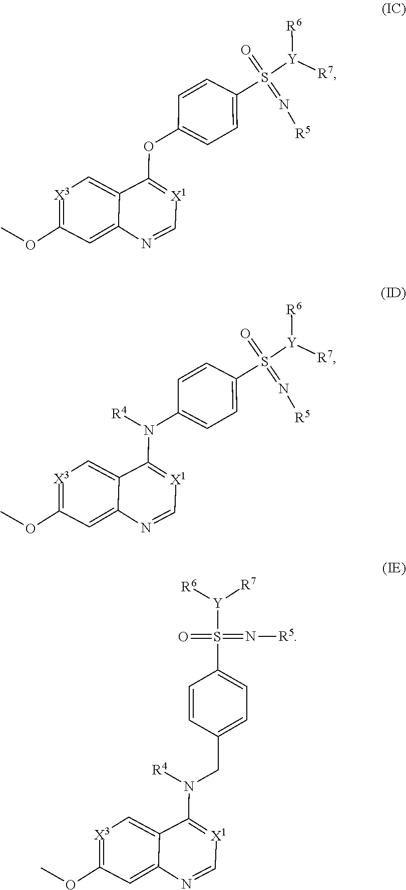

In some embodiments, for the compound or salt of Formula (I), the compound is represented by Formula (IC):

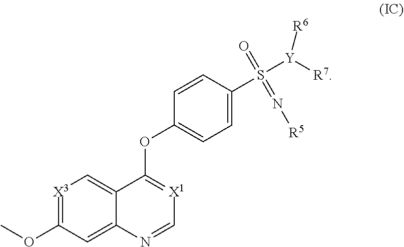

In some embodiments, for the compound or salt of Formula (I), the compound is represented by Formula (ID):

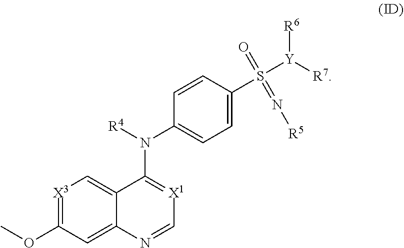

In some embodiments, for the compound or salt of Formula (I), the compound is represented by Formula (IE):

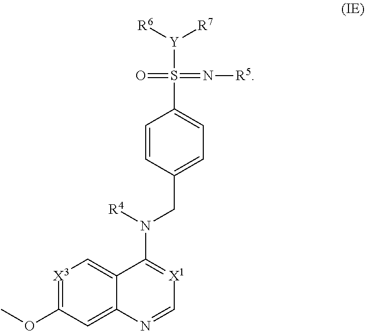

In some embodiments, for the compound or salt of Formula (I), (IA), (IB), (IC), (ID), and (IE), X¹ is N.

In some embodiments, for the compound or salt of Formula (I), (IA), (IB), (IC), (ID), and (IE), X³ is selected from C(R^{3′}) and N. In some embodiments, X³ is selected from C(H) and N. In some embodiments, X³ is N. In some embodiments, X³ is C(R^{3′}). In some embodiments, R^{3′} of C(R^{3′}) selected from hydrogen, halogen, OR¹¹ and —CN. In some embodiments, C(R^{3′}) of X³ is selected from C(F), C(F), and C(Br). In some embodiments, C(R^{3′}) of X³ is selected from C(H), C(OR¹¹), and C(CN). In some embodiments C(R^{3′}) of X³ is selected from C(H) and C(OR¹¹). In some embodiments C(R^{3′}) of X³ is C(OCH₃). In some embodiments C(R^{3′}) of X³ is C(H).

In some embodiments, for the compound or salt of Formula (I), (IA), (IB), (IC), (ID), and (IE), Y is selected from N and C(H). In some embodiments, Y is N. In some embodiments, Y is C(H). In some embodiments, Y is is C(H) and both R⁶ and R⁷ are independently selected from hydrogen, optionally substituted C₁₋₆ alkyl, optionally substituted C₃₋₆ carbocycle and optionally substituted 3- to 6-membered heterocycle. In some embodiments, Y is C(H) and both R⁶ and R⁷ are independently selected from hydrogen and optionally substituted C₁₋₆alkyl. In some embodiments, Y is C(H), R⁶ is hydrogen, and R⁷ is optionally substituted C₁₋₆ alkyl.

In some embodiments, for the compound or salt of Formula (I), (IA), (IB), (IC), (ID), and (IE), R⁵, R⁶, and R⁷ are each independently selected from hydrogen, optionally substituted C₁₋₆ alkyl, optionally substituted C₃₋₁₂ carbocycle and optionally substituted 3- to 12-membered heterocycle, wherein substituents on C₁₋₆ alkyl, C₃₋₁₂ carbocycle and 3- to 12-membered heterocycle are independently selected at each occurrence from halogen, —OR⁴¹, —N(R⁴¹)₂, —C(O)R⁴¹, —C(O)N(R⁴¹)₂, —N(R⁴¹)C(O)R⁴¹, —C(O)OR⁴¹, —OC(O)R⁴¹, —NO₂, ═O, and —CN; and

C₁₋₅ alkyl optionally substituted with one or more substituents independently selected from halogen, —OR⁴¹, —N(R⁴¹)₂, —NO₂, ═O, and —CN; and

phenyl or 6-membered heteroaryl, each of which is optionally substituted with one or more substituents independently selected from halogen, —OR⁴¹, —N(R⁴¹)₂, —NO₂, ═O, ═S, —CN, C₁₋₆ alkyl, C₁₋₆ haloalkyl, C₂₋₆ alkenyl, C₂₋₆ alkynyl, phenyl and 6-membered heteroaryl.

In some embodiments, for the compound or salt of Formula (I), (IA), (IB), (IC), (ID), and (IE), R⁵, R⁶, and R⁷ are each hydrogen.

In some embodiments, for the compound or salt of Formula (I), (IA), (IB), (IC), (ID), and (IE), R⁵, R⁶ and R⁷ are each independently selected from optionally substituted C₁₋₆ alkyl, optionally substituted C₃₋₁₂ carbocycle and optionally substituted 3- to 12-membered heterocycle.

In some embodiments, for the compound or salt of Formula (I), (IA), (IB), (IC), (ID), and (IE), two of R⁵, R⁶ and R⁷ are hydrogen and the other of R⁵, R⁶ and R⁷ is selected from hydrogen, optionally substituted C₁₋₆ alkyl, optionally substituted C₃₋₁₂ carbocycle and optionally substituted 3- to 12-membered heterocycle. In some embodiments, R⁵ and R⁶ are each hydrogen. In some embodiments, R⁶ and R⁷ are each hydrogen.

In some embodiments, for the compound or salt of Formula (I), (IA), (IB), (IC), (ID), and (IE), R⁵ and R⁶ are each hydrogen and R⁷ is selected from hydrogen, optionally substituted C₁₋₆ alkyl, optionally substituted C₃₋₁₂ carbocycle and optionally substituted 3- to 12-membered heterocycle. In some embodiments, R⁵ and R⁶ are each hydrogen and R⁷ is selected from optionally substituted C₁₋₄ alkyl, optionally substituted C₃₋₁₀ carbocycle and optionally substituted 3- to 10-membered heterocycle. In some embodiments, R⁵ and R⁶ are each hydrogen and R⁷ is C₁₋₄ alkyl optionally substituted with one or more substituents selected from halogen, —OR⁴¹, —N(R⁴¹)₂, —C(O)R⁴¹, —C(O)N(R⁴¹)₂, —N(R⁴¹)C(O)R⁴¹, —N(R⁴¹)C(O)OR⁴¹, —C(O)OR⁴¹, ═O, and —CN, C₃₋₁₀ carbocycle and 3- to 10-membered heterocycle; and wherein C₃₋₁₀ carbocycle and 3- to 10-membered heterocycle each of which is optionally substituted with one or more substituents independently selected from halogen, —OR⁴¹, —N(R⁴¹)₂, —C(O)R⁴¹, —C(O)OR⁴¹, —NO₂, —CN, C₁₋₆ alkyl, C₁₋₆ haloalkyl, C₂₋₆ alkenyl, C₂₋₆ alkynyl, C₃₋₁₂ carbocycle and 3- to 12-membered heterocycle.

In some embodiments, for the compound or salt of Formula (I), (IA), (IB), (IC), (ID), and (IE), R⁵ and R⁶ are each hydrogen and R⁷ is C₁₋₄ alkyl optionally substituted with one or more substituents selected from halogen, —OR⁴¹, —C(O)R⁴¹, —C(O)OR⁴¹ and —CN. In some embodiments, R⁷ is C₁₋₄ alkyl optionally substituted with one or more substituents selected from halogen, —OR⁴¹ wherein R⁴¹ is selected from hydrogen and C₁₋₅ alkyl optionally substituted with one or more substituents selected from halogen, —OH, —CN, and —NO_{2.} In some embodiments, R⁷ is C₁₋₄ alkyl optionally substituted with one or more substituents selected from —OR⁴¹ wherein R⁴¹ is selected from hydrogen and C₁₋₅ alkyl optionally substituted with one or more substituents selected from halogen and —OH. In some embodiments, R⁵ and R⁶ are each hydrogen and R⁷ is selected from

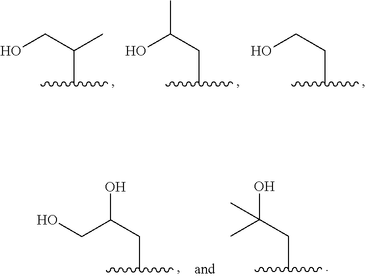

In some embodiments, for the compound or salt of Formula (I), (IA), (IB), (IC), (ID), and (IE), R⁵ and R⁶ are each hydrogen and R⁷ is C₁₋₄ alkyl optionally substituted with one or more substituents selected —OR⁴¹ and C₃₋₆ carbocycle wherein the C₃₋₆ carbocycle is optionally substituted with one or more substituents independently selected from halogen, —OR⁴¹, —NO₂, —CN, C₁₋₆ alkyl, and C₁₋₆ haloalkyl. In some embodiments, R⁵ and R⁶ are each hydrogen and R⁷ is C₁₋₄ alkyl is substituted with one or more substituents selected —OR⁴¹ wherein R⁴¹ is selected from hydrogen and C₁₋₅ alkyl; and C₃₋₆ carbocycle wherein C₃₋₆ carbocycle is optionally substituted with one or more substituents independently selected from halogen, —OR⁴¹, —NO₂, —CN, C₁₋₃ alkyl, C₁₋₃ haloalkyl. In some embodiments, R⁵ and R⁶ are each hydrogen and R⁷ is

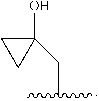 [SMILES: *CC1(O)CC1]

In some embodiments, for the compound or salt of Formula (I), (IA), (IB), (IC), (ID), and (IE), R⁶ and R⁷ are each hydrogen and R⁵ is selected from hydrogen, optionally substituted C₁₋₆ alkyl, optionally substituted C₃₋₁₂ carbocycle and optionally substituted 3- to 12-membered heterocycle. In some embodiments, R⁶ and R⁷ are each hydrogen and R⁵ is selected from optionally substituted C₁₋₄ alkyl, optionally substituted C₃₋₁₀ carbocycle and optionally substituted 3- to 10-membered heterocycle. In some embodiments, R⁶ and R⁷ are each hydrogen and R⁵ is C₁₋₄ alkyl optionally substituted with one or more substituents selected from halogen, —OR¹¹, —N(R⁴¹)₂, —C(O)R⁴¹, —C(O)N(R⁴¹)₂, —N(R⁴¹)C(O)R⁴¹, —N(R⁴¹)C(O)OR⁴¹, —C(O)OR⁴¹, ═O, and —CN, C₃₋₁₀ carbocycle and 3- to 10-membered heterocycle; and wherein C₃₋₁₀ carbocycle and 3- to 10-membered heterocycle each of which is optionally substituted with one or more substituents independently selected from halogen, —OR⁴¹, —N(R⁴¹)₂, —C(O)R⁴¹, —C(O)OR⁴¹, —NO₂, —CN, C₁₋₆ alkyl, C₁₋₆ haloalkyl, C₂₋₆ alkenyl, C₂₋₆ alkynyl, C₃₋₁₂ carbocycle and 3- to 12-membered heterocycle.

In some embodiments, for the compound or salt of Formula (I), (IA), (IB), (IC), (ID), and (IE), R⁶ and R⁷ are each hydrogen and R⁵ is C₁₋₄ alkyl optionally substituted with one or more substituents selected from halogen, —OR⁴¹, —C(O)R⁴¹, —C(O)OR⁴¹ and —CN. In some embodiments, R⁶ and R⁷ are each hydrogen and R⁵ is C₁₋₄ alkyl optionally substituted with one or more substituents selected from halogen, —OR⁴¹ wherein R⁴¹ is selected from hydrogen and C₁₋₅ alkyl optionally substituted with one or more substituents selected from halogen, —OH, —CN, and —NO₂. In some embodiments, R⁶ and R⁷ are each hydrogen and R⁵ is C₁₋₄ alkyl optionally substituted with one or more substituents selected from —OR⁴¹ wherein R⁴¹ is selected from hydrogen and C₁₋₅ alkyl optionally substituted with one or more substituents selected from halogen and —OH. In some embodiments, R⁶ and R⁷ are each hydrogen and R⁵ is selected from

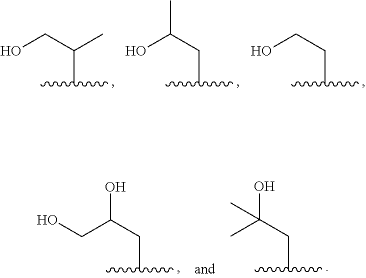

In some embodiments, for the compound or salt of Formula (I), (IA), (IB), (IC), (ID), and (IE), R⁶ and R⁷ are each hydrogen and R⁵ is C₁₋₄ alkyl optionally substituted with one or more substituents selected —OR⁴¹ and C₃₋₆ carbocycle wherein the C₃₋₆ carbocycle is optionally substituted with one or more substituents independently selected from halogen, —OR⁴¹, —NO₂, —CN, C₁₋₆ alkyl, and C₁₋₆ haloalkyl. In some embodiments, R⁶ and R⁷ are each hydrogen and R⁵ is C₁₋₄ alkyl is substituted with one or more substituents selected —OR⁴¹ wherein R⁴¹ is selected from hydrogen and C₁₋₅ alkyl; and C₃₋₆ carbocycle wherein C₃₋₆ carbocycle is optionally substituted with one or more substituents independently selected from halogen, —OR⁴¹, —NO₂, —CN, C₁₋₃ alkyl, C₁₋₃ haloalkyl. In some embodiments, R⁶ and R⁷ are each hydrogen and R⁵ is

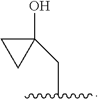 [SMILES: *CC1(O)CC1]

In some embodiments, for the compound or salt of Formula (I), (IA), (IB), (IC), (ID), and (IE), two of R⁵, R⁶ and R⁷ are hydrogen and the other of R⁵, R⁶ and R⁷ is selected from optionally substituted C₁₋₄ alkyl, optionally substituted C₃₋₁₀ carbocycle and optionally substituted 3- to 10-membered heterocycle.

In some embodiments, for the compound or salt of Formula (I), (IA), (IB), (IC), (ID), and (IE), two of R⁵, R⁶ and R⁷ are hydrogen and the other of R⁵, R⁶ and R⁷ is selected from methyl, ethyl, propyl, butyl, cyclopropyl, phenyl, and 9 or 10-membered heterocycle, wherein methyl, ethyl, propyl, butyl, cyclopropyl, phenyl, and 9 or 10-membered heterocycle each of which are optionally substituted by one or more substituents independently selected from: halogen, —OR⁴¹, —N(R⁴¹)₂, —C(O)R⁴¹, —C(O)N(R⁴¹)₂, —N(R⁴¹)C(O)R⁴¹, —N(R⁴¹)C(O)OR⁴¹, —C(O)OR⁴¹, ═O, and —CN; and

C₁₋₅ alkyl optionally substituted with one or more substituents independently selected from halogen, —OR⁴¹, —N(R⁴¹)₂, —C(O)R⁴¹, —C(O)N(R⁴¹)₂, —N(R₄₁)C(O)R⁴¹, —N(R⁴¹)C(O)OR⁴¹, —C(O)OR⁴¹, ═O, and —CN, C₃₋₁₂ carbocycle and 3- to 12-membered heterocycle;

C₃₋₆ carbocycle and 3- to 6-membered heterocycle each of which is optionally substituted with one or more substituents independently selected from halogen, —OR⁴¹, —N(R⁴¹)₂, —C(O)R⁴¹, —C(O)OR⁴¹, —NO₂, —CN, C₁₋₆ alkyl, C₁₋₆ haloalkyl, C₂₋₆ alkenyl, C₂₋₆ alkynyl, C₃₋₁₂ carbocycle and 3- to 12-membered heterocycle.

In some embodiments, for the compound or salt of Formula (I), (IA), (IB), (IC), (ID), and (IE), two of R⁵, R⁶ and R⁷ are hydrogen and the other of R⁵, R⁶ and R⁷ is selected from is selected from methyl, ethyl, propyl, butyl each of which is optionally substituted by one or more substituents independently selected from: halogen, —OR⁴¹, —N(R⁴¹)₂, —C(O)R⁴¹, —C(O)N(R⁴¹)₂, ——N(R⁴¹)C(O)R⁴¹, —N(R⁴¹)C(O)OR⁴¹, —C(O)OR⁴¹, ═O, and —CN; and

C₃₋₆ carbocycle and 3- to 6-membered heterocycle each of which is optionally substituted with one or more substituents independently selected from halogen, —OR⁴¹, —N(R⁴¹)₂, —C(O)R⁴¹, —C(O)OR⁴¹, —NO₂, —CN, C₁₋₆ alkyl, C₁₋₆ haloalkyl, C₂₋₆ alkenyl, C₂₋₆ alkynyl, C₃₋₁₂ carbocycle and 3- to 12-membered heterocycle. In some embodiments, two of R⁵, R⁶ and R⁷ are hydrogen and the other of R⁵, R⁶ and R⁷ is selected from:

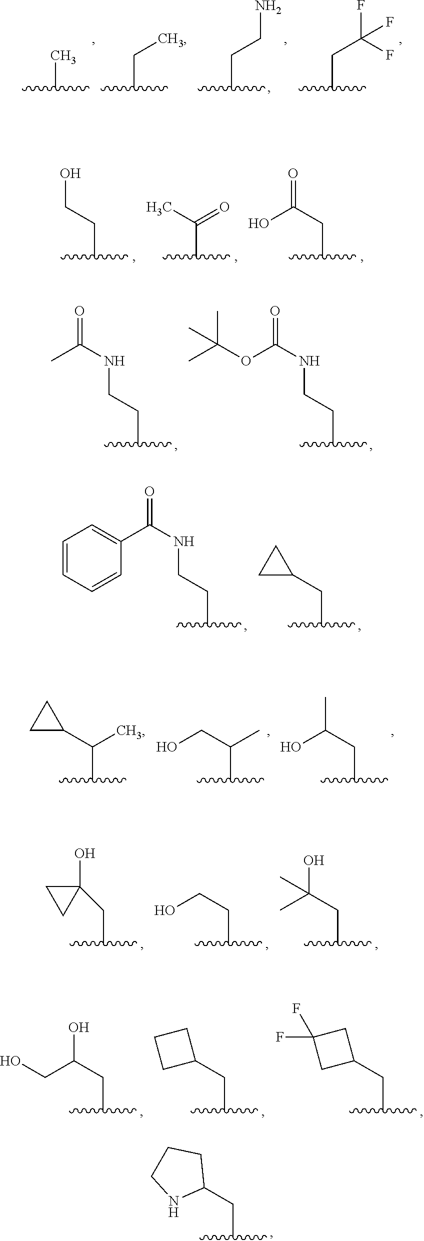

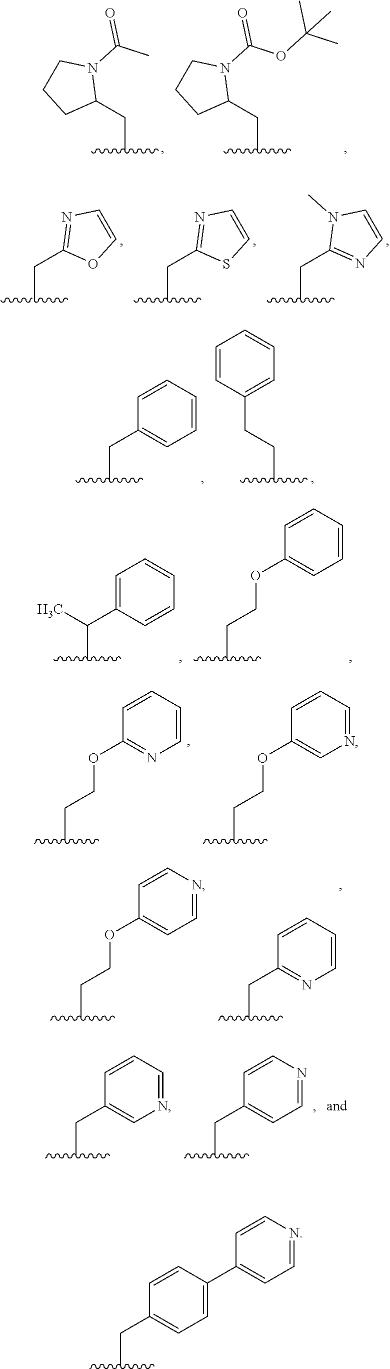

In some embodiments, for the compound or salt of Formula (I), (IA), (IB), (IC), (ID), and (IE), two of R⁵, R⁶ and R⁷ are hydrogen and the other of R⁵, R⁶ and R⁷ is cyclopropyl optionally substituted by one or more substituents independently selected from: halogen, —OR⁴¹, —N(R⁴¹)₂, —C(O)R⁴¹, —C(O)N(R⁴¹)₂, —N(R⁴¹)C(O)R⁴¹, —N(R⁴¹)C(O)OR⁴¹, —C(O)OR⁴¹, ═O, and —CN; and optionally substituted C₃₋₆ carbocycle and optionally substituted 3- to 6-membered heterocycle. In some embodiments, two of R⁵, R⁶ and R⁷ are hydrogen and the other of R⁵, R⁶ and R⁷ is selected from:

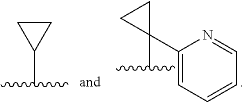

In some embodiments, for the compound or salt of Formula (I), (IA), (IB), (IC), (ID), and (IE), two of R⁵, R⁶ and R⁷ are hydrogen and the other of R⁵, R⁶ and R⁷ is phenyl is optionally substituted by one or more substituents independently selected from: halogen, —OR⁴¹, —N(R⁴¹)₂, —C(O)R⁴¹, —C(O)N(R⁴¹)₂, —N(R⁴¹)C(O)R⁴¹, —N(R⁴¹)C(O)OR⁴¹, —C(O)OR⁴¹, ═O, and —CN; and optionally substituted C₃₋₆ carbocycle and optionally substituted 3- to 6-membered heterocycle. In some embodiments, two of R⁵, R⁶ and R⁷ are hydrogen and the other of R⁵, R⁶ and R⁷ is selected from:

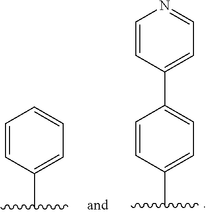 [SMILES: *c1ccccc1 ; *c1ccc(-c2ccncc2)cc1]

In some embodiments, for the compound or salt of Formula (I), (IA), (IB), (IC), (ID), and (IE), two of R⁵, R⁶ and R⁷ are hydrogen and the other of R⁵, R⁶ and R⁷ is 9 or 10-membered heterocycle optionally substituted by one or more substituents independently selected from: halogen, —OR⁴¹, —N(R⁴¹)₂, —C(O)R⁴¹, —C(O)N(R⁴¹)₂, —N(R⁴¹)C(O)R⁴¹, —N(R⁴¹)C(O)OR⁴¹, —C(O)OR⁴¹, ═O, and —CN. In some embodiments, two of R⁵, R⁶ and R⁷ are hydrogen and the other of R⁵, R⁶ and R⁷ is

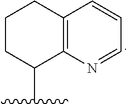 [SMILES: *C1CCCc2cccnc21]

In some embodiments, for the compound or salt of Formula (I), (IA), (IB), (IC), (ID), and (IE), two of R⁵, R⁶ and R⁷ are hydrogen and the other of R⁵, R⁶ and R⁷ is selected from:

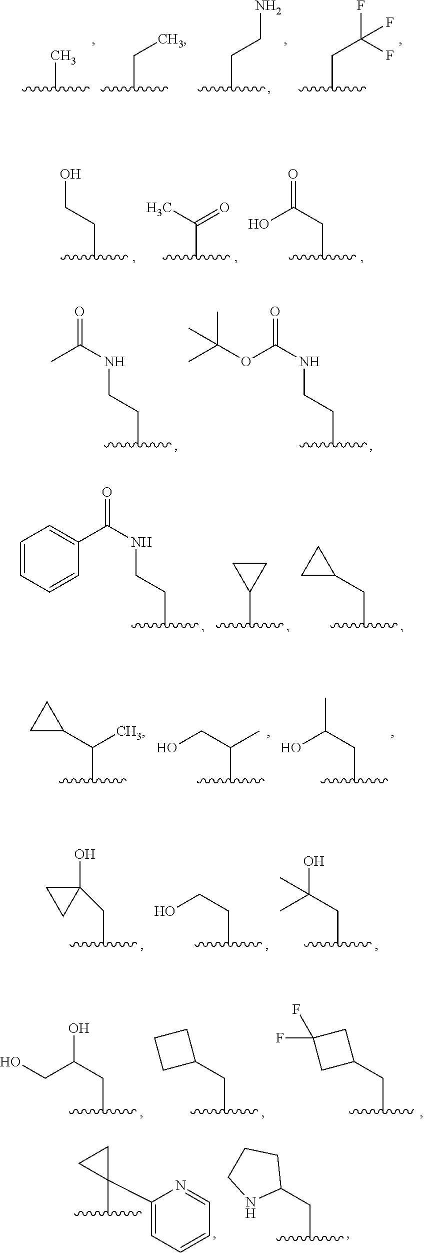

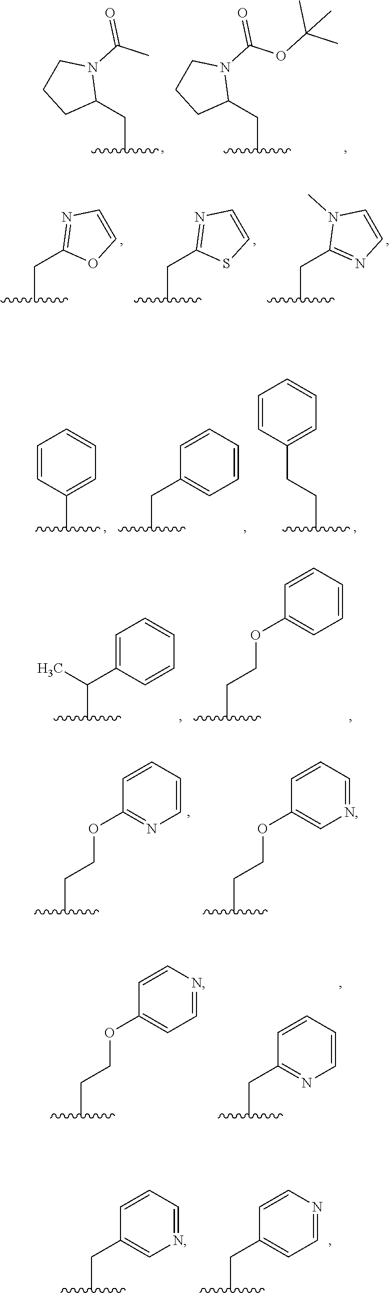

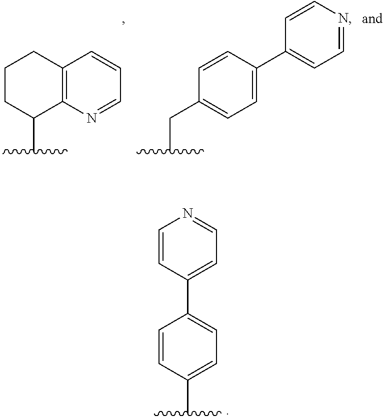 [SMILES: *C1CCCc2cccnc21 ; *Cc1ccc(-c2ccncc2)cc1 ; "and" ; *c1ccc(-c2ccncc2)cc1]

In some embodiments, for the compound or salt of Formula (I), (IA), (IB), (IC), (ID), and (IE), one of R⁵, R⁶ and R⁷ is hydrogen and the other two of R⁵, R⁶ and R⁷ are independently selected from optionally substituted C₁₋₆ alkyl, optionally substituted C₃₋₁₂ carbocycle and optionally substituted 3- to 12-membered heterocycle. In some embodiments, one of R⁵, R⁶ and R⁷ is hydrogen and the other two of R⁵, R⁶ and R⁷ are independently selected from C₁₋₄ alkyl and C₁₋₄ haloalkyl. In some embodiments, R⁵, R⁶ and R⁷ is hydrogen and the other two of R⁵, R⁶ and R⁷ are independently selected from methyl and ethyl.

In some embodiments, for the compound or salt of Formula (I), (IA), (IB), (IC), (ID), and (IE), R⁵ is hydrogen. In some embodiments, R⁵ is hydrogen and both R⁶ and R⁷are independently selected from optionally substituted C₁₋₆ alkyl, optionally substituted C₃₋₁₂ carbocycle and optionally substituted 3- to 12-membered heterocycle. In some embodiments, R⁵ is hydrogen; R⁶ is independently selected from optionally substituted C₁₋₆ alkyl, optionally substituted C₃₋₁₂ carbocycle and optionally substituted 3- to 12-membered heterocycle; and R⁷ is independently selected from optionally substituted C₃₋₁₂ carbocycle and optionally substituted 3- to 12-membered heterocycle. In some embodiments, R⁵ is hydrogen and both R⁶ and R⁷ are independently selected from C₁₋₄ alkyl and C₁₋₄ haloalkyl. In some embodiments, R⁵ is hydrogen and both R⁶ and R⁷ are independently selected from methyl and ethyl. In some embodiments, R⁵ is hydrogen and both R⁶ and R⁷ are represented by

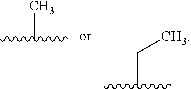

In some embodiments, for the compound or salt of Formula (I), (IA), (IB), (IC), (ID), and (IE), R⁶ is hydrogen. In some embodiments, R⁶ is hydrogen and both R⁵ and R⁷ are independently selected from optionally substituted C₁₋₆ alkyl, optionally substituted C₃₋₁₂ carbocycle and optionally substituted 3- to 12-membered heterocycle In some embodiments, R⁶ is hydrogen and both R⁵ and R⁷ are independently selected from C₁₋₄ alkyl and C₁₋₄ haloalkyl. In some embodiments, R⁶ is hydrogen and both R⁵ and R⁷ are independently selected from methyl and ethyl. In some embodiments, R⁶ is hydrogen and both R⁵ and R⁷ are represented by

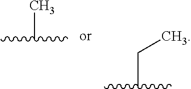

In some embodiments, for the compound or salt of Formula (I), (IA), (IB), (IC), (ID), and (IE), R⁶ and R⁷ come together to form an optionally substituted 5- to 6-membered heterocycle and R⁵ is hydrogen.

In some embodiments, for the compound or salt of Formula (I), (IA), (IB), (IC), (ID), and (IE), R⁶ and R⁷ come together to form an optionally substituted 5- to 6-membered heterocycle and R⁵ is hydrogen. In some embodiments, R⁶ and R⁷ come together to form an optionally substituted 5- to 6-membered saturated heterocycle, and R⁵ is hydrogen. In some embodiments, R⁶ and R⁷ come together to form optionally substituted pyrrolidine, optionally substituted piperidine, optionally substituted piperazine or optionally substituted morpholine and R⁵ is hydrogen. In some embodiments, R⁶ and R⁷ come together to form an optionally substituted 5- to 6-membered saturated heterocycle represented by:

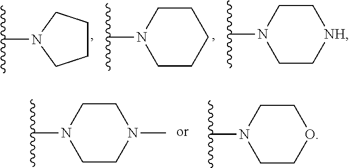

In some embodiments, for the compound or salt of Formula (I), (IA), (IB), (IC), (ID), and (IE), R⁵ and R⁶ come together to form an optionally substituted 5- to 8-membered heterocycle, and R⁷ is selected from hydrogen, optionally substituted C₁₋₆ alkyl, optionally substituted C₃₋₁₂ carbocycle and optionally substituted 3- to 12-membered heterocycle. In some embodiments, when R⁵ and R⁶ come together to form an optionally substituted 5- to 8-membered heterocycle the optionally substituted 5- to 8-membered heterocycle is a cyclic sulfonimidamide.

In some embodiments, for the compound or salt of Formula (I), (IA), (IB), (IC), (ID), and (IE), R⁵ and R⁶ come together to form an optionally substituted 5-membered heterocycle and R⁷ is selected from hydrogen, and C₁₋₃ alkyl. In some embodiments, the optionally substituted 5-membered heterocycle of R⁵ and R⁶ is represented by

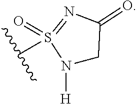 [SMILES: *S1(=O)=NC(=O)CN1]

In some embodiments, the compound or salt of Formula (I), (IA), (IB), (IC), (ID), and (IE) is selected from:

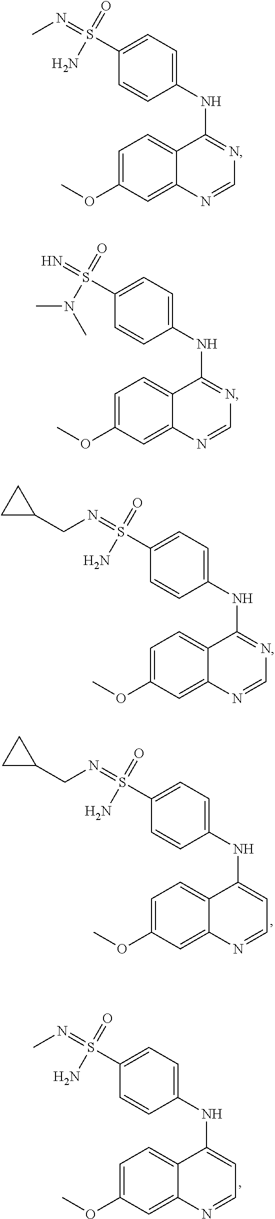 [SMILES: CN=S(N)(=O)c1ccc(Nc2ncnc3cc(OC)ccc23)cc1 ; COc1ccc2c(Nc3ccc(S(=N)(=O)N(C)C)cc3)ncnc2c1 ; COc1ccc2c(Nc3ccc(S(N)(=O)=NCC4CC4)cc3)ncnc2c1 ; COc1ccc2c(Nc3ccc(S(N)(=O)=NCC4CC4)cc3)ccnc2c1 ; CN=S(N)(=O)c1ccc(Nc2ccnc3cc(OC)ccc23)cc1]

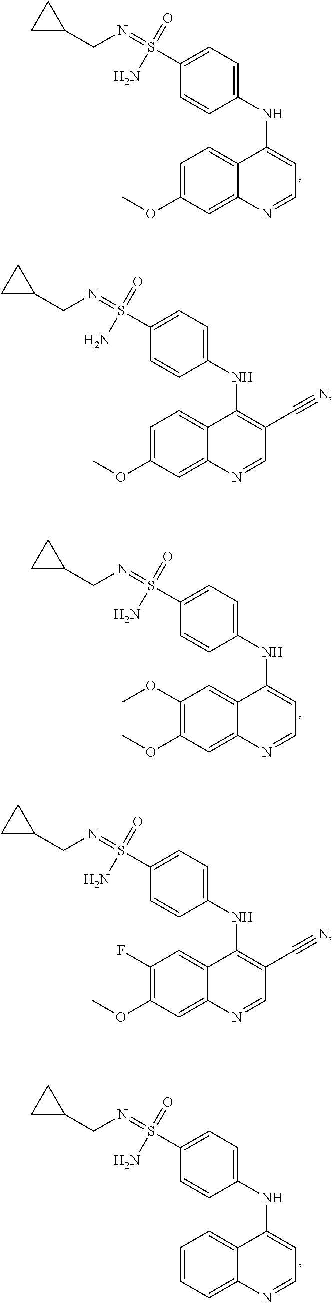 [SMILES: COc1ccc2c(Nc3ccc(S(N)(=O)=NCC4CC4)cc3)ccnc2c1 ; COc1ccc2c(Nc3ccc(S(N)(=O)=NCC4CC4)cc3)c(C#N)cnc2c1 ; COc1cc2nccc(Nc3ccc(S(N)(=O)=NCC4CC4)cc3)c2cc1OC ; COc1cc2ncc(C#N)c(Nc3ccc(S(N)(=O)=NCC4CC4)cc3)c2cc1F ; NS(=O)(=NCC1CC1)c1ccc(Nc2ccnc3ccccc23)cc1]

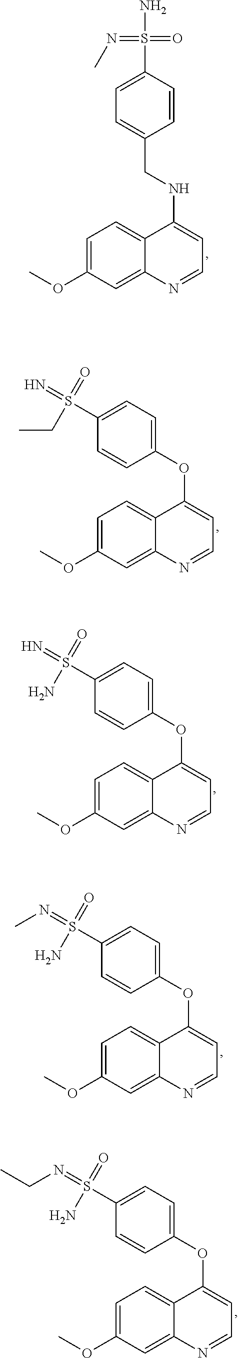 [SMILES: CN=S(N)(=O)c1ccc(CNc2ccnc3cc(OC)ccc23)cc1 ; CCS(=N)(=O)c1ccc(Oc2ccnc3cc(OC)ccc23)cc1 ; COc1ccc2c(Oc3ccc(S(=N)(N)=O)cc3)ccnc2c1 ; CN=S(N)(=O)c1ccc(Oc2ccnc3cc(OC)ccc23)cc1 ; CCN=S(N)(=O)c1ccc(Oc2ccnc3cc(OC)ccc23)cc1]

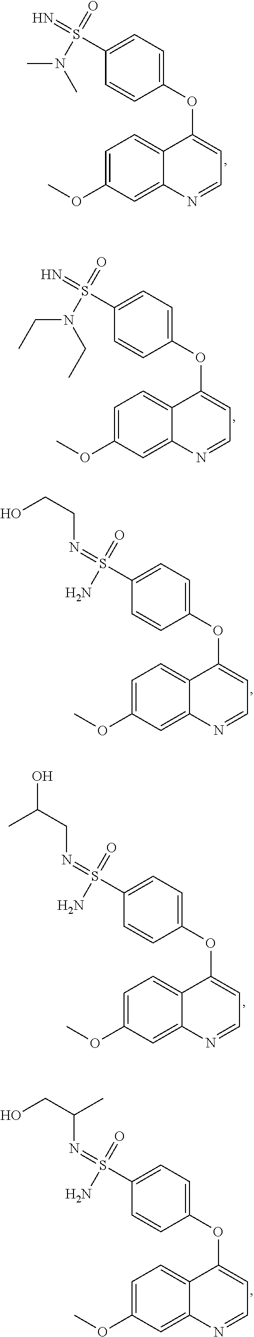 [SMILES: COc1ccc2c(Oc3ccc(S(=N)(=O)N(C)C)cc3)ccnc2c1 ; CCN(CC)S(=N)(=O)c1ccc(Oc2ccnc3cc(OC)ccc23)cc1 ; COc1ccc2c(Oc3ccc(S(N)(=O)=NCCO)cc3)ccnc2c1 ; COc1ccc2c(Oc3ccc(S(N)(=O)=NCC(C)O)cc3)ccnc2c1 ; COc1ccc2c(Oc3ccc(S(N)(=O)=NC(C)CO)cc3)ccnc2c1]

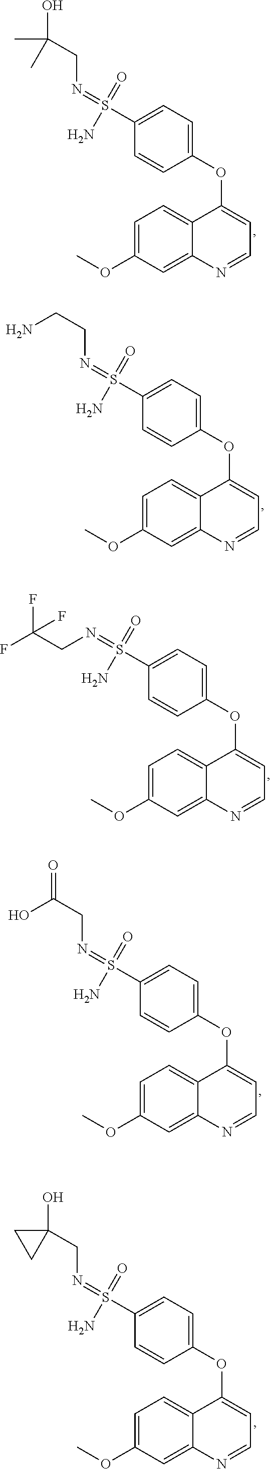 [SMILES: COc1ccc2c(Oc3ccc(S(N)(=O)=NCC(C)(C)O)cc3)ccnc2c1 ; COc1ccc2c(Oc3ccc(S(N)(=O)=NCCN)cc3)ccnc2c1 ; COc1ccc2c(Oc3ccc(S(N)(=O)=NCC(F)(F)F)cc3)ccnc2c1 ; COc1ccc2c(Oc3ccc(S(N)(=O)=NCC(=O)O)cc3)ccnc2c1 ; COc1ccc2c(Oc3ccc(S(N)(=O)=NCC4(O)CC4)cc3)ccnc2c1]

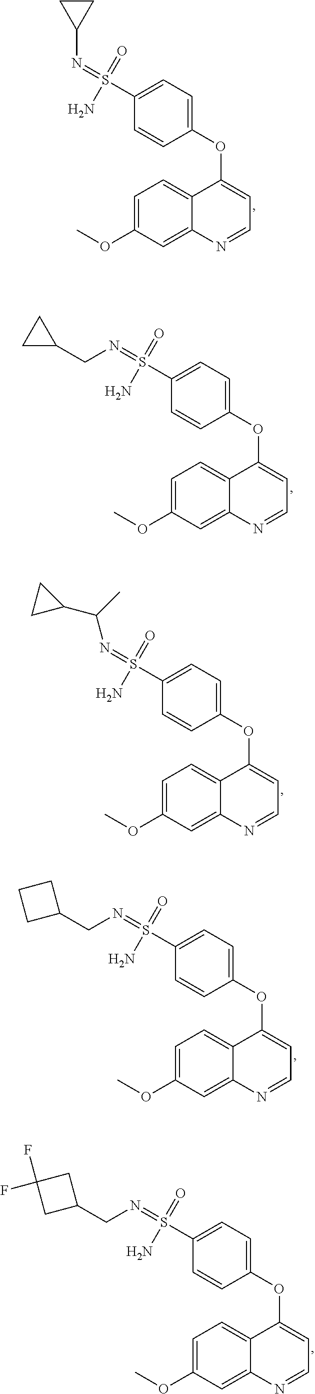 [SMILES: COc1ccc2c(Oc3ccc(S(N)(=O)=NC4CC4)cc3)ccnc2c1 ; COc1ccc2c(Oc3ccc(S(N)(=O)=NCC4CC4)cc3)ccnc2c1 ; COc1ccc2c(Oc3ccc(S(N)(=O)=NC(C)C4CC4)cc3)ccnc2c1 ; COc1ccc2c(Oc3ccc(S(N)(=O)=NCC4CCC4)cc3)ccnc2c1 ; COc1ccc2c(Oc3ccc(S(N)(=O)=NCC4CC(F)(F)C4)cc3)ccnc2c1]

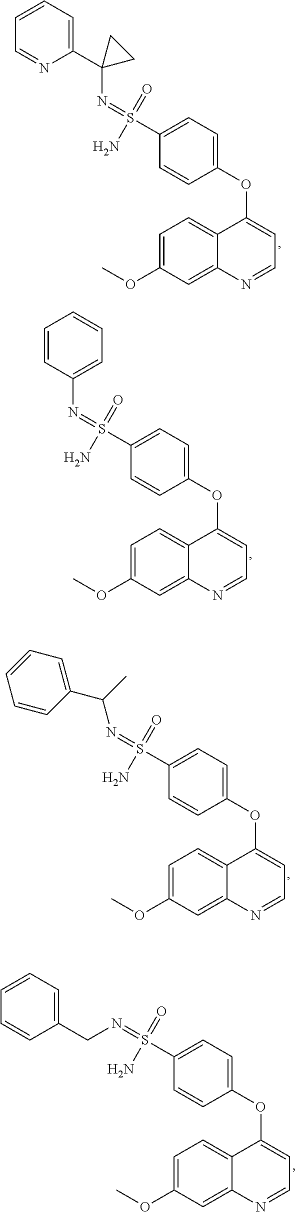 [SMILES: COc1ccc2c(Oc3ccc(S(N)(=O)=NC4(c5ccccn5)CC4)cc3)ccnc2c1 ; COc1ccc2c(Oc3ccc(S(N)(=O)=Nc4ccccc4)cc3)ccnc2c1 ; COc1ccc2c(Oc3ccc(S(N)(=O)=NC(C)c4ccccc4)cc3)ccnc2c1 ; COc1ccc2c(Oc3ccc(S(N)(=O)=NCc4ccccc4)cc3)ccnc2c1]

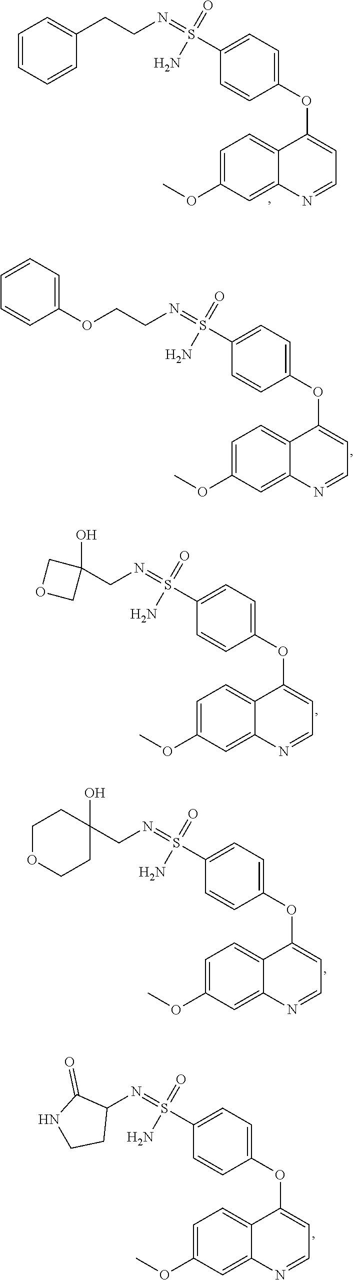 [SMILES: COc1ccc2c(Oc3ccc(S(N)(=O)=NCCc4ccccc4)cc3)ccnc2c1 ; COc1ccc2c(Oc3ccc(S(N)(=O)=NCCOc4ccccc4)cc3)ccnc2c1 ; COc1ccc2c(Oc3ccc(S(N)(=O)=NCC4(O)COC4)cc3)ccnc2c1 ; COc1ccc2c(Oc3ccc(S(N)(=O)=NCC4(O)CCOCC4)cc3)ccnc2c1 ; COc1ccc2c(Oc3ccc(S(N)(=O)=NC4CCNC4=O)cc3)ccnc2c1]

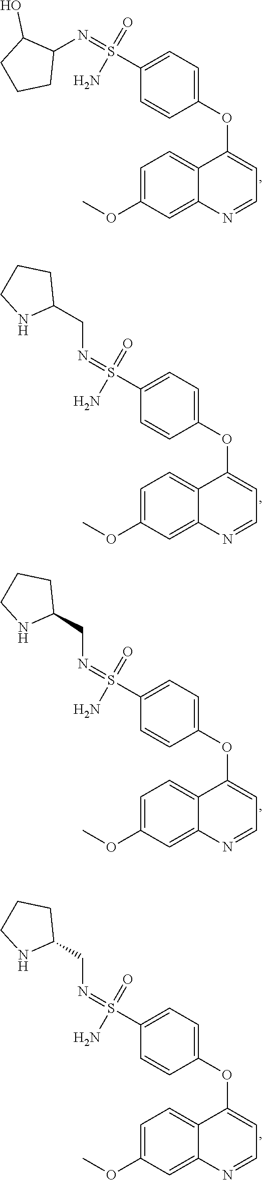 [SMILES: COc1ccc2c(Oc3ccc(S(N)(=O)=NC4CCCC4O)cc3)ccnc2c1 ; COc1ccc2c(Oc3ccc(S(N)(=O)=NCC4CCCN4)cc3)ccnc2c1 ; COc1ccc2c(Oc3ccc(S(N)(=O)=NC[C@@H]4CCCN4)cc3)ccnc2c1 ; COc1ccc2c(Oc3ccc(S(N)(=O)=NC[C@H]4CCCN4)cc3)ccnc2c1]

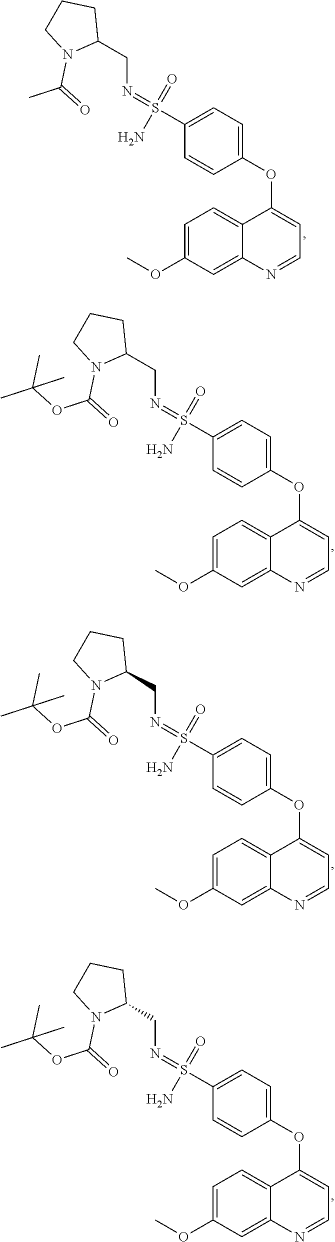 [SMILES: COc1ccc2c(Oc3ccc(S(N)(=O)=NCC4CCCN4C(C)=O)cc3)ccnc2c1 ; COc1ccc2c(Oc3ccc(S(N)(=O)=NCC4CCCN4C(=O)OC(C)(C)C)cc3)ccnc2c1 ; COc1ccc2c(Oc3ccc(S(N)(=O)=NC[C@@H]4CCCN4C(=O)OC(C)(C)C)cc3)ccnc2c1 ; COc1ccc2c(Oc3ccc(S(N)(=O)=NC[C@H]4CCCN4C(=O)OC(C)(C)C)cc3)ccnc2c1]

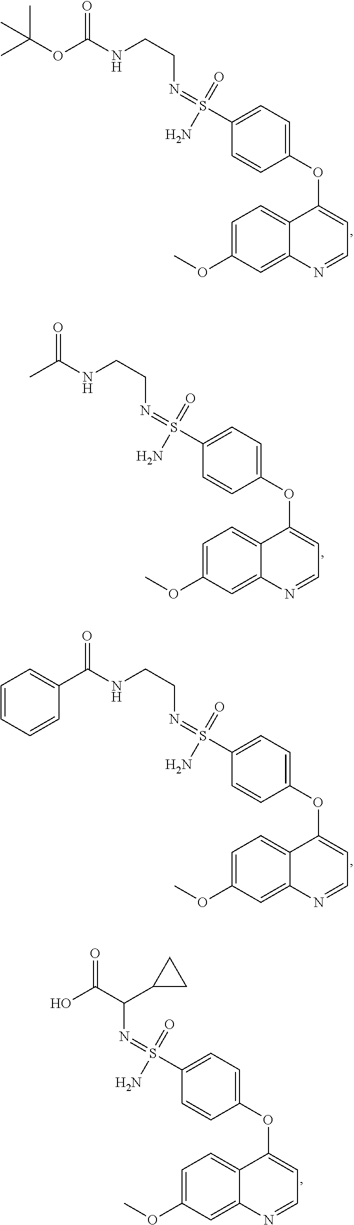 [SMILES: COc1ccc2c(Oc3ccc(S(N)(=O)=NCCNC(=O)OC(C)(C)C)cc3)ccnc2c1 ; COc1ccc2c(Oc3ccc(S(N)(=O)=NCCNC(C)=O)cc3)ccnc2c1 ; COc1ccc2c(Oc3ccc(S(N)(=O)=NCCNC(=O)c4ccccc4)cc3)ccnc2c1 ; COc1ccc2c(Oc3ccc(S(N)(=O)=NC(C(=O)O)C4CC4)cc3)ccnc2c1]

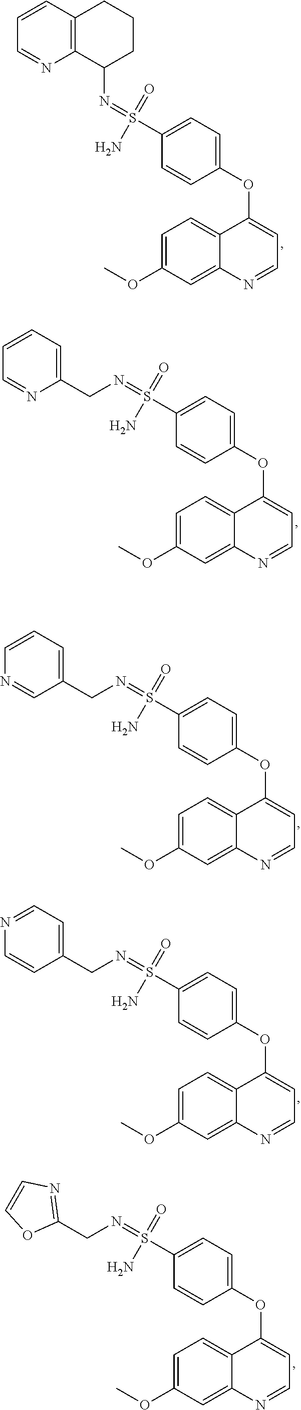 [SMILES: COc1ccc2c(Oc3ccc(S(N)(=O)=NC4CCCc5cccnc54)cc3)ccnc2c1 ; COc1ccc2c(Oc3ccc(S(N)(=O)=NCc4ccccn4)cc3)ccnc2c1 ; COc1ccc2c(Oc3ccc(S(N)(=O)=NCc4cccnc4)cc3)ccnc2c1 ; COc1ccc2c(Oc3ccc(S(N)(=O)=NCc4ccncc4)cc3)ccnc2c1 ; COc1ccc2c(Oc3ccc(S(N)(=O)=NCc4ncco4)cc3)ccnc2c1]

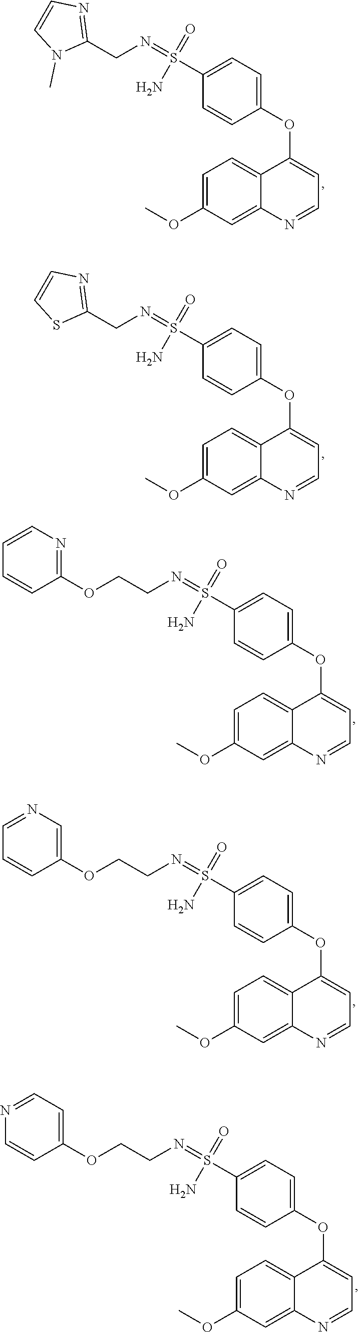 [SMILES: COc1ccc2c(Oc3ccc(S(N)(=O)=NCc4nccn4C)cc3)ccnc2c1 ; COc1ccc2c(Oc3ccc(S(N)(=O)=NCc4nccs4)cc3)ccnc2c1 ; COc1ccc2c(Oc3ccc(S(N)(=O)=NCCOc4ccccn4)cc3)ccnc2c1 ; COc1ccc2c(Oc3ccc(S(N)(=O)=NCCOc4cccnc4)cc3)ccnc2c1 ; COc1ccc2c(Oc3ccc(S(N)(=O)=NCCOc4ccncc4)cc3)ccnc2c1]

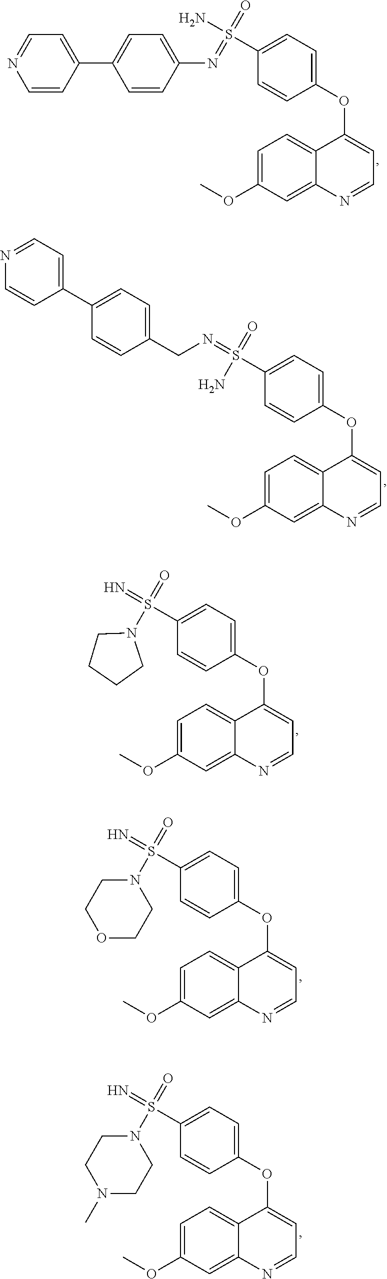 [SMILES: COc1ccc2c(Oc3ccc(S(N)(=O)=Nc4ccc(-c5ccncc5)cc4)cc3)ccnc2c1 ; COc1ccc2c(Oc3ccc(S(N)(=O)=NCc4ccc(-c5ccncc5)cc4)cc3)ccnc2c1 ; COc1ccc2c(Oc3ccc(S(=N)(=O)N4CCCC4)cc3)ccnc2c1 ; COc1ccc2c(Oc3ccc(S(=N)(=O)N4CCOCC4)cc3)ccnc2c1 ; COc1ccc2c(Oc3ccc(S(=N)(=O)N4CCN(C)CC4)cc3)ccnc2c1]

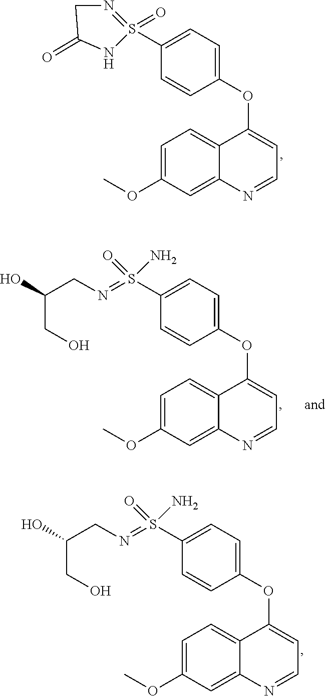 [SMILES: COc1ccc2c(Oc3ccc(S4(=O)=NCC(=O)N4)cc3)ccnc2c1 ; COc1ccc2c(Oc3ccc(S(N)(=O)=NC[C@H](O)CO)cc3)ccnc2c1 ; COc1ccc2c(Oc3ccc(S(N)(=O)=NC[C@@H](O)CO)cc3)ccnc2c1]

or a tautomer thereof.

In some embodiments, the tautomer of the compound or salt of Formula (I), (IA), (IB), (IC), (ID), and (IE) is selected from:

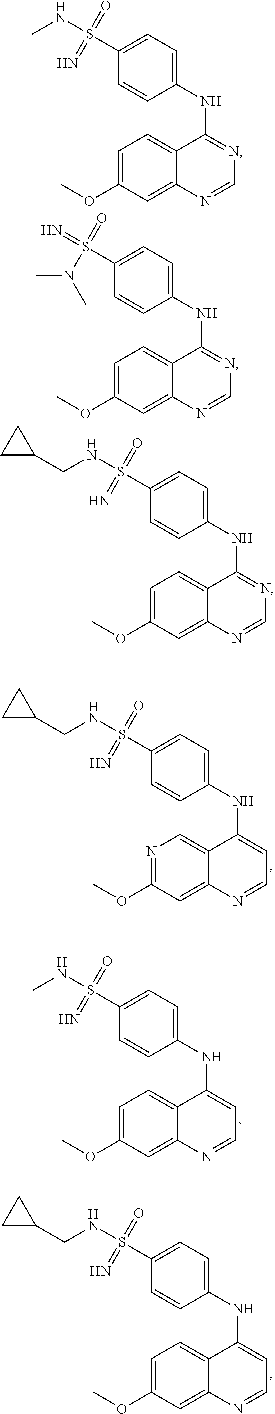

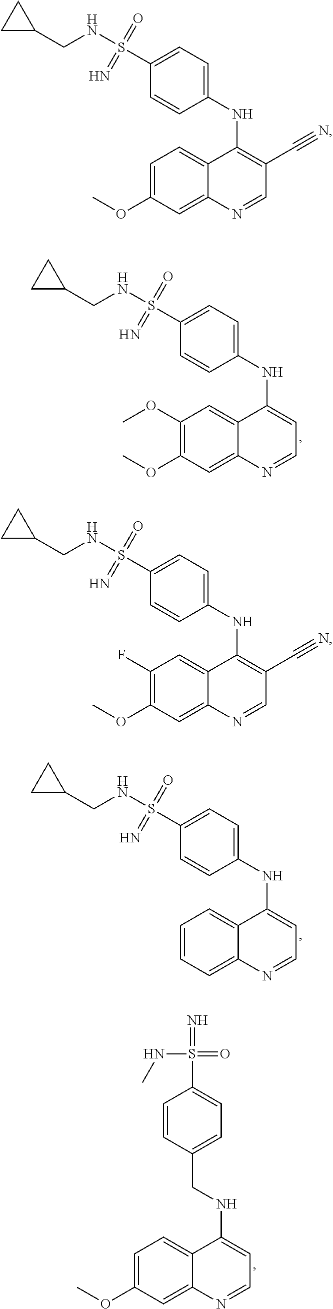 [SMILES: COc1ccc2c(Nc3ccc(S(=N)(=O)NCC4CC4)cc3)c(C#N)cnc2c1 ; COc1cc2nccc(Nc3ccc(S(=N)(=O)NCC4CC4)cc3)c2cc1OC ; COc1cc2ncc(C#N)c(Nc3ccc(S(=N)(=O)NCC4CC4)cc3)c2cc1F ; N=S(=O)(NCC1CC1)c1ccc(Nc2ccnc3ccccc23)cc1 ; CNS(=N)(=O)c1ccc(CNc2ccnc3cc(OC)ccc23)cc1]

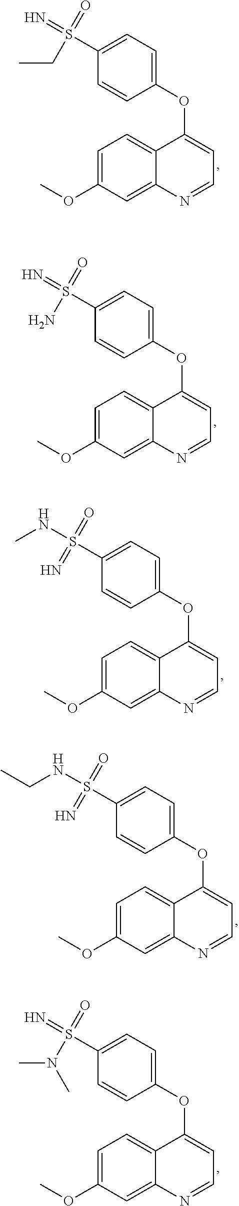 [SMILES: CCS(=N)(=O)c1ccc(Oc2ccnc3cc(OC)ccc23)cc1 ; COc1ccc2c(Oc3ccc(S(=N)(N)=O)cc3)ccnc2c1 ; CNS(=N)(=O)c1ccc(Oc2ccnc3cc(OC)ccc23)cc1 ; CCNS(=N)(=O)c1ccc(Oc2ccnc3cc(OC)ccc23)cc1 ; COc1ccc2c(Oc3ccc(S(=N)(=O)N(C)C)cc3)ccnc2c1]

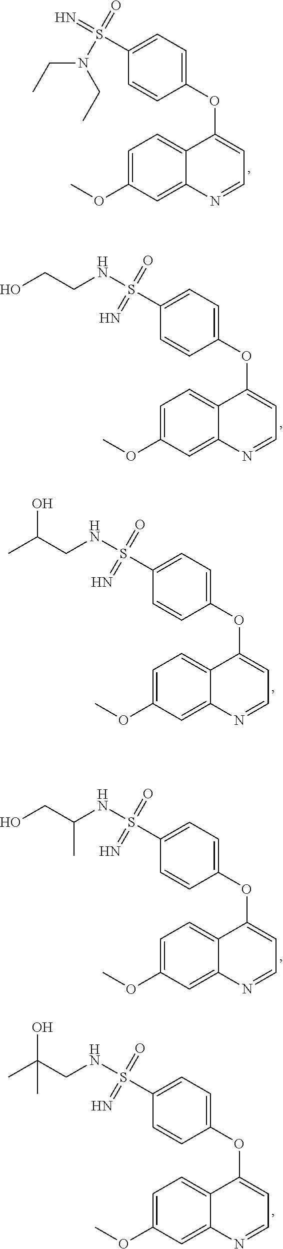 [SMILES: CCN(CC)S(=N)(=O)c1ccc(Oc2ccnc3cc(OC)ccc23)cc1 ; COc1ccc2c(Oc3ccc(S(=N)(=O)NCCO)cc3)ccnc2c1 ; COc1ccc2c(Oc3ccc(S(=N)(=O)NCC(C)O)cc3)ccnc2c1 ; COc1ccc2c(Oc3ccc(S(=N)(=O)NC(C)CO)cc3)ccnc2c1 ; COc1ccc2c(Oc3ccc(S(=N)(=O)NCC(C)(C)O)cc3)ccnc2c1]

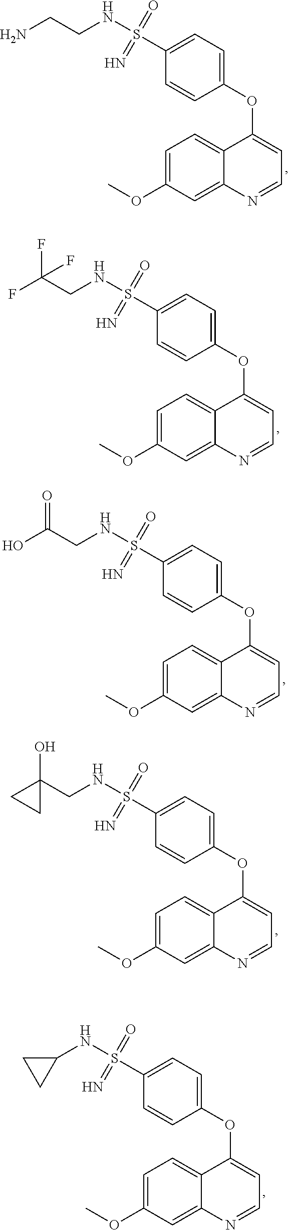 [SMILES: COc1ccc2c(Oc3ccc(S(=N)(=O)NCCN)cc3)ccnc2c1 ; COc1ccc2c(Oc3ccc(S(=N)(=O)NCC(F)(F)F)cc3)ccnc2c1 ; COc1ccc2c(Oc3ccc(S(=N)(=O)NCC(=O)O)cc3)ccnc2c1 ; COc1ccc2c(Oc3ccc(S(=N)(=O)NCC4(O)CC4)cc3)ccnc2c1 ; COc1ccc2c(Oc3ccc(S(=N)(=O)NC4CC4)cc3)ccnc2c1]

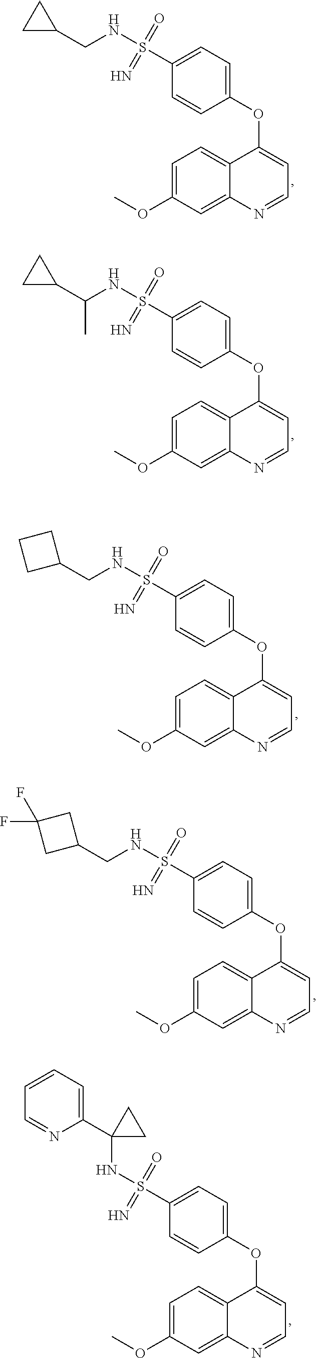 [SMILES: COc1ccc2c(Oc3ccc(S(=N)(=O)NCC4CC4)cc3)ccnc2c1 ; COc1ccc2c(Oc3ccc(S(=N)(=O)NC(C)C4CC4)cc3)ccnc2c1 ; COc1ccc2c(Oc3ccc(S(=N)(=O)NCC4CCC4)cc3)ccnc2c1 ; COc1ccc2c(Oc3ccc(S(=N)(=O)NCC4CC(F)(F)C4)cc3)ccnc2c1 ; COc1ccc2c(Oc3ccc(S(=N)(=O)NC4(c5ccccn5)CC4)cc3)ccnc2c1]

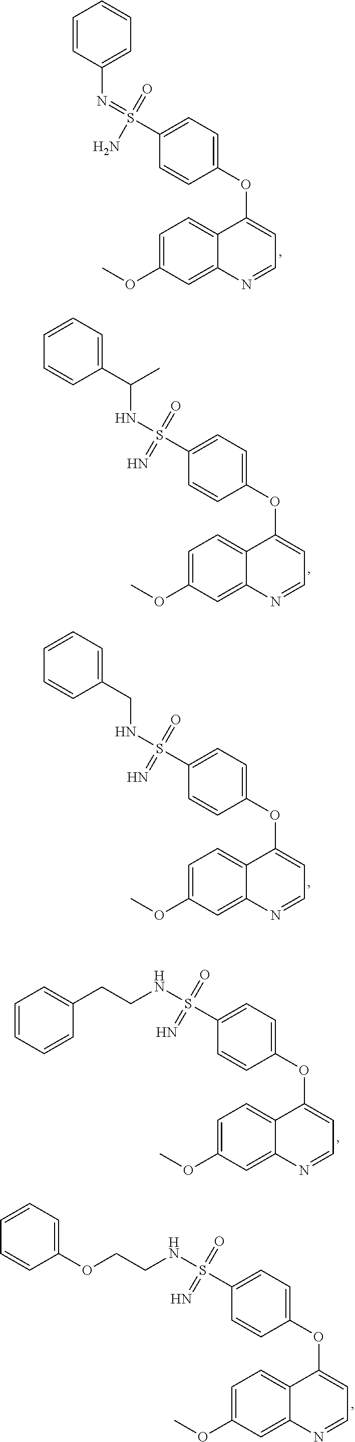 [SMILES: COc1ccc2c(Oc3ccc(S(N)(=O)=Nc4ccccc4)cc3)ccnc2c1 ; COc1ccc2c(Oc3ccc(S(=N)(=O)NC(C)c4ccccc4)cc3)ccnc2c1 ; COc1ccc2c(Oc3ccc(S(=N)(=O)NCc4ccccc4)cc3)ccnc2c1 ; COc1ccc2c(Oc3ccc(S(=N)(=O)NCCc4ccccc4)cc3)ccnc2c1 ; COc1ccc2c(Oc3ccc(S(=N)(=O)NCCOc4ccccc4)cc3)ccnc2c1]

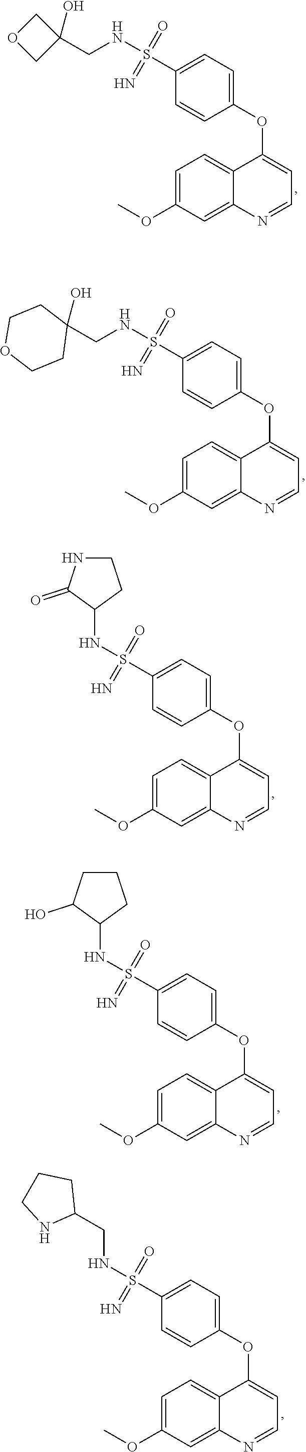 [SMILES: COc1ccc2c(Oc3ccc(S(=N)(=O)NCC4(O)COC4)cc3)ccnc2c1 ; COc1ccc2c(Oc3ccc(S(=N)(=O)NCC4(O)CCOCC4)cc3)ccnc2c1 ; COc1ccc2c(Oc3ccc(S(=N)(=O)NC4CCNC4=O)cc3)ccnc2c1 ; COc1ccc2c(Oc3ccc(S(=N)(=O)NC4CCCC4O)cc3)ccnc2c1 ; COc1ccc2c(Oc3ccc(S(=N)(=O)NCC4CCCN4)cc3)ccnc2c1]

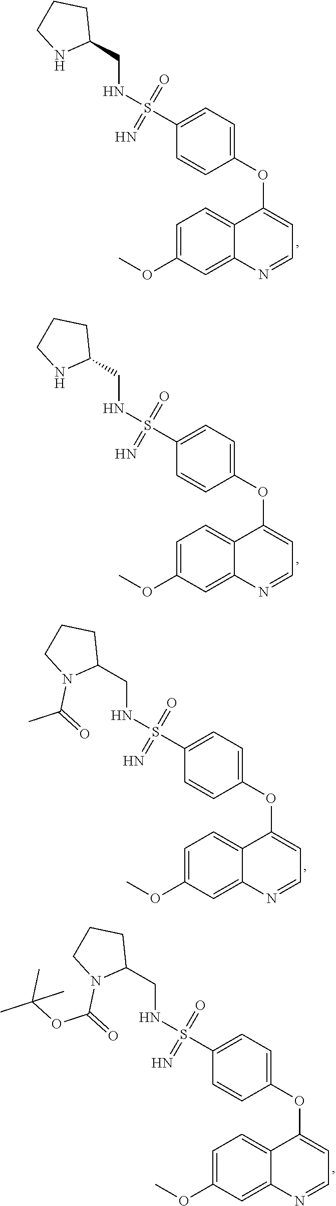 [SMILES: COc1ccc2c(Oc3ccc(S(=N)(=O)NC[C@@H]4CCCN4)cc3)ccnc2c1 ; COc1ccc2c(Oc3ccc(S(=N)(=O)NC[C@H]4CCCN4)cc3)ccnc2c1 ; COc1ccc2c(Oc3ccc(S(=N)(=O)NCC4CCCN4C(C)=O)cc3)ccnc2c1 ; COc1ccc2c(Oc3ccc(S(=N)(=O)NCC4CCCN4C(=O)OC(C)(C)C)cc3)ccnc2c1]

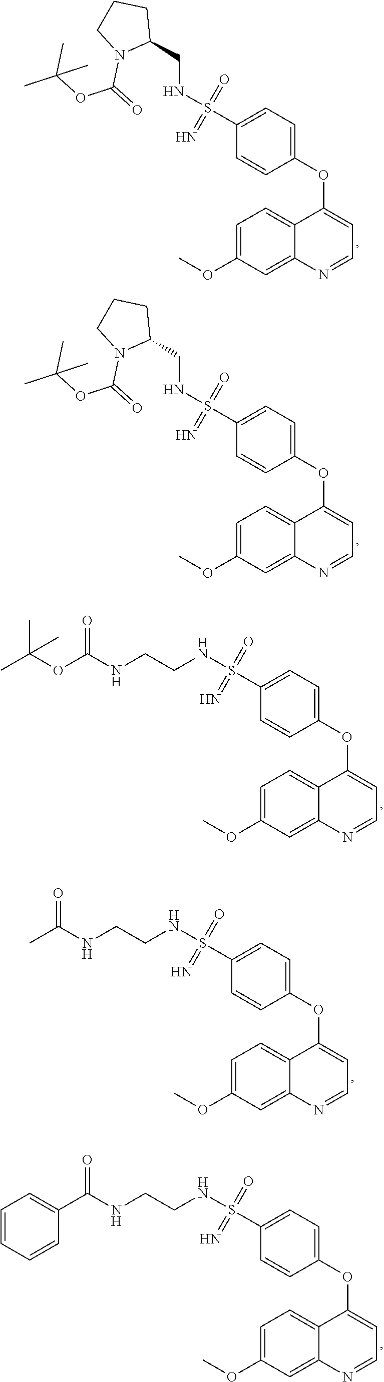 [SMILES: COc1ccc2c(Oc3ccc(S(=N)(=O)NC[C@@H]4CCCN4C(=O)OC(C)(C)C)cc3)ccnc2c1 ; COc1ccc2c(Oc3ccc(S(=N)(=O)NC[C@H]4CCCN4C(=O)OC(C)(C)C)cc3)ccnc2c1 ; COc1ccc2c(Oc3ccc(S(=N)(=O)NCCNC(=O)OC(C)(C)C)cc3)ccnc2c1 ; COc1ccc2c(Oc3ccc(S(=N)(=O)NCCNC(C)=O)cc3)ccnc2c1 ; COc1ccc2c(Oc3ccc(S(=N)(=O)NCCNC(=O)c4ccccc4)cc3)ccnc2c1]

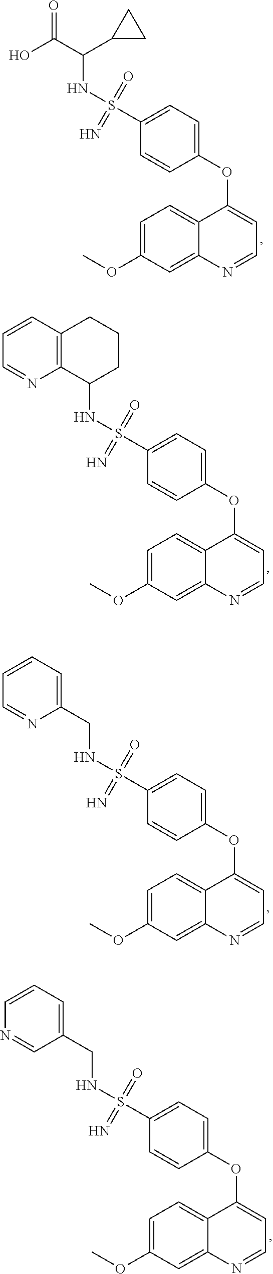 [SMILES: COc1ccc2c(Oc3ccc(S(=N)(=O)NC(C(=O)O)C4CC4)cc3)ccnc2c1 ; COc1ccc2c(Oc3ccc(S(=N)(=O)NC4CCCc5cccnc54)cc3)ccnc2c1 ; COc1ccc2c(Oc3ccc(S(=N)(=O)NCc4ccccn4)cc3)ccnc2c1 ; COc1ccc2c(Oc3ccc(S(=N)(=O)NCc4cccnc4)cc3)ccnc2c1]

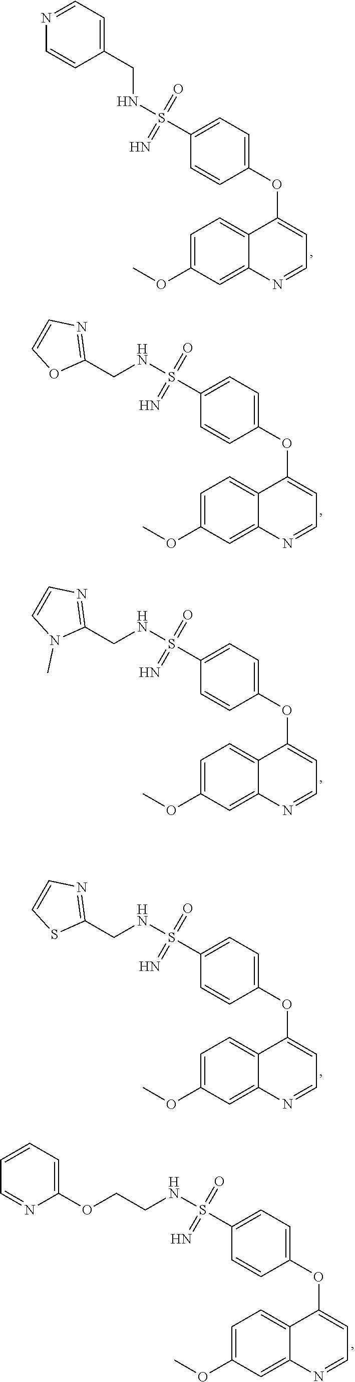 [SMILES: COc1ccc2c(Oc3ccc(S(=N)(=O)NCc4ccncc4)cc3)ccnc2c1 ; COc1ccc2c(Oc3ccc(S(=N)(=O)NCc4ncco4)cc3)ccnc2c1 ; COc1ccc2c(Oc3ccc(S(=N)(=O)NCc4nccn4C)cc3)ccnc2c1 ; COc1ccc2c(Oc3ccc(S(=N)(=O)NCc4nccs4)cc3)ccnc2c1 ; COc1ccc2c(Oc3ccc(S(=N)(=O)NCCOc4ccccn4)cc3)ccnc2c1]

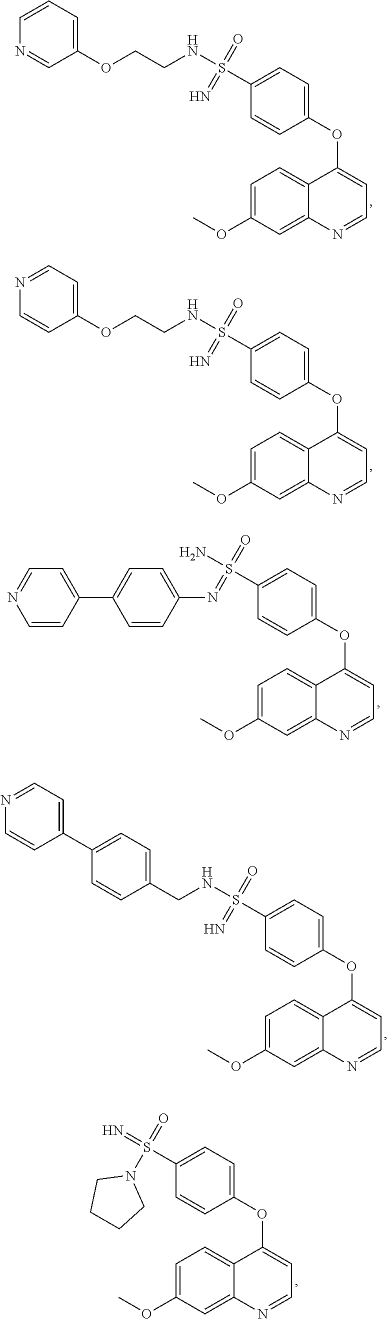 [SMILES: COc1ccc2c(Oc3ccc(S(=N)(=O)NCCOc4cccnc4)cc3)ccnc2c1 ; COc1ccc2c(Oc3ccc(S(=N)(=O)NCCOc4ccncc4)cc3)ccnc2c1 ; COc1ccc2c(Oc3ccc(S(N)(=O)=Nc4ccc(-c5ccncc5)cc4)cc3)ccnc2c1 ; COc1ccc2c(Oc3ccc(S(=N)(=O)NCc4ccc(-c5ccncc5)cc4)cc3)ccnc2c1 ; COc1ccc2c(Oc3ccc(S(=N)(=O)N4CCCC4)cc3)ccnc2c1]

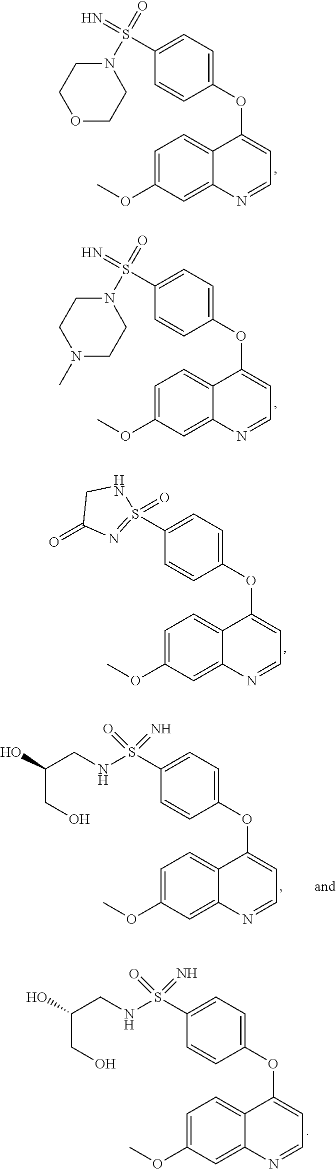

In some embodiments, the compound or salt of Formula (I), (IA), (IB), (IC), (ID), and (IE) is selected from:

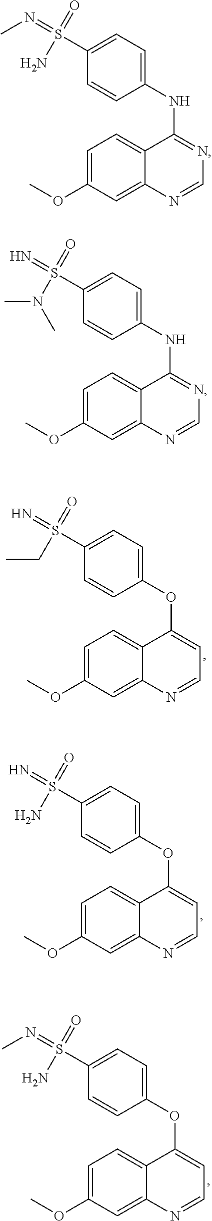

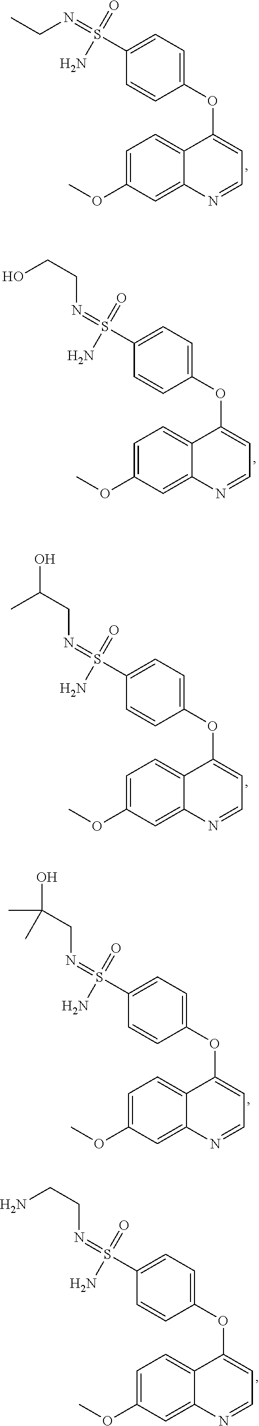 [SMILES: CCN=S(N)(=O)c1ccc(Oc2ccnc3cc(OC)ccc23)cc1 ; COc1ccc2c(Oc3ccc(S(N)(=O)=NCCO)cc3)ccnc2c1 ; COc1ccc2c(Oc3ccc(S(N)(=O)=NCC(C)O)cc3)ccnc2c1 ; COc1ccc2c(Oc3ccc(S(N)(=O)=NCC(C)(C)O)cc3)ccnc2c1 ; COc1ccc2c(Oc3ccc(S(N)(=O)=NCCN)cc3)ccnc2c1]

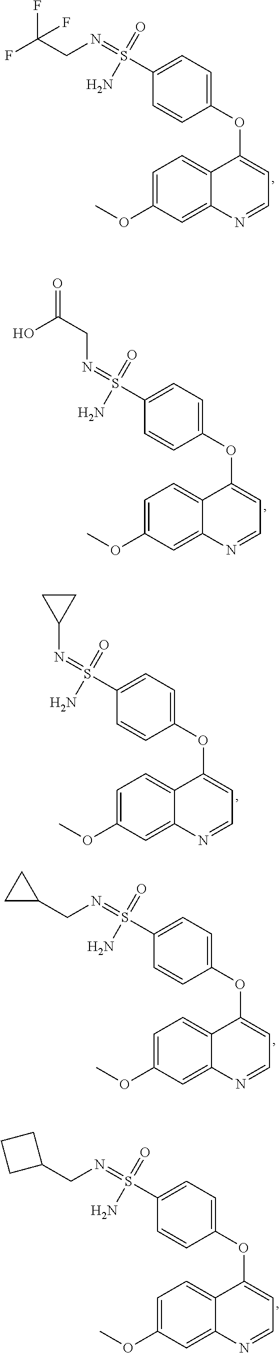 [SMILES: COc1ccc2c(Oc3ccc(S(N)(=O)=NCC(F)(F)F)cc3)ccnc2c1 ; COc1ccc2c(Oc3ccc(S(N)(=O)=NCC(=O)O)cc3)ccnc2c1 ; COc1ccc2c(Oc3ccc(S(N)(=O)=NC4CC4)cc3)ccnc2c1 ; COc1ccc2c(Oc3ccc(S(N)(=O)=NCC4CC4)cc3)ccnc2c1 ; COc1ccc2c(Oc3ccc(S(N)(=O)=NCC4CCC4)cc3)ccnc2c1]

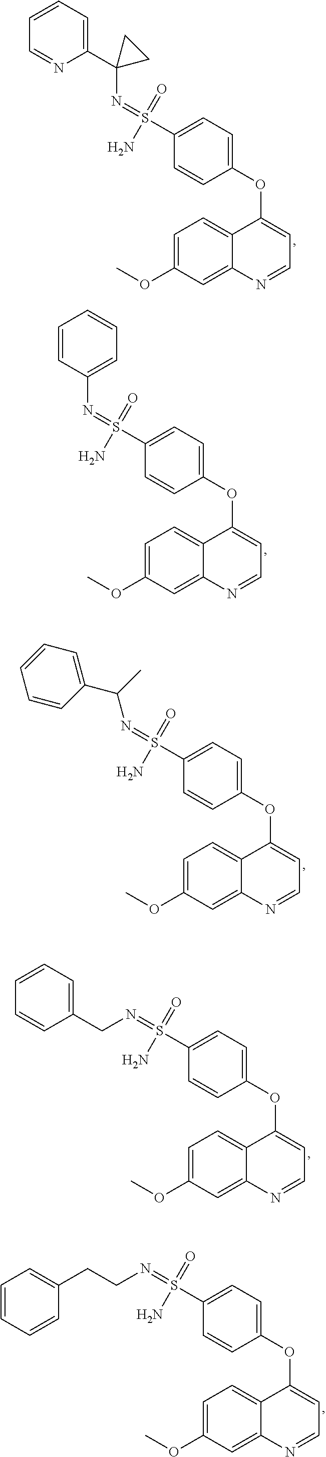 [SMILES: COc1ccc2c(Oc3ccc(S(N)(=O)=NC4(c5ccccn5)CC4)cc3)ccnc2c1 ; COc1ccc2c(Oc3ccc(S(N)(=O)=Nc4ccccc4)cc3)ccnc2c1 ; COc1ccc2c(Oc3ccc(S(N)(=O)=NC(C)c4ccccc4)cc3)ccnc2c1 ; COc1ccc2c(Oc3ccc(S(N)(=O)=NCc4ccccc4)cc3)ccnc2c1 ; COc1ccc2c(Oc3ccc(S(N)(=O)=NCCc4ccccc4)cc3)ccnc2c1]

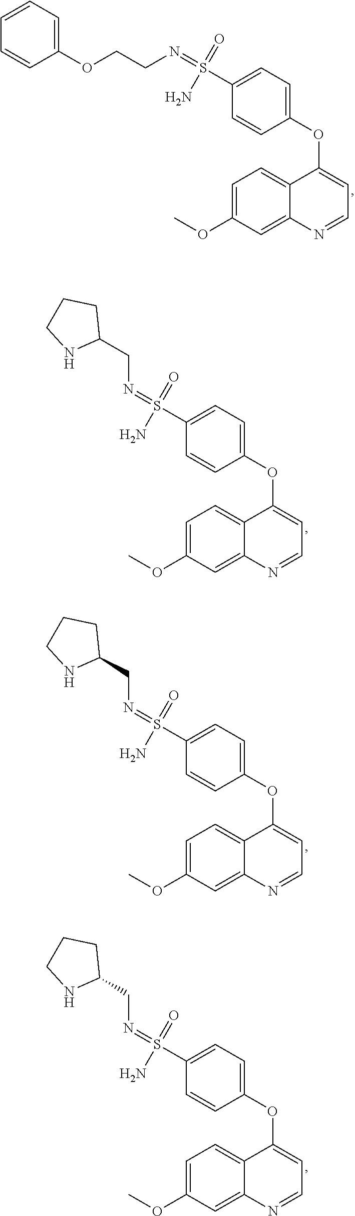 [SMILES: COc1ccc2c(Oc3ccc(S(N)(=O)=NCCOc4ccccc4)cc3)ccnc2c1 ; COc1ccc2c(Oc3ccc(S(N)(=O)=NCC4CCCN4)cc3)ccnc2c1 ; COc1ccc2c(Oc3ccc(S(N)(=O)=NC[C@@H]4CCCN4)cc3)ccnc2c1 ; COc1ccc2c(Oc3ccc(S(N)(=O)=NC[C@H]4CCCN4)cc3)ccnc2c1]

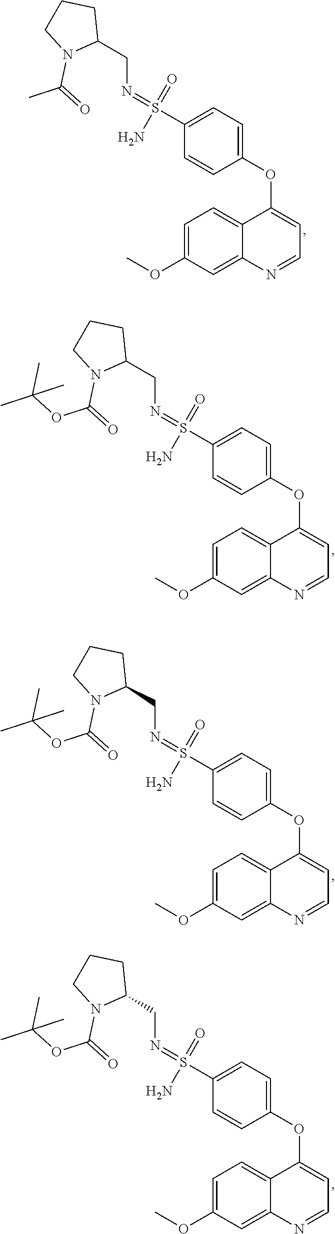 [SMILES: COc1ccc2c(Oc3ccc(S(N)(=O)=NCC4CCCN4C(C)=O)cc3)ccnc2c1 ; COc1ccc2c(Oc3ccc(S(N)(=O)=NCC4CCCN4C(=O)OC(C)(C)C)cc3)ccnc2c1 ; COc1ccc2c(Oc3ccc(S(N)(=O)=NC[C@@H]4CCCN4C(=O)OC(C)(C)C)cc3)ccnc2c1 ; COc1ccc2c(Oc3ccc(S(N)(=O)=NC[C@H]4CCCN4C(=O)OC(C)(C)C)cc3)ccnc2c1]

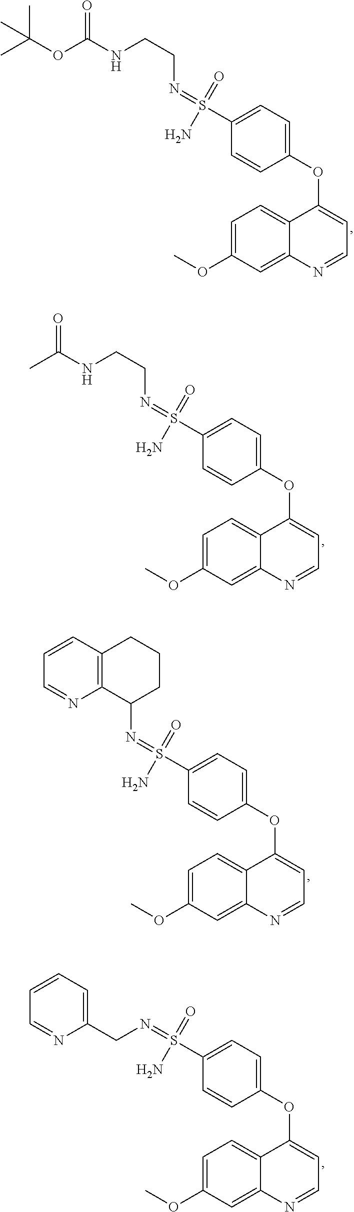 [SMILES: COc1ccc2c(Oc3ccc(S(N)(=O)=NCCNC(=O)OC(C)(C)C)cc3)ccnc2c1 ; COc1ccc2c(Oc3ccc(S(N)(=O)=NCCNC(C)=O)cc3)ccnc2c1 ; COc1ccc2c(Oc3ccc(S(N)(=O)=NC4CCCc5cccnc54)cc3)ccnc2c1 ; COc1ccc2c(Oc3ccc(S(N)(=O)=NCc4ccccn4)cc3)ccnc2c1]

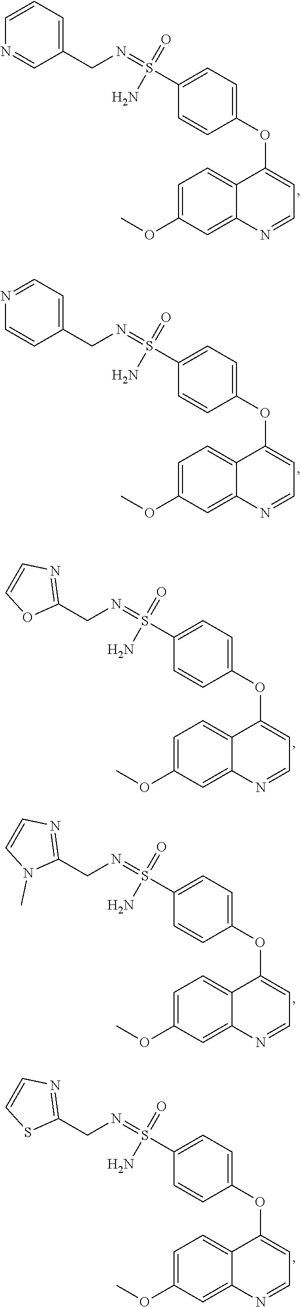 [SMILES: COc1ccc2c(Oc3ccc(S(N)(=O)=NCc4cccnc4)cc3)ccnc2c1 ; COc1ccc2c(Oc3ccc(S(N)(=O)=NCc4ccncc4)cc3)ccnc2c1 ; COc1ccc2c(Oc3ccc(S(N)(=O)=NCc4ncco4)cc3)ccnc2c1 ; COc1ccc2c(Oc3ccc(S(N)(=O)=NCc4nccn4C)cc3)ccnc2c1 ; COc1ccc2c(Oc3ccc(S(N)(=O)=NCc4nccs4)cc3)ccnc2c1]

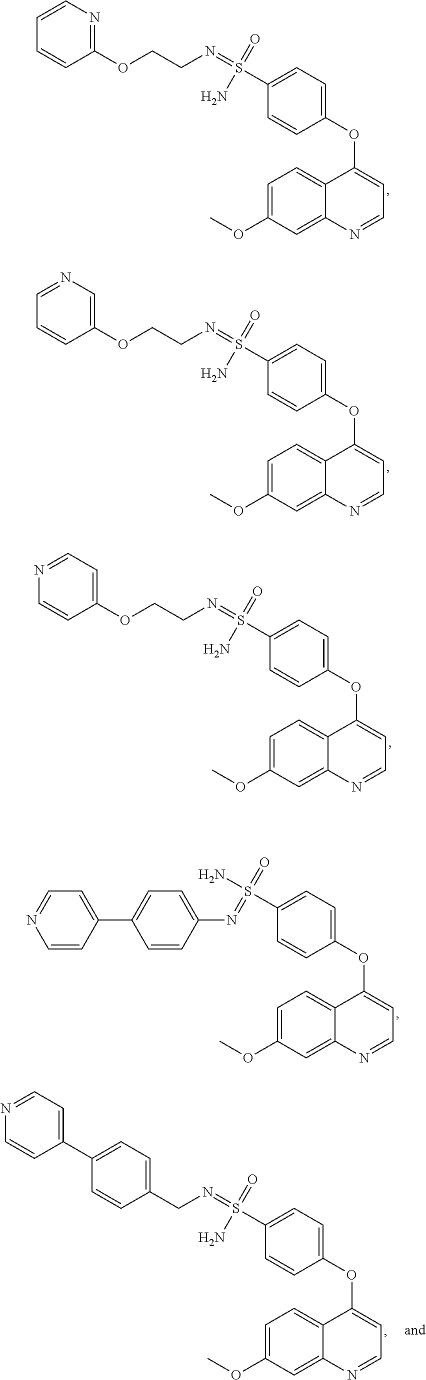 [SMILES: COc1ccc2c(Oc3ccc(S(N)(=O)=NCCOc4ccccn4)cc3)ccnc2c1 ; COc1ccc2c(Oc3ccc(S(N)(=O)=NCCOc4cccnc4)cc3)ccnc2c1 ; COc1ccc2c(Oc3ccc(S(N)(=O)=NCCOc4ccncc4)cc3)ccnc2c1 ; COc1ccc2c(Oc3ccc(S(N)(=O)=Nc4ccc(-c5ccncc5)cc4)cc3)ccnc2c1 ; COc1ccc2c(Oc3ccc(S(N)(=O)=NCc4ccc(-c5ccncc5)cc4)cc3)ccnc2c1]

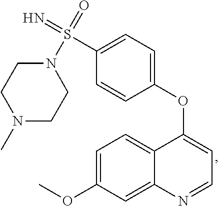 [SMILES: COc1ccc2c(Oc3ccc(S(=N)(=O)N4CCN(C)CC4)cc3)ccnc2c1]

or a tautomer thereof.

In some embodiments, the tautomer of the compound or salt of Formula (I), (IA), (IB), (IC), (ID), and (IE) is selected from:

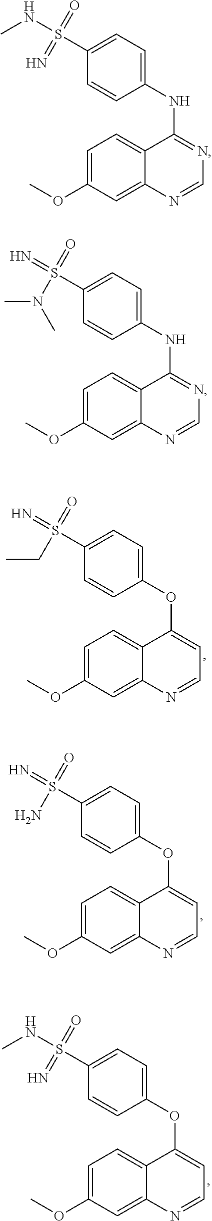

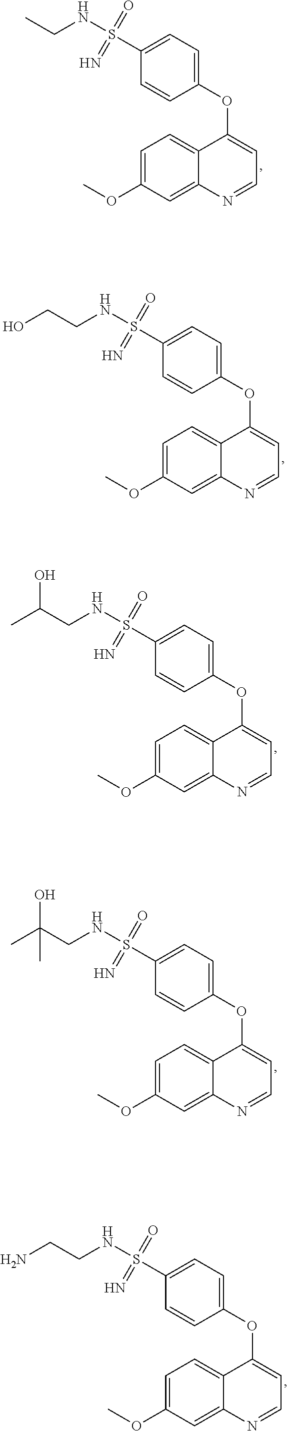 [SMILES: CCNS(=N)(=O)c1ccc(Oc2ccnc3cc(OC)ccc23)cc1 ; COc1ccc2c(Oc3ccc(S(=N)(=O)NCCO)cc3)ccnc2c1 ; COc1ccc2c(Oc3ccc(S(=N)(=O)NCC(C)O)cc3)ccnc2c1 ; COc1ccc2c(Oc3ccc(S(=N)(=O)NCC(C)(C)O)cc3)ccnc2c1 ; COc1ccc2c(Oc3ccc(S(=N)(=O)NCCN)cc3)ccnc2c1]

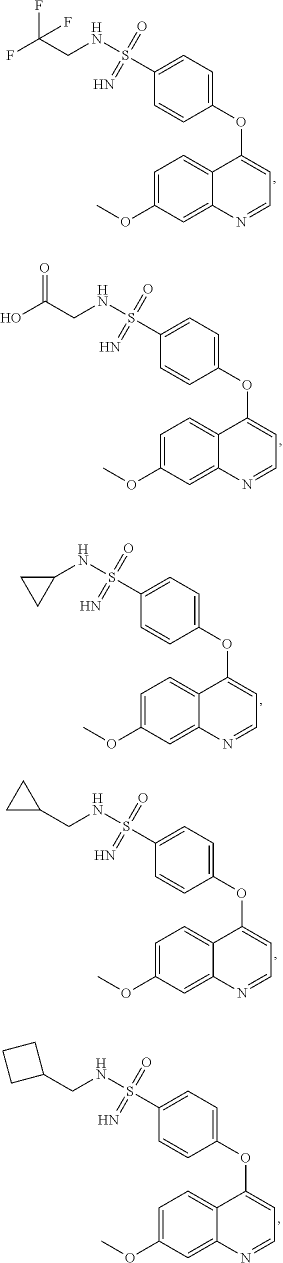 [SMILES: COc1ccc2c(Oc3ccc(S(=N)(=O)NCC(F)(F)F)cc3)ccnc2c1 ; COc1ccc2c(Oc3ccc(S(=N)(=O)NCC(=O)O)cc3)ccnc2c1 ; COc1ccc2c(Oc3ccc(S(=N)(=O)NC4CC4)cc3)ccnc2c1 ; COc1ccc2c(Oc3ccc(S(=N)(=O)NCC4CC4)cc3)ccnc2c1 ; COc1ccc2c(Oc3ccc(S(=N)(=O)NCC4CCC4)cc3)ccnc2c1]

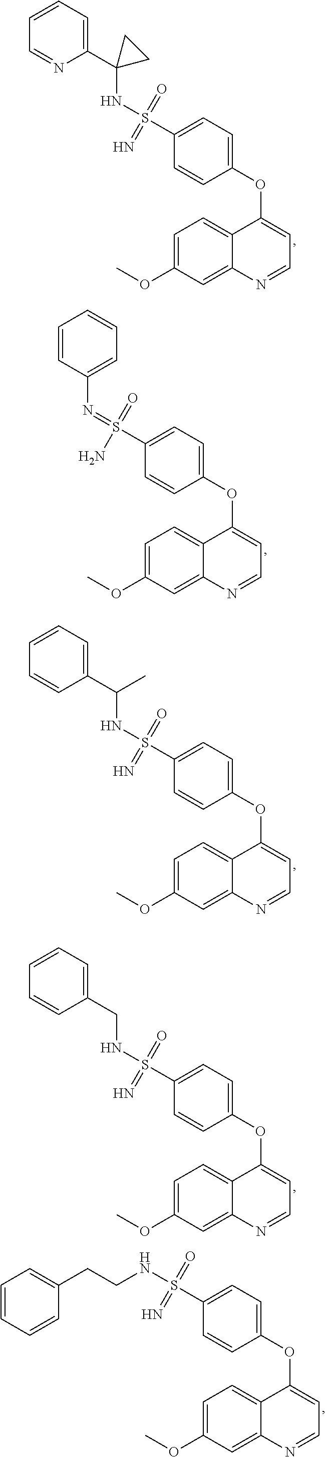

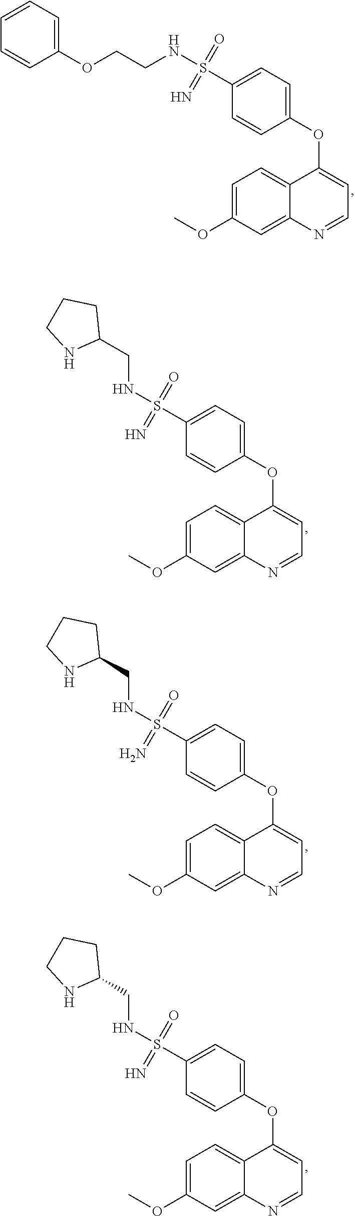 [SMILES: COc1ccc2c(Oc3ccc(S(=N)(=O)NCCOc4ccccc4)cc3)ccnc2c1 ; COc1ccc2c(Oc3ccc(S(=N)(=O)NCC4CCCN4)cc3)ccnc2c1 ; COc1ccc2c(Oc3ccc(S(=N)(=O)NC[C@@H]4CCCN4)cc3)ccnc2c1 ; COc1ccc2c(Oc3ccc(S(=N)(=O)NC[C@H]4CCCN4)cc3)ccnc2c1]

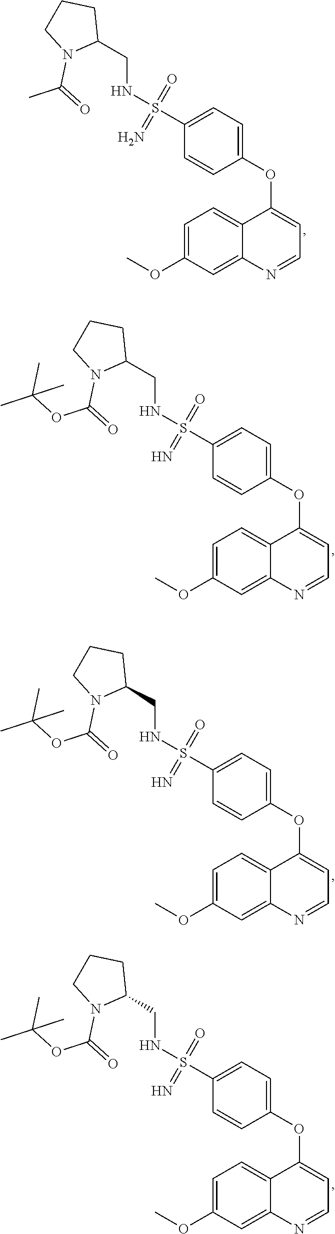 [SMILES: COc1ccc2c(Oc3ccc(S(=N)(=O)NCC4CCCN4C(C)=O)cc3)ccnc2c1 ; COc1ccc2c(Oc3ccc(S(=N)(=O)NCC4CCCN4C(=O)OC(C)(C)C)cc3)ccnc2c1 ; COc1ccc2c(Oc3ccc(S(=N)(=O)NC[C@@H]4CCCN4C(=O)OC(C)(C)C)cc3)ccnc2c1 ; COc1ccc2c(Oc3ccc(S(=N)(=O)NC[C@H]4CCCN4C(=O)OC(C)(C)C)cc3)ccnc2c1]

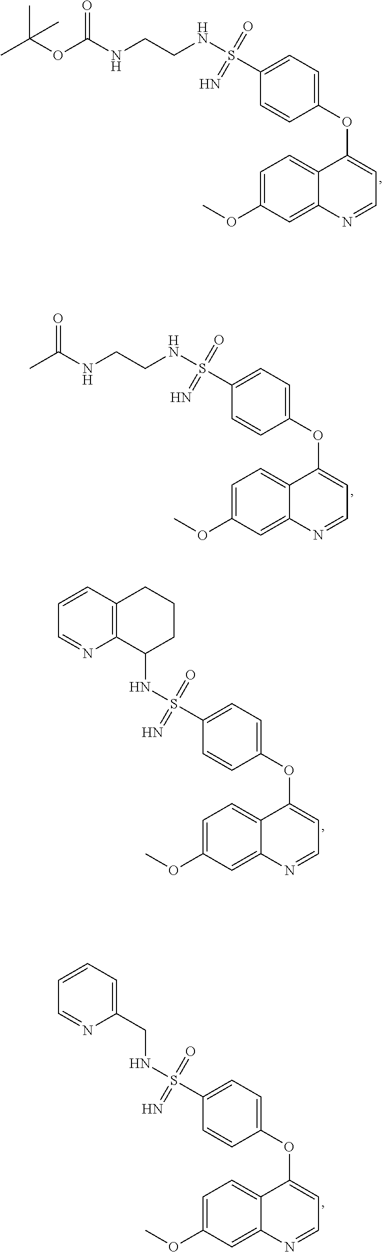 [SMILES: COc1ccc2c(Oc3ccc(S(=N)(=O)NCCNC(=O)OC(C)(C)C)cc3)ccnc2c1 ; COc1ccc2c(Oc3ccc(S(=N)(=O)NCCNC(C)=O)cc3)ccnc2c1 ; COc1ccc2c(Oc3ccc(S(=N)(=O)NC4CCCc5cccnc54)cc3)ccnc2c1 ; COc1ccc2c(Oc3ccc(S(=N)(=O)NCc4ccccn4)cc3)ccnc2c1]

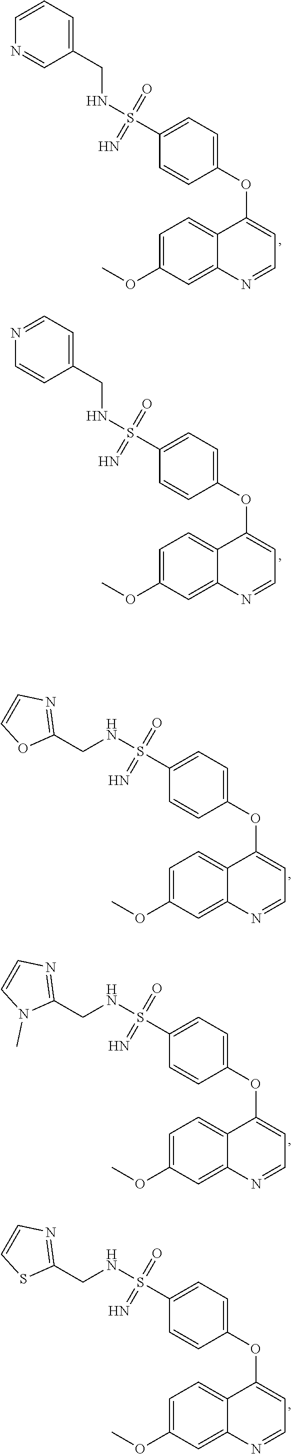 [SMILES: COc1ccc2c(Oc3ccc(S(=N)(=O)NCc4cccnc4)cc3)ccnc2c1 ; COc1ccc2c(Oc3ccc(S(=N)(=O)NCc4ccncc4)cc3)ccnc2c1 ; COc1ccc2c(Oc3ccc(S(=N)(=O)NCc4ncco4)cc3)ccnc2c1 ; COc1ccc2c(Oc3ccc(S(=N)(=O)NCc4nccn4C)cc3)ccnc2c1 ; COc1ccc2c(Oc3ccc(S(=N)(=O)NCc4nccs4)cc3)ccnc2c1]

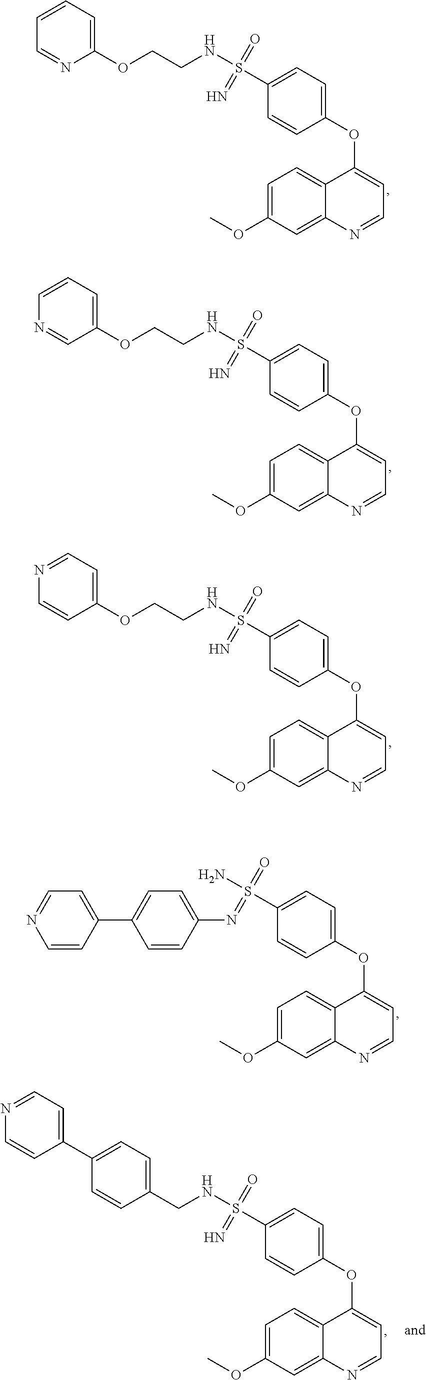 [SMILES: COc1ccc2c(Oc3ccc(S(=N)(=O)NCCOc4ccccn4)cc3)ccnc2c1 ; COc1ccc2c(Oc3ccc(S(=N)(=O)NCCOc4cccnc4)cc3)ccnc2c1 ; COc1ccc2c(Oc3ccc(S(=N)(=O)NCCOc4ccncc4)cc3)ccnc2c1 ; COc1ccc2c(Oc3ccc(S(N)(=O)=Nc4ccc(-c5ccncc5)cc4)cc3)ccnc2c1 ; COc1ccc2c(Oc3ccc(S(=N)(=O)NCc4ccc(-c5ccncc5)cc4)cc3)ccnc2c1]

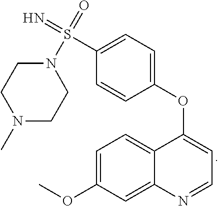 [SMILES: COc1ccc2c(Oc3ccc(S(=N)(=O)N4CCN(C)CC4)cc3)ccnc2c1]

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds or salts of Formula (I), (IA), (IB), (IC), (ID), and (IE), are intended to include all Z-, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

The compounds or salts for Formula (I), (IA), (IB), (IC), (ID), and (IE), herein may in some cases exist as diastereomers, enantiomers, or other stereoisomeric forms. The compounds presented herein include all diastereomeric, enantiomeric, and epimeric forms as well as the racemates, mixtures of diastereomers, and other mixtures thereof, to the extent they can be made by one of ordinary skill in the art by routine experimentation. Separation of stereoisomers may be performed by chromatography or by forming diastereomers and separating by recrystallization, or chromatography, or any combination thereof (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis. Furthermore, a mixture of two enantiomers enriched in one of the two can be purified to provide further optically enriched form of the major enantiomer by recrystallization and/or trituration.

In certain embodiments, compounds or salts for Formula (I), (IA), (IB), (IC), (ID), and (IE), may comprise two or more enantiomers or diatereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J. Chromatogr., 113(3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. In certain embodiments, the compounds or salts for Formula (I), (IA), (IB), (IC), (ID), and (IE), exist as tautomers. In circumstances where tautomerization is possible, a chemical equilibrium of the tautomers may exist. The exact ratio of the tautomers depends on several factors, including physical state, temperature, solvent, and pH. Some non—limiting examples of tautomeric equilibrium include:

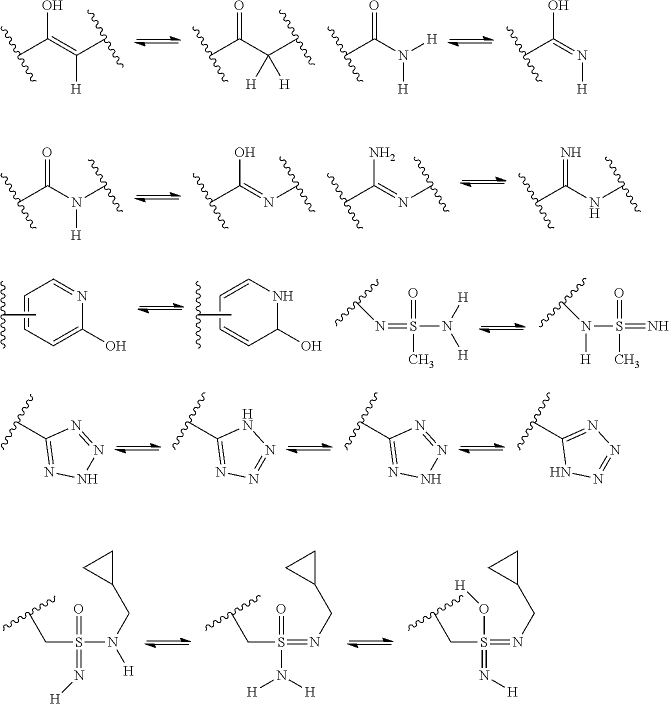

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

Included in the present disclosure are salts, particularly pharmaceutically acceptable salts, of the compounds of Formula (I), (IA), (IB), (IC), (ID), and (IE). The compounds of the present disclosure may possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The methods and compositions of Formula (I), (IA), (IB), (IC), (ID), and (IE), include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

Compounds of Formula (I), (IA), (IB), (IC), (ID), and (IE), also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of compounds represented by Formula (I), (IA), (IB), (IC), (ID), and (IE). The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

In certain embodiments, compounds or salts of Formula (I), (IA), (IB), (IC), (ID), and (IE), may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In certain embodiments, the prodrug may be converted, e.g., enzymatically or chemically, to the parent compound under the conditions within a cell. In certain embodiments, the parent compound comprises an acidic moiety, e.g., resulting from the hydrolysis of the prodrug, which may be charged under the conditions within the cell. In particular embodiments, the prodrug is converted to the parent compound once it has passed through the cell membrane into a cell. In certain embodiments, the parent compound has diminished cell membrane permeability properties relative to the prodrug, such as decreased lipophilicity and increased hydrophilicity.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Pharmaceutical Formulations

In some aspects, the present disclosure provides a pharmaceutical composition comprising a compound or salt of Formula (I), (IA), (IB), (IC) or (II) and at least one pharmaceutically acceptable excipient.

Pharmaceutical compositions can be formulated using one or more physiologically-acceptable carriers comprising excipients and auxiliaries. Formulation can be modified depending upon the route of administration chosen. Pharmaceutical compositions comprising a compound, salt or conjugate can be manufactured, for example, by lyophilizing the compound, salt or conjugate, mixing, dissolving, emulsifying, encapsulating or entrapping the conjugate. The pharmaceutical compositions can also include the compounds, salts or conjugates in a free-base form or pharmaceutically-acceptable salt form.

Methods for formulation of the conjugates can include formulating any of the compounds, salts or conjugates with one or more inert, pharmaceutically-acceptable excipients or carriers to form a solid, semi-solid, or liquid composition. Solid compositions can include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compounds, salts or conjugates can be lyophilized or in powder form for re-constitution with a suitable vehicle, e.g., sterile pyrogen-free water, before use.

Pharmaceutical compositions can comprise at least one active ingredient (e.g., a compound, salt or conjugate). The active ingredients can be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

Pharmaceutical compositions as often further can comprise more than one active compound (e.g., a compound, salt or conjugate and other agents) as necessary for the particular indication being treated. The active compounds can have complementary activities that do not adversely affect each other. For example, the composition can also comprise a chemotherapeutic agent, cytotoxic agent, cytokine, growth-inhibitory agent, anti-hormonal agent, anti-angiogenic agent, and/or cardioprotectant. Such molecules can be present in combination in amounts that are effective for the purpose intended.

The compositions and formulations can be sterilized. Sterilization can be accomplished by filtration through sterile filtration.

The compositions can be formulated for administration as an injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Injections can be formulated for bolus injection or continuous infusion. Alternatively, the compositions can be lyophilized or in powder form for reconstitution with a suitable vehicle, e.g., sterile pyrogen-free water, before use.

For parenteral administration, the compounds, salts or conjugates can be formulated in a unit dosage injectable form (e.g., solution, suspension, emulsion) in association with a pharmaceutically acceptable parenteral vehicle. Such vehicles can be inherently non-toxic, and non-therapeutic. Vehicles can be water, saline, Ringer's solution, dextrose solution, and 5% human serum albumin. Non-aqueous vehicles such as fixed oils and ethyl oleate can also be used. Liposomes can be used as carriers. The vehicle can contain minor amounts of additives such as substances that enhance isotonicity and chemical stability (e.g., buffers and preservatives).

Sustained-release preparations can also be prepared. Examples of sustained-release preparations can include semipermeable matrices of solid hydrophobic polymers that can contain the compound, salt or conjugate, and these matrices can be in the form of shaped articles (e.g., films or microcapsules). Examples of sustained-release matrices can include polyesters, hydrogels (e.g., poly(2-hydroxyethyl-methacrylate), or poly (vinyl alcohol)), polylactides, copolymers of L-glutamic acid and γ ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations can be prepared for storage by mixing a compound, salt or conjugate with a pharmaceutically acceptable carrier, excipient, and/or a stabilizer. This formulation can be a lyophilized formulation or an aqueous solution. Acceptable carriers, excipients, and/or stabilizers can be nontoxic to recipients at the dosages and concentrations used. Acceptable carriers, excipients, and/or stabilizers can include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

A compound or salt of any one of Formula (I), (IA), (IB), (IC), (ID), and (IE) may be formulated in any suitable pharmaceutical formulation. A pharmaceutical formulation of the present disclosure typically contains an active ingredient (e.g., compound or salt of any one of Formula (I), (IA), (IB), (IC), (ID), and (IE)), and one or more pharmaceutically acceptable excipients or carriers, including but not limited to: inert solid diluents and fillers, diluents, sterile aqueous solution and various organic solvents, permeation enhancers, antioxidents, solubilizers, and adjuvants.

In certain embodiments, a compound or salt of Formula (I), (IA), (IB), (IC), (ID), and (IE) is formulated with a chelating agent or other material capable of binding metal ions, such as ethylene diamine tetra acetic acid (EDTA) and its salts are capable of enhancing the stability of a compound or salt of Formula (I), (IA), (IB), (IC), (ID), and (IE).

Pharmaceutical formulations may be provided in any suitable form, which may depend on the route of administration. In some embodiments, the pharmaceutical composition disclosed herein can be formulated in dosage form for administration to a subject. In some embodiments, the pharmaceutical composition is formulated for oral, intravenous, intraarterial, aerosol, parenteral, buccal, topical, transdermal, rectal, intramuscular, subcutaneous, intraosseous, intranasal, intrapulmonary, transmucosal, inhalation, and/or intraperitoneal administration. In some embodiments, the dosage form is formulated for oral administration. For example, the pharmaceutical composition can be formulated in the form of a pill, a tablet, a capsule, an inhaler, a liquid suspension, a liquid emulsion, a gel, or a powder. In some embodiments, the pharmaceutical composition can be formulated as a unit dosage in liquid, gel, semi-liquid, semi-solid, or solid form.

The amount of compound or salt of any one of Formula (I), (IA), (IB), (IC), (ID), and (IE) will be dependent on the mammal being treated, the severity of the disorder or condition, the rate of administration, the disposition of the compound or salt of any one of Formula (I), (IA), (IB), (IC), (ID), and (IE) and the discretion of the prescribing physician.

In some embodiments, the disclosure provides a pharmaceutical composition for oral administration containing at least one compound or salt of any one of Formula (I), (IA), (IB), (IC), (ID), and (IE) and a pharmaceutical excipient suitable for oral administration. The composition may be in the form of a solid, liquid, gel, semi-liquid, or semi-solid. In some embodiments, the composition further comprises a second agent.

Pharmaceutical compositions of the disclosure suitable for oral administration can be presented as discrete dosage forms, such as hard or soft capsules, cachets, troches, lozenges, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, or dispersible powders or granules, or syrups or elixirs. Such dosage forms can be prepared by any of the methods of pharmacy, which typically include the step of bringing the active ingredient(s) into association with the carrier. In general, the composition are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient(s) in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound or salt of any one of Formula (I), (IA), (IB), (IC), (ID), and (IE) moistened with an inert liquid diluent.

In some embodiments, the disclosure provides a pharmaceutical composition for injection containing a compound or salt of any one of Formula (I), (IA), (IB), (IC), (ID), and (IE) disclosed herein and a pharmaceutical excipient suitable for injection. Components and amounts of agents in the composition are as described herein.

In certain embodiments, the compound or salt of any one of Formula (I), (IA), (IB), (IC), (ID), and (IE) may be formulated for injection as aqueous or oil suspensions, emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Pharmaceutical compositions may also be prepared from a compound or salt of any one of Formula (I), (IA), (IB), (IC), (ID), and (IE) and one or more pharmaceutically acceptable excipients suitable for transdermal, inhalative, sublingual, buccal, rectal, intraosseous, intraocular, intranasal, epidural, or intraspinal administration. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999).

### Methods of Treatment

The compounds described herein can be used in the preparation of medicaments for the prevention or treatment of diseases or conditions. In addition, a method for treating any of the diseases or conditions described herein in a subject in need of such treatment, involves administration of pharmaceutical compositions containing at least one compound described herein, or a pharmaceutically acceptable salt, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In some aspects, the present disclosure provides a method for treatment, comprising administering to a subject in need thereof an effective amount of a compound or salt of Formula (I), (IA), (IB), (IC), (ID), or (IE).

In certain embodiments, the invention provides a method of treating or preventing a disease, state or condition in a patient in need thereof comprising administering to the patient an effective amount of a compound of any one of embodiments of the invention or a pharmaceutically acceptable salt thereof. The disease, state or condition may be selected from the group consisting of viral infections, bacterial infections, cancer, tumors, and calcium-pyrophosphate disorders, e.g., psuedogout. In certain embodiments, the disease, state or condition may be a viral infection. In certain embodiments, the disease, state or condition may be a bacterial infection. In certain embodiments, the disease, state or condition may be cancer. In certain embodiments, the disease, state or condition may be a tumor. In certain embodiments, the disease, state or condition may be calcium-pyrophosphate disorder. In certain embodiments, calcium-pyrophosphate disorder is caused by calcium-pyrophosphate crystal deposition. In some embodiments, calcium-pyrophosphate disorder may be those described in Bäck, M., et al. (2019) Cardiovascular Medicine, 5(January), 1-8, Letavernier, et al. (2019) International Journal of Molecular Sciences, 20(24) and Williams, C. J. (2016) Current Opinion in Rheumatology, 28(2), 145-151.

In certain aspects, the present disclosure provides a method for immunotherapeutic treatment to a subject in need thereof In some embodiments, immunotherapy may be used to treat disorders resulting from a virus, bacteria, cancer, or tumor. In some embodiments, the present disclosure can be used as a method for immunotherapeutic treatment in a subject in need thereof, comprising administering to the subject a pharmaceutical composition comprising a compound or salt of Formula (I), (IA), (IB), (IC), (ID), or (IE), and a pharmaceutically acceptable excipient. In certain embodiments, a pharmaceutical composition comprising a compound or salt of Formula (I), (IA), (IB), (IC), (ID), or (IE), and a pharmaceutically acceptable excipient may be used as an immunological adjuvant. In some embodiments, the immunological adjuvant of the present disclosure may be used in combination with a vaccine for the treatment or prevention a disease, state or condition in a patient in need thereof. In some cases, immunological adjuvant may be as described Gutjahr, A., et al. Triggering Intracellular Receptors for Vaccine Adjuvantation. Trends in Immunology, 37(9), 573-587 (2016).

In certain embodiments, the present disclosure can be used as a method of activating an immune response to a pathogen in a subject in need thereof, comprising administering to the subject a pharmaceutical composition comprising a compound or salt of Formula (I), (IA), (IB), (IC), (ID), and (IE), and a pharmaceutically acceptable excipient.

In certain embodiments, the present disclosure can be used as a method of inhibiting ENPP1 in a subject in need thereof, comprising administering to the subject a pharmaceutical composition comprising a compound or salt of Formula (I), (IA), (IB), (IC), (ID), and (IE), and a pharmaceutically acceptable excipient.

In certain embodiments, the present disclosure can be used as a method of activating STING activity in a subject in need thereof, comprising administering to the subject a pharmaceutical composition comprising a compound or salt of Formula (I), (IA), (IB), (IC), (ID), and (IE), and a pharmaceutically acceptable excipient.

### EXAMPLES

The invention now being generally described, it will be more readily understood by reference to the following examples which are included merely for purposes of illustration of certain aspects and embodiments of the present invention, and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

Examples 1-14 show general and exemplary procedures for the preparation of the claimed ENPP1 modulators, STING modulators, or immune response modulators.

### Example 1

### General Scheme—Synthesis of Compound (1)

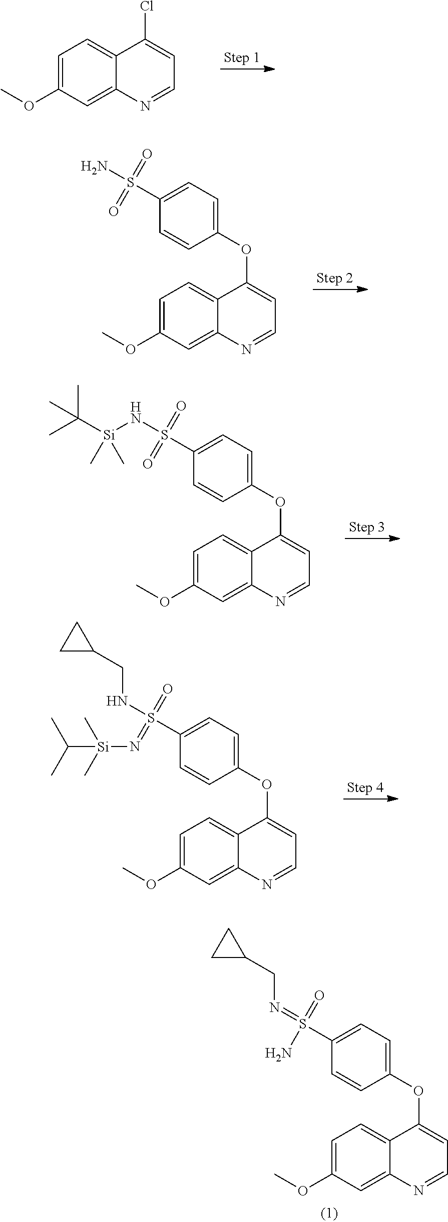 [SMILES: COc1ccc2c(Cl)ccnc2c1 ; COc1ccc2c(Oc3ccc(S(N)(=O)=O)cc3)ccnc2c1 ; COc1ccc2c(Oc3ccc(S(=O)(=O)N[Si](C)(C)C(C)(C)C)cc3)ccnc2c1 ; COc1ccc2c(Oc3ccc(S(=O)(=N[Si](C)(C)C(C)C)NCC4CC4)cc3)ccnc2c1 ; COc1ccc2c(Oc3ccc(S(N)(=O)=NCC4CC4)cc3)ccnc2c1]

### Example 2

### Exemplary Scheme—Synthesis of Compound (1)

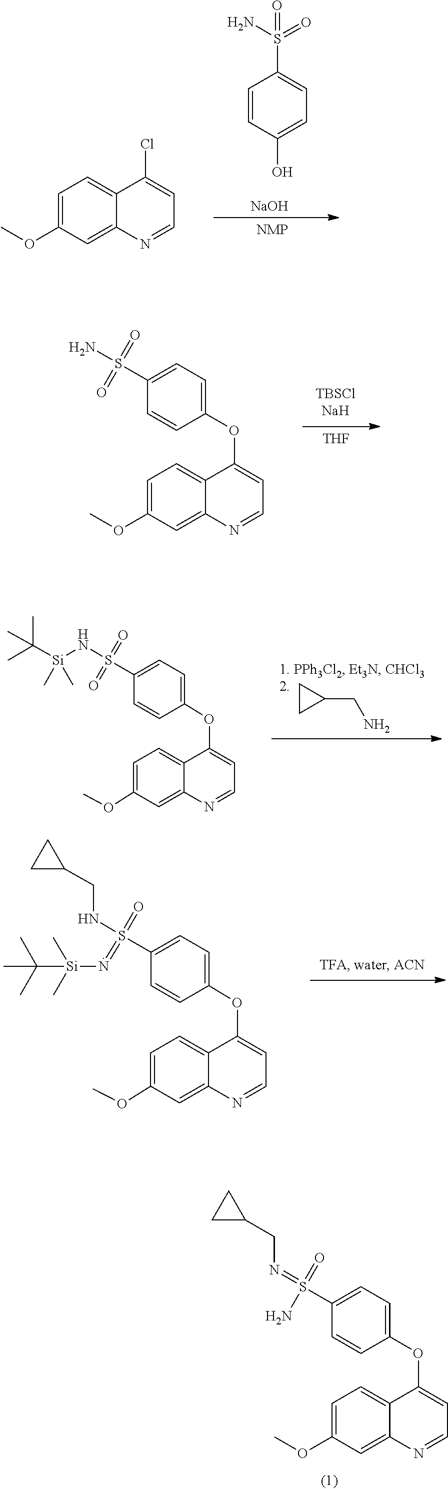

### Step 1: Preparation of 4-((7-methoxyquinolin-4-yl)oxy)benzenesulfonamide

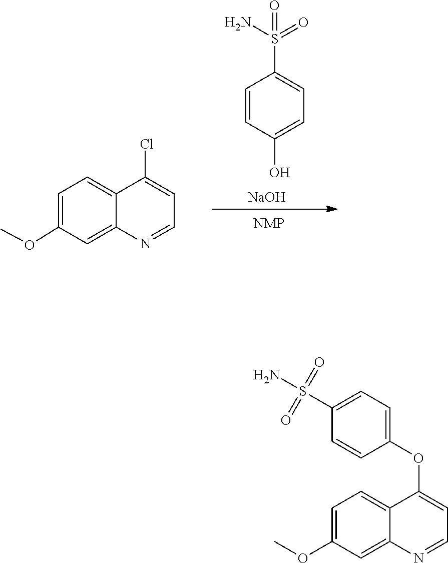

To a stirring solution of 4-hydroxybenzenesulfonamide (5.59 g, 32.3 mmol) in methanol (20 mL) was added 10M aqueous NaOH (2.6 mL, 26 mmol). The mixture was sonicated for 10 minutes and the mixture was concentrated and dried under vacuum. To the resulting solid was added 4-chloro-7-methoxyquinoline (5 g, 25.8 mmol, 1 equiv.) followed by N-methyl-2-pyrrolidone (20 mL). The mixture was heated at 150° C. for 10 h. The mixture was cooled to room temperature and diluted with water (50 ml). The resulting solution was basified to pH˜8 with aqueous 1M KH₂PO₄. The solids were filtered, washed with water, and dried by air filtration. The dried precipitate was suspended in acetonitrile (40 ml), heated to 50° C., and stirred for 15 minutes. The mixture was cooled to room temperature, filtered, and dried under vacuum. 6.4 g, LC-MS: m/z [M+H]⁺ 331.20.

### Step 2: Preparation of N-(tert-butyldimethylsilyl)-4-((7-methoxyquinolin-4-yl)oxy)benzenesulfonamide

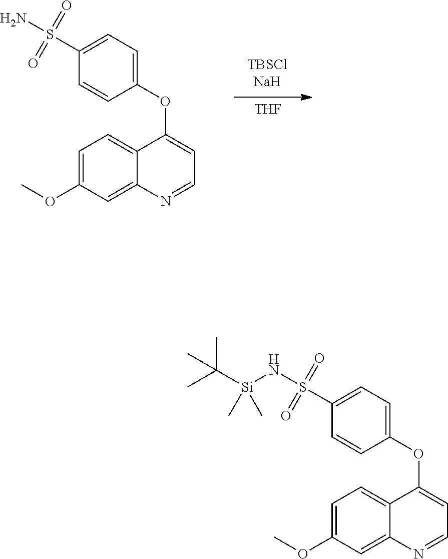 [SMILES: COc1ccc2c(Oc3ccc(S(N)(=O)=O)cc3)ccnc2c1 ; COc1ccc2c(Oc3ccc(S(=O)(=O)N[Si](C)(C)C(C)(C)C)cc3)ccnc2c1 ; "THF"]

To a suspension of 4-((7-methoxyquinolin-4-yl)oxy)benzenesulfonamide (1.8 g, 5.45 mmol) in THF (30 mL) was added portionwise sodium hydride (0.288 g, 11.99 mmol) over 15 minutes at 0° C. After stirring for 15 minutes, a solution of t-butyldimethylchlorosilane (0.985 g, 6.54 mmol) in THF (10 ml) was added dropwise over 10 mins. After stirring for 2 hours, the mixture was carefully quenched with a 2:1 mixture of 1M K₂HPO₄ and 1M KH₂PO₄ (aq, 50 mL). The layers were separated and the organic layer was partially concentrated. The crude mixture was diluted with EtOAc (40 mL) and washed with water (40 mL). The organic layer was dried with anhydrous sodium sulfate, filtered, and concentrated in vacuo. The resulting crude product was slurried in EtOAc:Et2O (1:1, 30 mL), filtered, and washed. The solids were dried under vacuum to give the title compound. 1.3 g, LC-MS: m/z [M+H]⁺ 445.20

### Step 3 and 4: Preparation of N′-(cyclopropylmethyl)-4-((7-methoxyquinolin-4-yl)oxy)benzenesulfonimidamide (1)

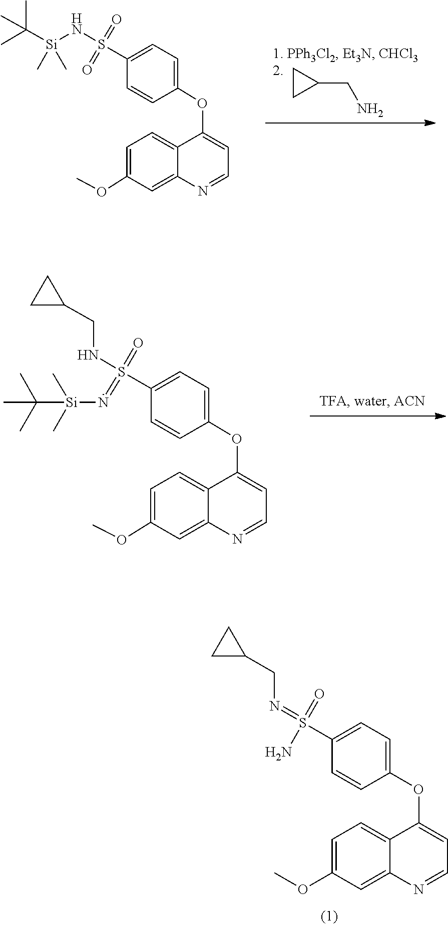

To a solution of N-(tert-butyldimethylsilyl)-4-((7-methoxyquinolin-4-yl)oxy)benzenesulfonamide (146 mg, 0.33 mmol) in chloroform (4 mL) was added dichlorotriphenylphosphorane (121 mg, 0.36 mmol) and triethylamine (69 μl, 0.49 mmol) at 0° C. The reaction mixture was stirred at 0° C. for 20 minutes. To the resulting mixture was added cyclopropylmethanamine (71 mg, 1.67 mmol) at 0° C. The mixture was warmed to 20° C. and stirred for 2 hr. The reaction mixture was quenched by addition water (20 mL) at 25° C. and extracted with ethyl acetate (3×10 mL). The combined organic layers were washed with brine, dried over Na₂SO₄, filtered, and concentrated in vacuo. The silyl group was removed with 0.1% aqueous trifluoroacetic acid in acetonitrile and the product was purified by reverse phase HPLC purification (eluent: water/acetonitrile with 0.1% TFA) to give the title compound as the trifluoroacetic acid salt. 64 mg, LC-MS: m/z [M+H]⁺ 384.1; ¹H NMR: (CD₃OD, 400 MHz) δ=8.86 (d, J=6.60 Hz, 1H), 8.52 (d, J=9.29 Hz, 1H), 8.30-8.24 (m, 2H), 7.71-7.66 (m, 2H), 7.59 (dd, J=9.35, 2.38 Hz, 1H), 7.49 (d, J=2.32 Hz, 1H), 6.98 (d, J=6.60 Hz, 1H), 4.10 (s, 3H), 3.09-2.92 (m, 2H), 1.04-0.87 (m, 1H), 0.53-0.49 (m, 2H), 0.32-0.09 (m, 2H)

### Example 3

### General Scheme—Synthesis of Compound (2)

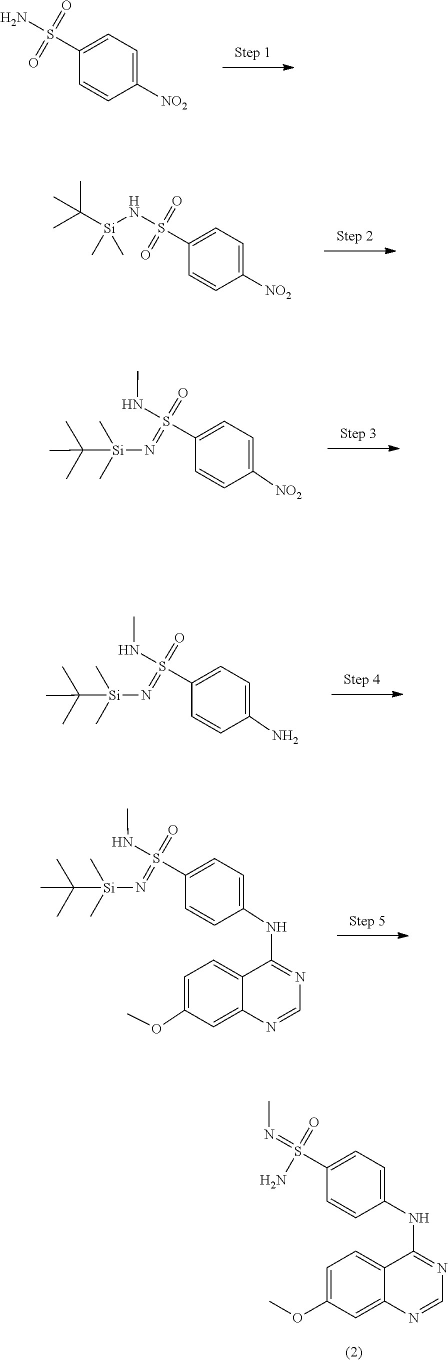 [SMILES: NS(=O)(=O)c1ccc([N+](=O)[O-])cc1 ; CC(C)(C)[Si](C)(C)NS(=O)(=O)c1ccc([N+](=O)[O-])cc1 ; CNS(=O)(=N[Si](C)(C)C(C)(C)C)c1ccc([N+](=O)[O-])cc1 ; CNS(=O)(=N[Si](C)(C)C(C)(C)C)c1ccc(N)cc1 ; CNS(=O)(=N[Si](C)(C)C(C)(C)C)c1ccc(Nc2ncnc3cc(OC)ccc23)cc1 ; CN=S(N)(=O)c1ccc(Nc2ncnc3cc(OC)ccc23)cc1]

### Example 4

### Exemplary Scheme—Synthesis of Compound (2)

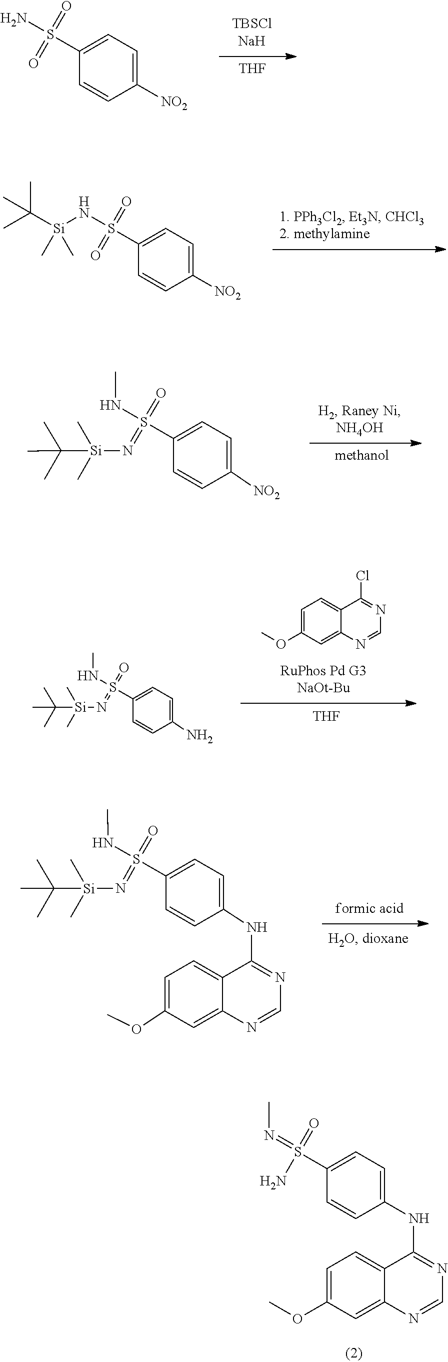

### Step 1: Preparation of N-(tert-butyldimethylsilyl)-4-nitrobenzenesulfonamide

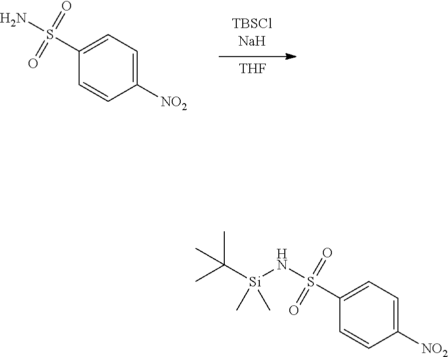 [SMILES: NS(=O)(=O)c1ccc([N+](=O)[O-])cc1 ; CC(C)(C)[Si](C)(C)NS(=O)(=O)c1ccc([N+](=O)[O-])cc1 ; "THF"]

To a solution of sodium hydride (1.48 g, 37.1 mmol, 60% dispersion in oil) in tetrahydrofuran (20 mL) was added dropwise the solution of 4-nitrobenzenesulfonamide (3 g, 14.84 mmol) in tetrahydrofuran (50 mL) at 5° C. over 2 minutes. After stirring for 30 min, t-butyldimethylchlorosilane (2.68 g, 17.8 mmol) in tetrahydrofuran (THF, 10 mL) was added dropwise at 5° C. The resulting mixture was stirred at 20° C. for 12 h. The reaction mixture was concentrated under reduced pressure and the resulting crude mixture was diluted with water (80 mL) and extracted with ethyl acetate (2×80 mL). The combined organic layers was washed with water (2×100 mL), dried over Na₂SO₄, filtered, and concentrated under reduced pressure. The crude product was stirred with petroleum ether (20 mL) for 10 minutes, filtered, and dried under vacuum to give the title compound. 4.6 g, LC-MS: m/z [M+H]⁺ 317.1.

### Step 2: Preparation of N′-(tert-butyldimethylsilyl)-N-methyl-4-nitrobenzenesulfonimidamide

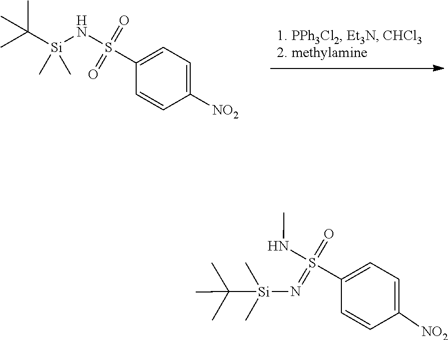 [SMILES: CC(C)(C)[Si](C)(C)NS(=O)(=O)c1ccc([N+](=O)[O-])cc1 ; CNS(=O)(=N[Si](C)(C)C(C)(C)C)c1ccc([N+](=O)[O-])cc1]

To a solution of dichlorotriphenylphosphorane (496 mg, 1.49 mmol) in chloroform (15 mL) was added triethylamine (412 mg, 4.06 mmol). After stirring the mixture at 20° C. for 10 minutes, the mixture was cooled to 0° C. and N-(tert-butyldimethylsilyl)-4-nitrobenzenesulfonamide (429 mg, 1.36 mmol) was added. After stirring at 0° C. for 30 minutes, methylamine (185 mg, 3.58 mmol, 30%) was added and the mixture was stirred at 0° C. for 30 minutes. The reaction was repeated 3 times and combined. The resulting mixture was washed with water, organic layer separated, dried with Na₂SO₄, filtered, and concentrated. The product was purified by silica chromatography using ethyl acetate in petroleum ether to give the title compound. 600 mg, LC-MS: m/z [M+H]⁺ 330.1.

### Step 3: Preparation of 4-amino-N′-(tert-butyldimethylsilyl)-N-methylbenzenesulfonimidamide

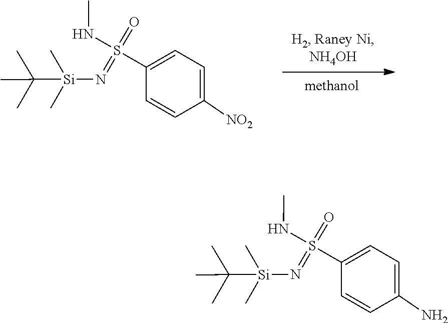 [SMILES: CNS(=O)(=N[Si](C)(C)C(C)(C)C)c1ccc([N+](=O)[O-])cc1 ; CNS(=O)(=N[Si](C)(C)C(C)(C)C)c1ccc(N)cc1]

To a solution of N′-(tert-butyldimethylsilyl)-N-methyl-4-nitrobenzenesulfonimidamide (200 mg, 0.607 mmol) in methanol (2 mL) was added concentrated ammonium hydroxide (4.2 uL) and Raney-Ni (133.33 mg, 1.56 mmol, 2.56 eq) under nitrogen gas. The reaction vessel was purged with hydrogen gas and stirred for 30 minutes at ambient temperature. The reaction mixture was filtered and concentrated under reduced pressure to give the title compound which was taken to the next step without further purification. 250 mg, LC-MS: m/z [M+H]⁺ 300.1.

### Step 4: Preparation of N′-(tert-butyldimethylsilyl)-4-((7-methoxyquinazolin-4-yl)amino)-N-methylbenzenenesulfonimidamide

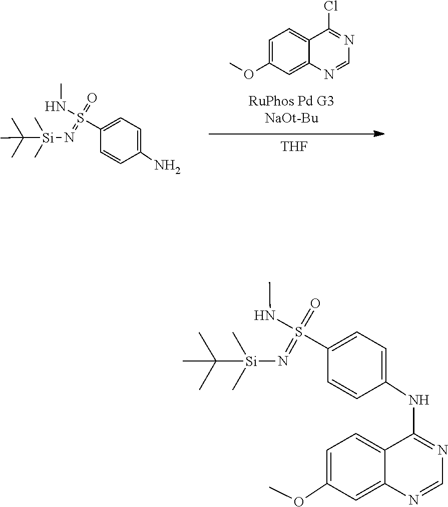 [SMILES: COc1ccc2c(Cl)ncnc2c1 ; CNS(=O)(=N[Si](C)(C)C(C)(C)C)c1ccc(N)cc1 ; CNS(=O)(=N[Si](C)(C)C(C)(C)C)c1ccc(Nc2ncnc3cc(OC)ccc23)cc1 ; "THF"]

A mixture of 4-amino-N′-(tert-butyldimethylsilyl)-N-methylbenzenesulfonimidamide (100 mg, 334 umol), 4-chloro-7-methoxyquinazoline (71 mg, 367 umol, 1.1 eq), sodium tert-butoxide (64 mg, 668 umol), and RuPhos Pd G3 (27 mg, 33.4 umol) in THF (3 mL) was degassed and purged with nitrogen. The mixture was stirred at 72° C. for 12 hours under nitrogen gas. The reaction mixture was filtered and concentrated under vacuum and title compound was taken to the next step without further purification.

### Step 5: Preparation of N′-(tert-butyldimethylsilyl)-4-((7-methoxyquinazolin-4-yl)amino)-N-methylbenzenesulfonimidamide (2)

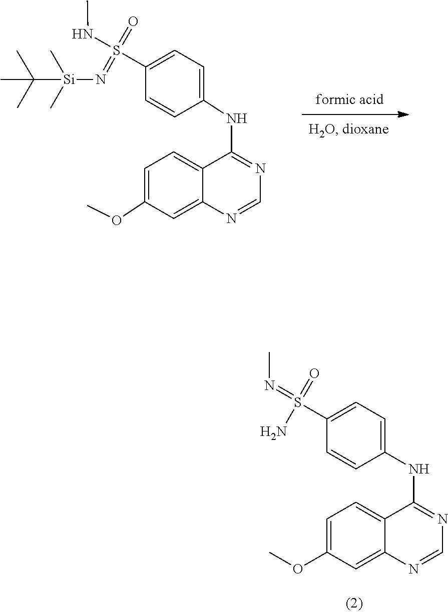 [SMILES: CNS(=O)(=N[Si](C)(C)C(C)(C)C)c1ccc(Nc2ncnc3cc(OC)ccc23)cc1 ; CN=S(N)(=O)c1ccc(Nc2ncnc3cc(OC)ccc23)cc1]

The crude mixture containing N′-(tert-butyldimethylsilyl)-4-((7-methoxyquinazolin-4-yl)amino)-N-methylbenzenesulfonimidamide from the previous reaction was dissolved dioxane (3 mL), water (1 mL), and formic acid (157 mg, 3.28 mmol). The mixture was stirred at 20° C. for 30 minutes. The reaction mixture was filtered, concentrated under reduced pressure, and the product was purified by reverse phase HPLC purification (eluent: water/acetonitrile with 10 mM NH₄HCO₃) to give the title compound. 7.5 mg, LC-MS: m/z [M+H]⁺ 344.1; ¹H NMR (DMSO-d6, 400 MHz) δ=8.56 (s, 1H), 8.42 (d, J=9.3 Hz, 1H), 8.00 (d, J=8.8 Hz, 2H), 7.81 (d, J=8.6 Hz, 2H), 7.26 (dd, J=2.5, 9.2 Hz, 1H), 7.18 (d, J=2.4 Hz, 1H), 3.90 (s, 3H), 2.35 (s, 3H)

### Example 5

### General Scheme—Synthesis of Compound (3)

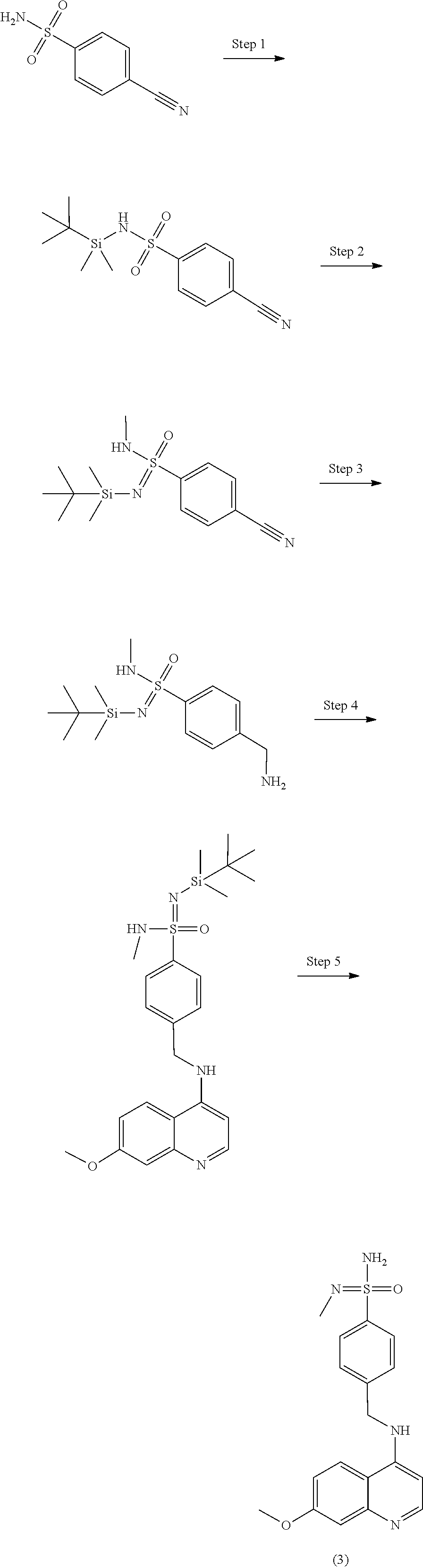 [SMILES: N#Cc1ccc(S(N)(=O)=O)cc1 ; CC(C)(C)[Si](C)(C)NS(=O)(=O)c1ccc(C#N)cc1 ; CNS(=O)(=N[Si](C)(C)C(C)(C)C)c1ccc(C#N)cc1 ; CNS(=O)(=N[Si](C)(C)C(C)(C)C)c1ccc(CN)cc1 ; CNS(=O)(=N[Si](C)(C)C(C)(C)C)c1ccc(CNc2ccnc3cc(OC)ccc23)cc1 ; CN=S(N)(=O)c1ccc(CNc2ccnc3cc(OC)ccc23)cc1]

### Example 6

### Exemplary Scheme—Synthesis of Compound (3)

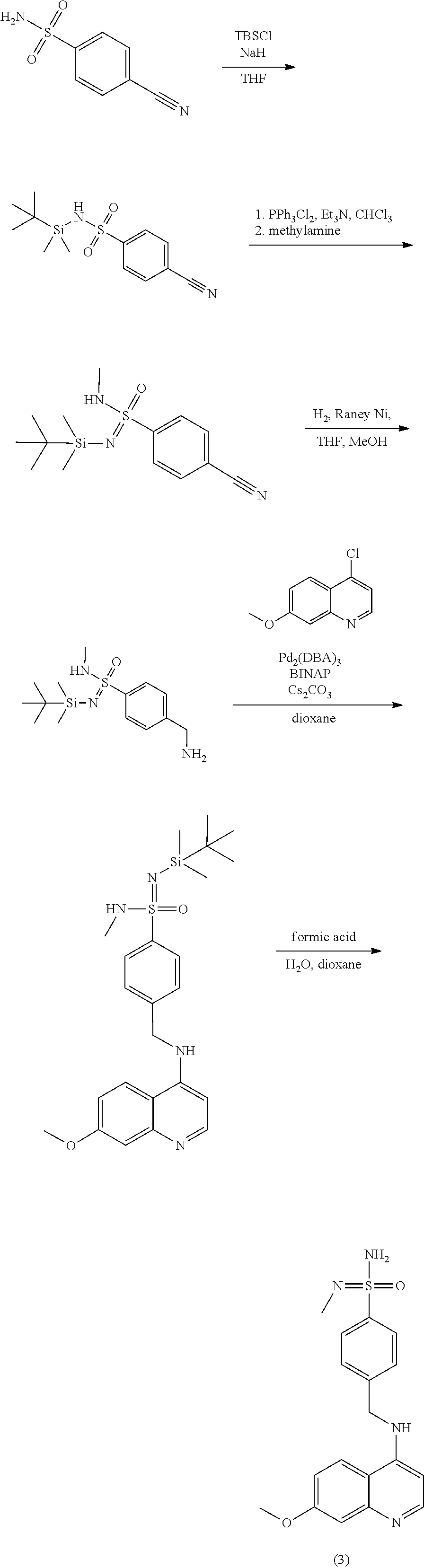

### Step 1: Preparation of N-(tert-butyldimethylsilyl)-4-cyanobenzenesulfonamide

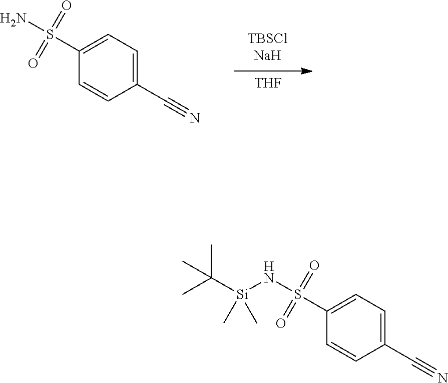 [SMILES: N#Cc1ccc(S(N)(=O)=O)cc1 ; CC(C)(C)[Si](C)(C)NS(=O)(=O)c1ccc(C#N)cc1 ; "THF"]

To a solution of 4-cyanobenzenesulfonamide (1.0 g, 5.49 mmol) in tetrahydrofuran (20 mL) was added sodium hydride (329 mg, 8.23 mmol, 60% dispersion in oil) at 0° C. The reaction mixture was stirred at 0° C. for 30 minutes. To the reaction mixture was added tert-butyldimethylsilyl chloride (910 mg, 6.04 mmol) at 0° C. The reaction mixture was stirred at 0° C. for 30 minutes and warmed 20° C. and stirred for an additional 2 hours. The reaction was quenched with water (10 mL) and stirred for 10 minutes. The resulting mixture was extracted with ethyl acetate (100 mL) and water (50 mL). The aqueous layer was back-extracted with ethyl acetate (50 mL×2). The organic layers were combined, washed with brine, dried over Na₂SO₄, filtered, and concentrated in vacuo. The product was purified by silica chromatography using ethyl acetate in petroleum ether to give the title compound. 1.1 g, LC-MS: m/z [M+H]⁺ 297.1

### Step 2: Preparation of N′-(tert-butyldimethylsilyl)-4-cyano-N-methylbenzenesulfonimidamide

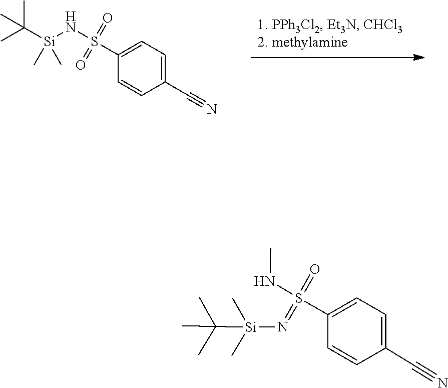 [SMILES: CC(C)(C)[Si](C)(C)NS(=O)(=O)c1ccc(C#N)cc1 ; CNS(=O)(=N[Si](C)(C)C(C)(C)C)c1ccc(C#N)cc1]

N′-(tert-butyldimethylsilyl)-4-cyano-N-methylbenzenesulfonimidamide was prepared using the procedure described above in Example 4 using N-(tert-butyldimethylsilyl)-4-cyanobenzenesulfonamide (1.1 g, 3.71 mmol) in place of N-(tert-butyldimethylsilyl)-4-nitrobenzenesulfonamide. The product was purified by silica chromatography using ethyl acetate in petroleum ether to give the title compound. 750 mg; LC-MS: m/z [M+H]⁺ 310.1

### Step 3: Preparation of 4-(aminomethyl)-N′-(tert-butyldimethylsilyl)-N-methylbenzenesulfonimidamide

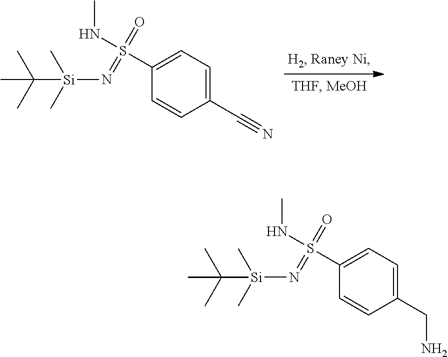 [SMILES: CNS(=O)(=N[Si](C)(C)C(C)(C)C)c1ccc(C#N)cc1 ; CNS(=O)(=N[Si](C)(C)C(C)(C)C)c1ccc(CN)cc1]

To a solution of N′-(tert-butyldimethylsilyl)-4-cyano-N-methylbenzenesulfonimidamide (300 mg, 0.97 mmol) in tetrahydrofuran (25 mL) and methanol (25 mL) was added concentrated ammonium hydroxide (0.1 mL) and Raney-Ni (300 mg, 3.51 mmol) at 20° C. The reaction mixture was stirred at 20° C. for 0.5 hours under H₂. The solids were filtered and the filter cake was washed with ethyl acetate, and the filtrate was concentrated under vacuum to give the crude product (300 mg, yield 98.71%) which was used into the next step without further purification. LC-MS: m/z [M+H]⁺ 314.1

### Step 4: Preparation of N′-(tert-butyldimethylsilyl)-4-(((7-methoxyquinolin-4-yl)amino)methyl)-N-methylbenzenesulfonimidamide

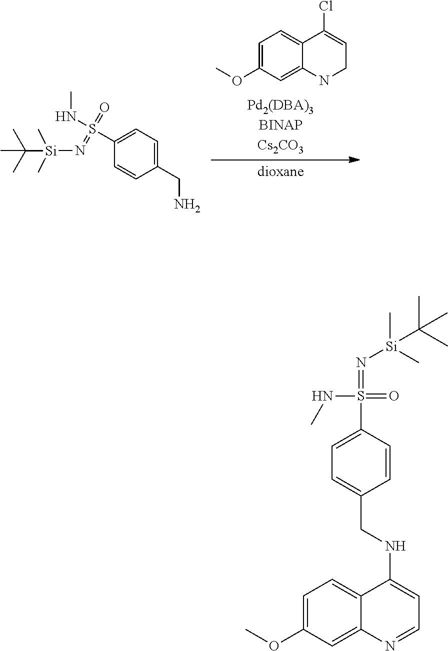 [SMILES: COc1ccc2c(c1)NCC=C2Cl ; CNS(=O)(=N[Si](C)(C)C(C)(C)C)c1ccc(CN)cc1 ; CNS(=O)(=N[Si](C)(C)C(C)(C)C)c1ccc(CNc2ccnc3cc(OC)ccc23)cc1]

To a solution of 4-chloro-7-methoxyquinoline (61.8 mg, 319 umol) in dioxane (3 mL) was added Cs₂CO₃ (259 mg, 797 umol), BINAP (29.8 mg, 47.8 umol), 4-(aminomethyl)-N′-(tert-butyldimethylsilyl)-N-methylbenzenesulfonimidamide (100 mg, 319 umol), and Pd₂(dba)₃ (18 mg, 31.9 umol). The reaction mixture was stirred at 120° C. for 16 h under nitrogen gas. The resulting mixture was partitioned between ethyl acetate (15 mL) and water (10 mL). The aqueous layer was back-extracted with ethyl acetate (10 mL×3). The organic layers were combined, washed with brine, dried over Na₂SO₄, filtered, and concentrated in vacuo. The product was purified by silica chromatography using methanol in ethyl acetate to give the title compound. 100 mg, LC-MS: m/z [M+H]⁺ 471.3

### Step 5: Preparation of 4-(((7-methoxyquinolin-4-yl)amino)methyl)-N-methylbenzenesulfonimidamide (3)

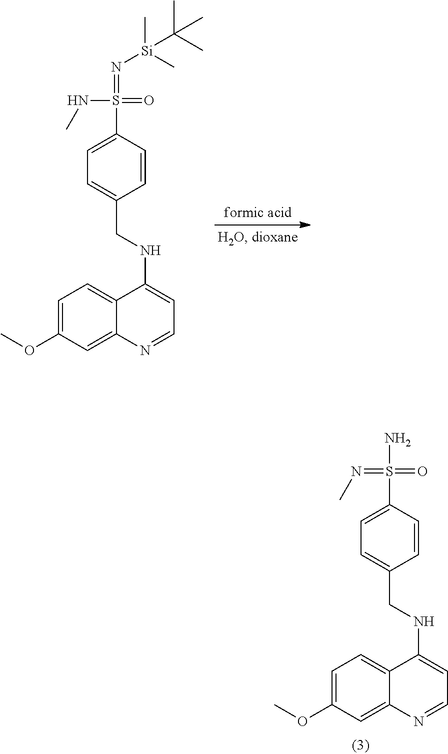 [SMILES: CNS(=O)(=N[Si](C)(C)C(C)(C)C)c1ccc(CNc2ccnc3cc(OC)ccc23)cc1 ; CN=S(N)(=O)c1ccc(CNc2ccnc3cc(OC)ccc23)cc1]

4-(((7-methoxyquinolin-4-yl)amino)methyl)-N-methylbenzenesulfonimidamide (3) was prepared using the procedure described above in Example 2 using N′-(tert-butyldimethylsilyl)-4-(((7-methoxyquinolin-4-yl)amino)methyl)-N-methylbenzenesulfonimidamide (300 mg, 0.97 mmol) in place of N′-(tert-butyldimethylsilyl)-4-((7-methoxyquinazolin-4-yl)amino)-N-methylbenzenesulfonimidamide. The the product was purified by reverse phase HPLC purification (eluent: water/acetonitrile) to give the title compound. 27 mg, LC-MS: m/z [M+H]⁺ 357.2; ¹H NMR: (DMSO-d6, 400 MHz) δ=8.25-8.17 (m, 2H), 7.93 (br t, J=6.0 Hz, 1H), 7.78 (d, J=8.4 Hz, 2H), 7.52 (d, J=8.4 Hz, 2H), 7.17 (d, J=2.4 Hz, 1H), 7.11 (dd, J=2.4, 9.2 Hz, 1H), 6.20 (d, J=5.2 Hz, 1H), 4.60 (br d, J=6.0 Hz, 2H), 3.87 (s, 3H), 2.32 (s, 3H).

### Example 7

### General Scheme—Synthesis of Compound (4)

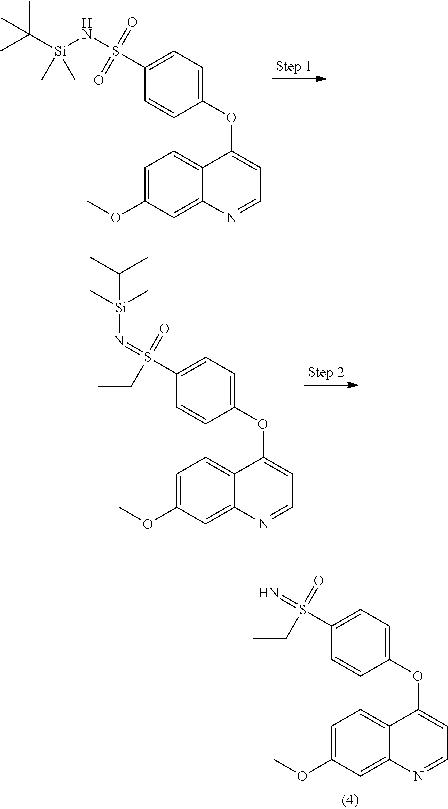 [SMILES: COc1ccc2c(Oc3ccc(S(=O)(=O)N[Si](C)(C)C(C)(C)C)cc3)ccnc2c1 ; CCS(=O)(=N[Si](C)(C)C(C)C)c1ccc(Oc2ccnc3cc(OC)ccc23)cc1 ; CCS(=N)(=O)c1ccc(Oc2ccnc3cc(OC)ccc23)cc1]

### Example 8

### Exemplary Scheme—Synthesis of Compound (4)

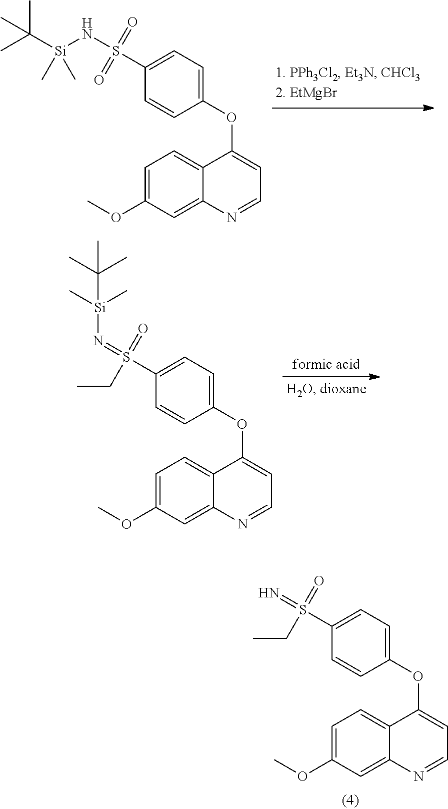

### Step 1: Preparation of ((tert-butyldimethylsilyl)imino)(ethyl)(4-((7-methoxyquinolin-4-yl)oxy)phenyl)-λ6-sulfanone

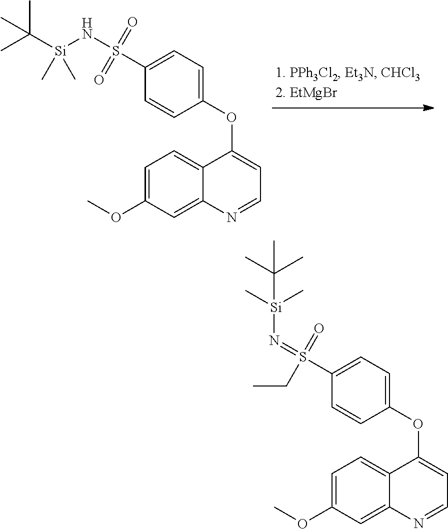

To a solution of N-(tert-butyldimethylsilyl)-4-((7-methoxyquinolin-4-yl)oxy)benzenesulfonamide (200 mg, 0.45 mmol) in chloroform (10 mL) was added dichlorotriphenylphosphorane (222 mg, 0.67 mmol), triethylamine (187 μL, 1.35 mmol). After stirring for 30 minutes, 1M ethylmagnesium bromide in tetrahydrofuran (0.9 mL, 1.8 mmol) was added. The reaction mixture was stirred at 20° C. for 1 hour under nitrogen. The mixture was concentrated under reduced pressure. The residue was diluted with methanol (2 mL) and purified by preparative thin layer chromatography using ethyl acetate. 20 mg, LC-MS: m/z [M+H]⁺ 457.2

### Step 2: Preparation of ethyl(imino)(4-((7-methoxyquinolin-4-yl)oxy)phenyl)-λ6-sulfanone (4)

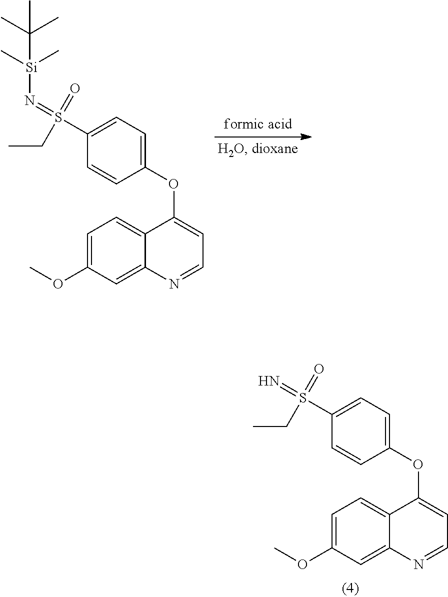 [SMILES: CCS(=O)(=N[Si](C)(C)C(C)(C)C)c1ccc(Oc2ccnc3cc(OC)ccc23)cc1 ; CCS(=N)(=O)c1ccc(Oc2ccnc3cc(OC)ccc23)cc1]

To a solution of ((tert-butyldimethylsilyl)imino)(ethyl)(4-((7-methoxyquinolin-4-yl)oxy)phenyl)-λ6-sulfanone (20 mg, 44 umol) in dichloromethane (1 mL) was added formic acid (12 mg, 132 umol). The mixture was stirred at 20° C. for 1 hour. The reaction mixture was concentrated under reduced pressure and purified by reverse phase HPLC purification (eluent: water/acetonitrile with 10 mM NH₄HCO₃) to give the title compound. 1.4 mg, LC-MS: m/z [M+H]⁺ 343.1; ¹H NMR: (DMSO-d6, 400 MHz) δ=8.70 (d, J=5.1 Hz, 1H), 8.13 (d, J=9.0 Hz, 1H), 7.97 (d, J=8.7 Hz, 2H), 7.48-7.40 (m, 3H), 7.31 (dd, J=2.5, 9.2 Hz, 1H), 6.71 (d, J=5.1 Hz, 1H), 3.94 (s, 3H), 3.16 (q, J=7.3 Hz, 2H), 1.10 (t, J=7.3 Hz, 3H).

### Example 9

### Exemplary Scheme—Synthesis of Compound (5) and (6)

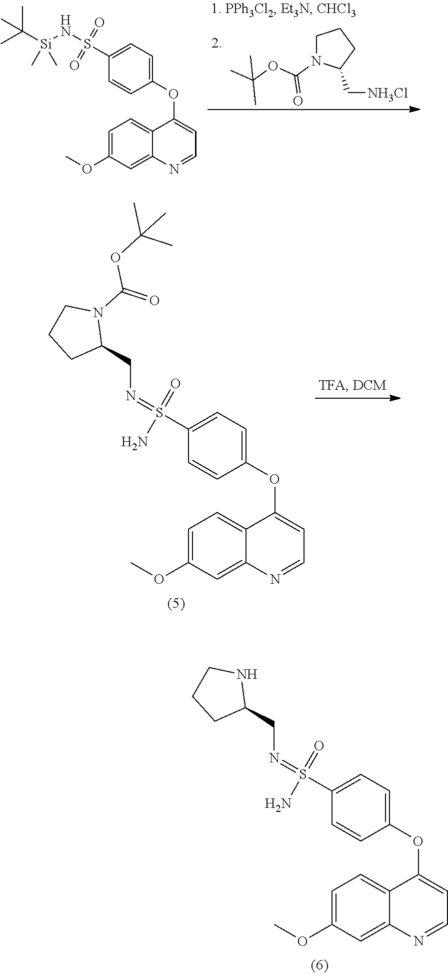

### Step 1: Preparation of tert-butyl (2R)-2-(((amino(4-((7-methoxyquinolin-4-yl)oxy)phenyl)(oxo)-λ6-sulfaneylidene)amino)methyl)pyrrolidine-1-carboxylate (5)

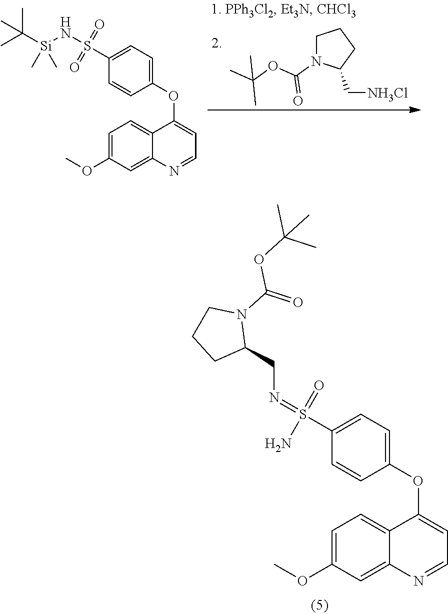

tert-butyl (2R)-2-(((amino(4-((7-methoxyquinolin-4-yl)oxy)phenyl)(oxo)-λ6-sulfaneylidene)amino)methyl)pyrrolidine-1-carboxylate was prepared using the procedure described above in Example 1 using tert-butyl (S)-2-(aminomethyl)pyrrolidine-1-carboxylate hydrochloride (260 mg, 1.3 mmol, 3 equiv.) in place of cyclopropylmethanamine. The product was purified by silica chromatography using ethyl acetate in petroleum ether to give the title compound. 80 mg; LC-MS: m/z [M+H]⁺ 513.1; ¹H NMR: (CD₃OD, 400 MHz) δ=8.87 (br d, J=6.6 Hz, 1H), 8.55 (d, J=9.3 Hz, 1H), 8.25 (br d, J=4.2 Hz, 2H), 7.69 (d, J=8.8 Hz, 2H), 7.61 (dd, J=2.4, 9.5 Hz, 1H), 7.50 (d, J=2.2 Hz, 1H), 7.09-6.95 (m, 1H), 4.22 (s, 3H), 3.98-3.77 (m, 1H), 3.41-3.32 (m, 2H), 3.27-2.98 (m, 2H), 2.09-1.77 (m, 4H), 1.47 (s, 9H)

### Step 2: Preparation of 4-((7-methoxyquinolin-4-yl)oxy)-N′-(((R)-pyrrolidin-2-yl)methyl)benzenesulfonimidamide (6)

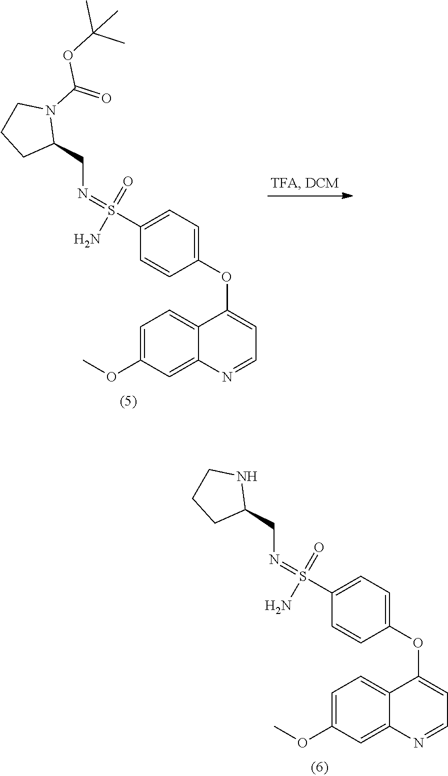 [SMILES: COc1ccc2c(Oc3ccc(S(N)(=O)=NC[C@H]4CCCN4C(=O)OC(C)(C)C)cc3)ccnc2c1 ; COc1ccc2c(Oc3ccc(S(N)(=O)=NC[C@H]4CCCN4)cc3)ccnc2c1]

To a solution of tert-butyl (2S)-2-(((amino(4-((7-methoxyquinolin-4-yl)oxy)phenyl)(oxo)-λ6-sulfaneylidene)amino)methyl)pyrrolidine-1-carboxylate (30 mg, 0.058 mmol) in dichloromethane (1 mL) was added trifluoroacetic acid (110 μL) at 0° C. The mixture was warmed to 20° C. and stirred for 2 hours. To the reaction was added water (10 mL) and the mixture was extracted with ethyl acetate (3×5 mL). The combined organic layers was washed with brine, dried over Na₂SO₄, filtered, and concentrated under reduced pressure. The product was purified by reverse phase HPLC purification (eluent: water/acetonitrile with 0.1% TFA) to afford the title compound as the trifluoroacetic acid salt. 11 mg; LC-MS: m/z [M+H]⁺ 413.1; ¹H NMR: (CD₃OD, 400 MHz) δ=8.84 (d, J=6.84 Hz, 1H), 8.55 (d, J=9.26 Hz, 1H), 8.28-8.15 (m, 2H), 7.64-7.57 (m, 3H), 7.48 (d, J=2.43 Hz, 1H), 6.95 (d, J=6.61 Hz, 1H), 4.10 (s, 3H), 3.83-3.68 (m, 1H), 3.46-3.32 (m, 3H), 3.28-3.19 (m, 1H), 2.25-1.99 (m, 3H), 1.92-1.77 (m, 1H)

### Example 10

### Exemplary Scheme—Synthesis of Compound (7)

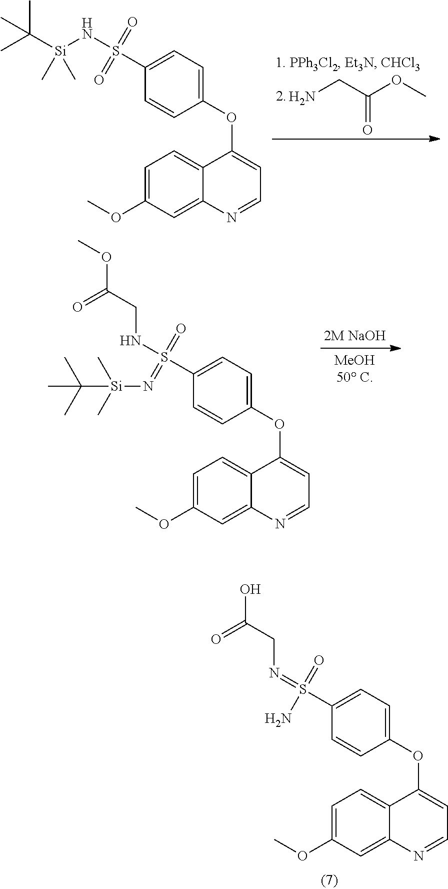

### Preparation of 2-((amino(4-((7-methoxyquinolin-4-yl)oxy)phenyl)(oxo)-λ6-sulfaneylidene)amino)acetic acid (7)

To a solution of N-(tert-butyldimethylsilyl)-4-[(7-methoxyquinolin-4-yl)oxy]benzenesulfonamide (40 mg, 0.09 mmol) and triethylamine (36 mg, 50 μL) in dichloromethane (0.5 mL) was added a solution of dichlorotriphenylphosphorane (42 mg, 0.13 mmol) in dichloromethane (0.5 mL) at 0° C. After stirring for 30 minutes, methyl glycinate hydrochloride (23 mg, 0.18 mmol) was added and stirred for 1 hour. The product was purified by silica gel chromatography using ethyl acetate in hexanes (0-100%). The partially purified product was stirred in methanol (0.5 ml) and 2M NaOH (0.2 ml) at 50° C. After 1 hour, the mixture was cooled to room temperature and acidified with 2M HCl (aq, 1 ml) and stirred for 1 hour. The product was purified by reverse phase HPLC purification (eluent: water/acetonitrile with 0.1% TFA) to give the title compound as the TFA salt. LC-MS: m/z [M+H]⁺ 388.1

### Example 11

### Exemplary Scheme—Synthesis of Compound (8)

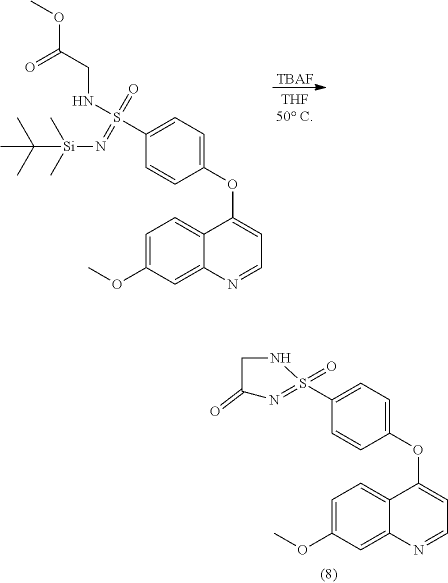 [SMILES: COC(=O)CNS(=O)(=N[Si](C)(C)C(C)(C)C)c1ccc(Oc2ccnc3cc(OC)ccc23)cc1 ; "TBAF" ; COc1ccc2c(Oc3ccc(S4(=O)=NC(=O)CN4)cc3)ccnc2c1]

### Preparation of 1-(4-((7-methoxyquinolin-4-yl)oxy)phenyl)-2,4-dihydro-3H-1λ6,2,5-thiadiazol-3-one 1-oxide (8)

To a solution of 2-((amino(4-((7-methoxyquinolin-4-yl)oxy)phenyl)(oxo)-λ6-sulfaneylidene)amino)acetic acid in tetrahydrofuran (0.5 ml) was added 1M tetrabutylammonium fluoride in THF (50 ul). The mixture was stirred for 2 hours at 50° C. The product was purified by reverse phase HPLC purification (eluent: water/acetonitrile with 0.1% TFA) to give the title compound as the TFA salt. LC-MS: m/z [M+H]⁺ 370.2

### Example 12

### Exemplary Scheme—Synthesis of Compound (45)

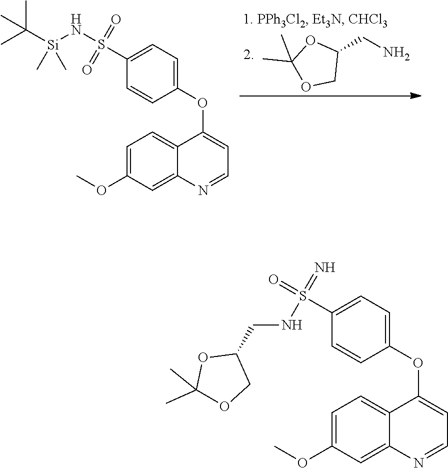

### Step 1: N-(((R)-2,2-dimethyl-1,3-dioxolan-4-yl)methyl)-4-((7-methoxyquinolin-4-yl)oxy)benzenesulfonimidamide

N-(((R)-2,2-dimethyl-1,3-dioxolan-4-yl)methyl)-4-((7-methoxyquinolin-4-yl)oxy)benzenesulfonimidamide was prepared using the procedure described above in Example 2 using (R)-(2,2-dimethyl-1,3-dioxolan-4-yl)methanamine in place of cyclopropylmethanamine. The product was purified by silica chromatography using ethyl acetate in petroleum ether to give the title compound. LC-MS: m/z [M+H]⁺ 440.0

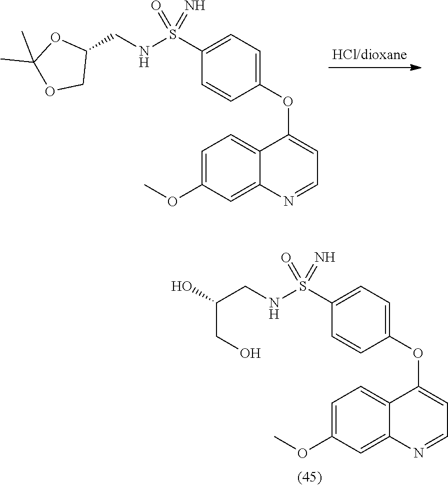 [SMILES: COc1ccc2c(Oc3ccc(S(=N)(=O)NC[C@@H]4COC(C)(C)O4)cc3)ccnc2c1 ; COc1ccc2c(Oc3ccc(S(=N)(=O)NC[C@@H](O)CO)cc3)ccnc2c1]

### Step 2: N-((R)-2,3-dihydroxypropyl)-4-((7-methoxyquinolin-4-yl)oxy)benzenesulfonimidamide (45)

To a solution of N-(((R)-2,2-dimethyl-1,3-dioxolan-4-yl)methyl)-4-((7-methoxyquinolin-4-yl)oxy)benzenesulfonimidamide (75 mg, 0.1 mmol) in 1,4-dioxane (6 mL) was added HCl (12 N, 1.7 mL). The reaction mixture was stirred at 25° C. for 4 hr. The mixture was diluted with water (5 mL) and extracted with ethyl acetate (3×10 mL). The combined organic layers were washed with brine and dried over anhydrous sodium sulfate. The mixture was filtered and the filtrate was concentrated under reduced pressure. The residue was purified by preparative HPLC to afford the title compound. 14 mg, LC-MS: m/z [M+H]⁺ 404.0

Compounds 9-39, 41, and 43 were synthesized using a similar procedure described for compound 1 in example 2.

Compounds 40 and 44 were synthesized using a similar procedure described for compound 6 in example 9.

Compound 42 was synthesized using a similar procedure described for compound 2 in example 4.

Compound 46 was synthesized using a similar procedure described for compound 45 in example 12.

Table 1 includes spectroscopic data for compounds 1-46 synthesized as shown in Examples 1-12.

The NMR data shown in Table 1 may correspond to one or more stereoisomers of a given structure, such as a mixture of diastereomers.

TABLE 1
Compounds 1-46

| Compound | | | LCMS: |
|---|---|---|---|
| No. | Structure-Compound Name | ¹H NMR | [M + H]⁺ |
| 1 | 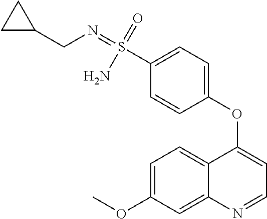 [SMILES: COc1ccc2c(Oc3ccc(S(N)(=O)=NCC4CC4)cc3)ccnc2c1] N′-(cyclopropylmethyl)-4-((7-methoxyquinolin- 4-yl)oxy)benzenesulfonimidamide | ¹H NMR: (CD₃OD, 400 MHz) δ = 8.86 (d, J = 6.60 Hz, 1H), 8.52 (d, J = 9.29 Hz, 1H), 8.30-8.24 (m, 2H), 7.71-7.66 (m, 2H), 7.59 (dd, J = 9.35, 2.38 Hz, 1H), 7.49 (d, J = 2.32 Hz, 1H), 6.98 (d, J = 6.60 Hz, 1H), 4.10 (s, 3H), 3.09-2.92 (m, 2H), 1.04-0.87 (m, 1H), 0.53-0.49 (m, 2H), 0.32-0.09 (m, 2H) | 384.1 |
| 2 | 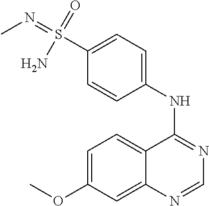 [SMILES: CN=S(N)(=O)c1ccc(Nc2ncnc3cc(OC)ccc23)cc1] 4-((7-methoxyquinazolin-4-yl)amino)-N- methylbenzenesulfonimidamide | ¹H NMR (DMSO-d6, 400 MHz) δ = 8.56 (s, 1H), 8.42 (d, J = 9.3 Hz, 1H), 8.00 (d, J = 8.8 Hz, 2H), 7.81 (d, J = 8.6 Hz, 2H), 7.26 (dd, J = 2.5, 9.2 Hz, IH), 7.18 (d, J = 2.4 Hz, 1H), 3.90 (s, 3H), 2.35 (s, 3H) | 344.1 |
| q | 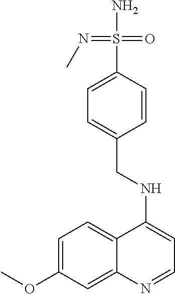 [SMILES: CN=S(N)(=O)c1ccc(CNc2ccnc3cc(OC)ccc23)cc1] 4-(((7-methoxyquinolin-4-yl)amino)methyl)-N- methylbenzenesulfonimidamide | δ = 8.25-8.17 (m, 2H), 7.93 (br t, J = 6.0 Hz, 1H), 7.78 (d, J = 8.4 Hz, 2H), 7.52 (d, J = 8.4 Hz, 2H), 7.17 (d, J = 2.4 Hz, 1H), 7.11 (dd, J = 2.4, 9.2 Hz, 1H), 6.20 (d, J = 5.2 Hz, 1H), 4.60 (br d, J = 6.0 Hz, 2H), 3.87 (s, 3H), 2.32 (s, 3H) | 357.2 |
| 4 | 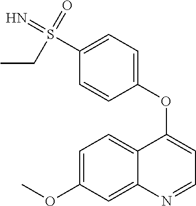 [SMILES: CCS(=N)(=O)c1ccc(Oc2ccnc3cc(OC)ccc23)cc1] ethyl(imino)(4-((7-methoxyquinolin-4- yl)oxy)phenyl)-λ⁶-sulfanone | ¹H NMR: (DMSO-d6, 400 MHz) δ = 8.70 (d, J = 5.1 Hz, 1H), 8.13 (d, J = 9.0 Hz, 1H), 7.97 (d, J = 8.7 Hz, 2H), 7.48-7.40 (m, 3H), 7.31 (dd, J = 2.5, 9.2 Hz, 1H), 6.71 (d, J = 5.1 Hz, 1H), 3.94 (s, 3H), 3.16 (q, J = 7.3 Hz, 2H), 1.10 (t, J = 7.3 Hz, 3H). | 343.1 |
| 5 | 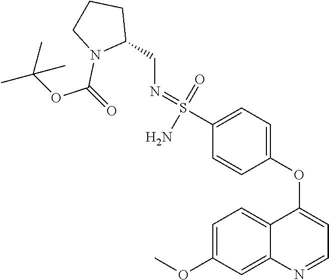 [SMILES: COc1ccc2c(Oc3ccc(S(N)(=O)=NC[C@H]4CCCN4C(=O)OC(C)(C)C)cc3)ccnc2c1] tert-butyl (2R)-2-(((amino(4- ((7-methoxyquinolin-4- yl)oxy)phenyl)(oxo)-I⁶- sulfaneylidene)amino)methyl) pyrrolidine-1-carboxylate | (CD₃OD, 400 MHz) δ = 8.87 (br d, J = 6.6 Hz, 1 H), 8.55 (d, J = 9.3 Hz, 1 H), 8.25 (br d, J = 4.2 Hz, 2 H), 7.69 (d, J = 8.8 Hz, 2 H), 7.61 (dd, J = 2.4, 9.5 Hz, 1 H), 7.50 (d, J = 2.2 Hz, 1H), 7.09-6.95 (m, 1 H), 4.22 (s, 3 H), 3.98- 3.77 (m, 1 H), 3.41-3.32 (m, 2 H), 3.27-2.98 (m, 2 H), 2.09-1.77 (m, 4H), 1.47 (s, 9H) | 513.1 |
| 6 | 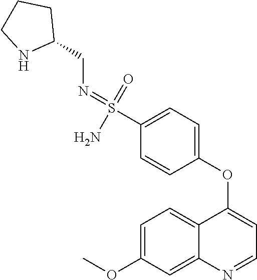 [SMILES: COc1ccc2c(Oc3ccc(S(N)(=O)=NC[C@H]4CCCN4)cc3)ccnc2c1] 4-((7-methoxyquinolin-4-yl)oxy)-N′- (((R)-pyrrolidin-2- yl)methyl)benzenesulfonimidamide | (CD₃OD, 400 MHz) δ = 8.84 (d, J = 6.84 Hz, 1 H), 8.55 (d, J = 9.26 Hz, 1 H), 8.28-8.15 (m, 2 H), 7.64-7.57 (m, 3 H), 7.48 (d, J = 2.43 Hz, 1 H), 6.95 (d, J = 6.61 Hz, 1 H), 4.10 (s, 3 H), 3.83- 3.68 (m, 1 H), 3.46-3.32 (m, 3 H), 3.28-3.19 (m, 1 H), 2.25- 1.99 (m, 3 H), 1.92-1.77 (m, 1 H) | 413.1 |
| 7 | 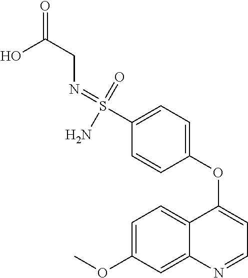 [SMILES: COc1ccc2c(Oc3ccc(S(N)(=O)=NCC(=O)O)cc3)ccnc2c1] 2-((amino(4-((7-methoxyquinolin-4- yl)oxy)phenyl(oxo)-λ⁶- sulfaneylidene)amino)acetic acid | | 388.1 |
| 8 | 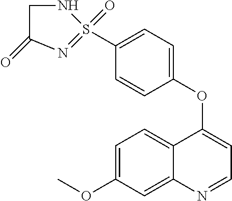 [SMILES: COc1ccc2c(Oc3ccc(S4(=O)=NC(=O)CN4)cc3)ccnc2c1] 1-(4-((7-methoxyquinolin-4- yl)oxy)phenyl)-4,5-dihydro-3H-1λ⁶,2,5- thiadiazol-3-one 1-oxide | | 370.2 |
| 9 | 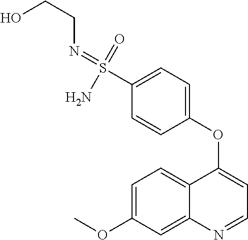 [SMILES: COc1ccc2c(Oc3ccc(S(N)(=O)=NCCO)cc3)ccnc2c1] N-(2-hydroxyethyl)-4-((7-methoxyquinolin-4- yl)oxy)benzenesulfonimidamide | ¹H NMR: (DMSO-d6, 400 MHz) δ = 8.66 (d, 1H), 8.13 (d, 1H), 7.96 (d, 2H), 7.41 (d, 1H), 7.37 (d, 2H), 7.29 (dd, 1H), 6.64 (d, 1H), 3.92 (s, 3H), 3.36 (t, 2H), 2.80 (br s, 2H) | 374.1 |
| 10 | 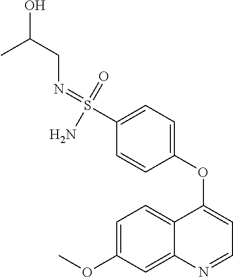 [SMILES: COc1ccc2c(Oc3ccc(S(N)(=O)=NCC(C)O)cc3)ccnc2c1] N′-(2-hydroxypropyl)-4-((7-methoxyquinolin-4- yl)oxy)benzenesulfonimidamide | | 388.1 |
| 11 | 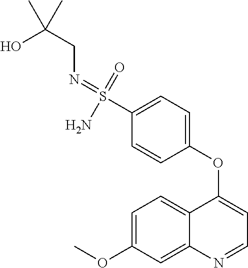 [SMILES: COc1ccc2c(Oc3ccc(S(N)(=O)=NCC(C)(C)O)cc3)ccnc2c1] N′-(2-hydroxy-2-methylpropyl)-4-((7- methoxyquinolin-4- yl)oxy)benzenesulfonimidamide | ¹H NMR (400 MHz, DMSO-d6) δ = 8.68 (d, 1H), 8.13 (d, 1H), 7.97 (d, 2H), 7.43 (d, 1H), 7.38 (d, 2H), 7.30 (dd, 1H), 6.64 (d, 1H), 3.93 (s, 3H), 2.64 (br s, 2H), 1.05 (d, 6H) | 402.1 |
| 12 | 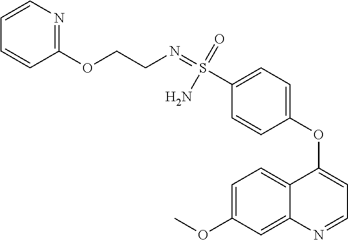 [SMILES: COc1ccc2c(Oc3ccc(S(N)(=O)=NCCOc4ccccn4)cc3)ccnc2c1] 4-((7-methoxyquinolin-4-yl)oxy)-N′-(2- (pyridin-2- yloxy)ethyl)benzenesulfonimidamide | (DMSO-d₆, 400 MHz) δ = 8.64 (d, J = 5.3 Hz, 1H), 8.16-8.07 (m, 2H), 7.97 (d, J = 8.8 Hz, 2H), 7.67 (ddd, J = 2.0, 6.9, 8.5 Hz, 1H), 7.41 (d, J = 2.4 Hz, 1H), 7.34 (d, J = 8.8 Hz, 2H), 7.30 (dd, J = 2.5, 9.2 Hz, 1H), 6.95 (dt, J = 0.8, 6.1 Hz, 1H), 6.73 (d, J = 8.4 Hz, 1H), 6.59 (d, J = 5.1 Hz, 1H), 4.19 (t, J = 5.8 Hz, 2H), 3.92 (s, 3H), 3.14 (br t, J = 5.2 Hz, 2H) | 451.2 |
| 13 | 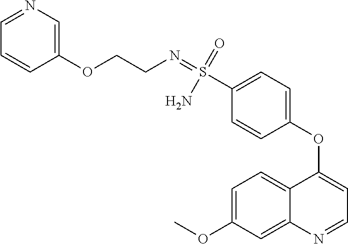 [SMILES: COc1ccc2c(Oc3ccc(S(N)(=O)=NCCOc4cccnc4)cc3)ccnc2c1] 4-((7-methoxyquinolin-4-yl)oxy)-N′- (2-(pyridin-3- yloxy)ethyl)benzenesulfonimidamide | (DMSO-d6, 400 MHz) δ = 8.76 (d, J = 5.6 Hz, 1H), 8.26-8.16 (m, 3H), 8.02 (d, J = 8.8 Hz, 2H), 7.47 (d, J = 2.4 Hz, 1H), 7.43 (d, J = 8.8 Hz, 2H), 7.40-7.32 (m, 3H), 6.69 (d, J = 5.6 Hz, 1H), 4.05 (t, J = 5.6 Hz, 2H), 3.97 (s, 3H), 3.20 (br t, J = 5.2 Hz, 2H) | 451.2 |
| 14 | 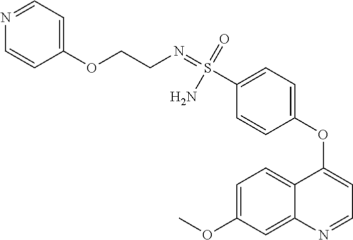 [SMILES: COc1ccc2c(Oc3ccc(S(N)(=O)=NCCOc4ccncc4)cc3)ccnc2c1] 4-((7-methoxyquinolin-4-yl)oxy)-N′- (2-(pyridin-4- yloxy)ethyl)benzenesulfonimidamide | (DMSO-d6, 400 MHz) δ = 8.66 (d, J = 5.1 Hz, 1H), 8.35 (d, J = 6.4 Hz, 2H), 8.12 (d, J = 9.3 Hz, 1H), 7.98 (d, J = 8.6 Hz, 2H), 7.43 (d, J = 2.4 Hz, 1H), 7.35 (d, J = 8.8 Hz, 2H), 7.30 (dd, J = 2.5, 9.2 Hz, 1H), 6.89 (d, J = 6.4 Hz, 2H), 6.59 (d, J = 5.1 Hz, 1H), 4.04 (t, J = 5.6 Hz, 2H), 3.94 (s, 3H), 3.17 (t, J = 5.6 Hz, 2H) | 451.2 |
| 15 | 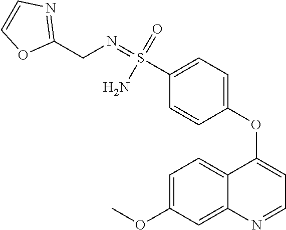 [SMILES: COc1ccc2c(Oc3ccc(S(N)(=O)=NCc4ncco4)cc3)ccnc2c1] 4-((7-methoxyquinolin-4-yl)oxy)-N′-(oxazol- 2-ylmethyl)benzenesulfonimidamide | (DMSO-d6, 400 MHz) δ = 8.69 (d, J = 5.1 Hz, 1H), 8.13 (d, J = 9.0 Hz, 1H), 7.93 (d, J = 3.5 Hz, 2H), 7.91 (s, 1H), 7.42 (d, J = 2.6 Hz, 1H), 7.34 (d, J = 8.6 Hz, 1H), 7.36- 7.32 (m, 1H), 7.30 (dd, J = 2.4, 9.3 Hz, 1H), 7.07 (s, 1H), 6.60 (d, J = 5.3 Hz, 1H), 4.15 (s, 2H), 3.93 (s, 3H) | 411.1 |
| 16 | 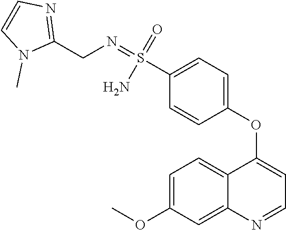 [SMILES: COc1ccc2c(Oc3ccc(S(N)(=O)=NCc4nccn4C)cc3)ccnc2c1] 4-((7-methoxyquinolin-4-yl)oxy)-N′- ((1-methyl-1H-imidazol-2- yl)methyl)benzenesulfonimidamide | (DMSO-d6, 400 MHz) δ = 8.67 (d, J = 5.1 Hz, 1H), 8.11 (d, J = 9.3 Hz, 1H), 7.92 (d, J = 8.6 Hz, 2H), 7.61-7.51 (m, 1H), 7.41 (d, J = 2.4 Hz, 1H), 7.34 (d, J = 8.8 Hz, 2H), 7.28 (dd, J = 2.5, 9.2 Hz, 1H), 6.99 (d, J = 0.9 Hz, 1H), 6.69 (d, J = 1.1 Hz, 1H), 6.61 (d, J = 5.3 Hz, 1H), 4.01 (s, 2H), 3.91 (s, 3H), 3.56 (s, 3H). | 424.1 |
| 17 | 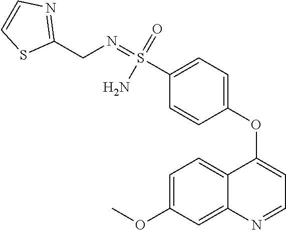 [SMILES: COc1ccc2c(Oc3ccc(S(N)(=O)=NCc4nccs4)cc3)ccnc2c1] 4-((7-methoxyquinolin-4-yl)oxy)- N′-(thiazol-2- ylmethyl)benzenesulfonimidamide | (DMSO-d6, 400 MHz) δ = 9.01 (d, J = 6.4 Hz, 1H), 8.45 (d, J = 9.2 Hz, 1H), 8.10 (d, J = 8.8 Hz, 2H), 7.70 (d, J = 3.2 Hz, 1H), 7.66- 7.52 (m, 5H), 6.88 (d, J = 6.4 Hz, 1H), 4.41 (s, 2H), 4.03 (s, 3H) | 427.1 |
| 18 | 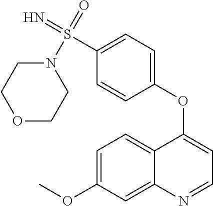 [SMILES: COc1ccc2c(Oc3ccc(S(=N)(=O)N4CCOCC4)cc3)ccnc2c1] 4-(4-((7-methoxyquinolin-4- yl)oxy)phenylsulfonimidoyl)morpholine | (CD₃OD, 400 MHz) δ = 8.84 (d, J = 6.60 Hz, 1H), 8.57 (d, J = 9.29 Hz, 1H), 8.16-8.10 (m, 2H), 7.66-7.58 (m, 3H), 7.47 (d, J = 2.32 Hz, 1H), 7.00 (d, J = 6.60 Hz, 1H), 4.11 (s, 3H), 3.78-3.72 (m, 4H), 3.09-3.03 (m, 4H) | 400.1 |
| 19 | 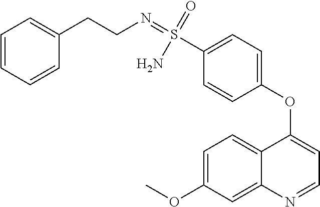 [SMILES: COc1ccc2c(Oc3ccc(S(N)(=O)=NCCc4ccccc4)cc3)ccnc2c1] 4-((7-methoxyquinolin-4-yl)oxy)-N′- phenethylbenzenesulfonimidamide | (CD₃OD, 400 MHz) δ = 8.87 (d, J = 6.60 Hz, 1H), 8.54 (d, J = 9.29 Hz, 1H), 8.22-8.13 (m, 2H), 7.67-7.58 (m, 3H), 7.49 (d, J = 2.32 Hz, 1H), 7.33-7.25 (m, 2H), 7.24-7.16 (m, 3H), 6.98 (d, J = 6.72 Hz, 1H), 4.10 (s, 3H), 3.41-3.33 (m, 2H), 2.89-2.81 (m, 2H) | 434.1 |
| 20 | 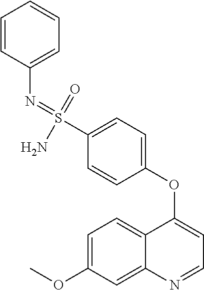 [SMILES: COc1ccc2c(Oc3ccc(S(N)(=O)=Nc4ccccc4)cc3)ccnc2c1] 4-((7-methoxyquinolin-4-yl)oxy)-N′- phenylbenzenesulfonimidamide | (CD₃OD, 400 MHz) δ = 8.83 (d, J = 6.62 Hz, 1H), 8.56 (d, J = 9.26 Hz, 1H), 8.25-8.16 (m, 2H), 7.60 (dd, J = 9.26, 2.43 Hz, 1H), 7.57-7.52 (m, 2H), 7.46 (d, J = 2.43 Hz, 1H), 7.24-7.16 (m, 2H), 7.16-7.09 (m, 2H), 7.00- 6.95 (m, 1H), 6.90 (d, J = 6.61 Hz, 1H), 4.10 (s, 3H) | 406.1 |
| 21 | 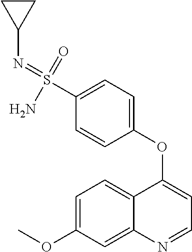 [SMILES: COc1ccc2c(Oc3ccc(S(N)(=O)=NC4CC4)cc3)ccnc2c1] N′-cyclopropyl-4-((7-methoxyquinolin-4- yl)oxy)benzenesulfonimidamide | (CD₃OD, 400 MHz) δ = 8.85 (d, J = 6.72 Hz, 1H), 8.54 (d, J = 9.29 Hz, 1H), 8.30-8.23 (m, 2H), 7.70-7.63 (m, 2H), 7.60 (dd, J = 9.35, 2.38 Hz, 1H), 7.48 (d, J = 2.45 Hz, 1H), 7.00 (d, J = 6.72 Hz, 1H), 4.10 (s, 3H), 2.50-2.37 (m, 1H), 0.73-0.60 (m, 3H), 0.59-0.51 (m, 1H) | 370.1 |
| 22 | 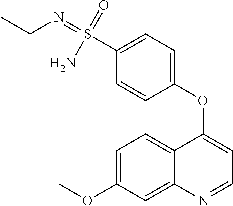 [SMILES: CCN=S(N)(=O)c1ccc(Oc2ccnc3cc(OC)ccc23)cc1] N′-ethyl-4-((7-methoxyquinolin-4- yl)oxy)benzenesulfonimidamide | (CD₃OD, 400 MHz) δ = 8.92 (d, J = 6.11 Hz, 1H), 8.34 (d, J = 9.17 Hz, 1H), 8.09 (d, J = 8.80 Hz, 2H), 7.60 (d, J = 8.80 Hz, 2H), 7.48-7.57 (m, 2H), 6.91 (d, J = 5.99 Hz, 1H), 4.01 (s, 3H), 2.93 (q, J = 7.21 Hz, 2H), 1.04 (t, J = 7.21 Hz, 3H) | 358.1 |
| 23 | 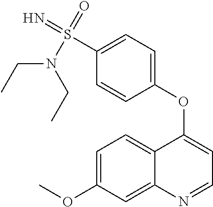 [SMILES: CCN(CC)S(=N)(=O)c1ccc(Oc2ccnc3cc(OC)ccc23)cc1] N,N-diethyl-4-((7-methoxyquinolin-4- yl)oxy)benzenesulfonimidamide | (CD₃OD, 400 MHz) δ = 8.85 (d, J = 6.73 Hz, 1H), 8.56 (d, J = 9.41 Hz, 1H), 8.23-8.16 (m, 2H), 7.66-7.57 (m, 3H), 7.49 (d, J = 2.32 Hz, 1H), 6.98 (d, J = 6.60 Hz, 1H), 4.10 (s, 3H), 3.50-3.32 (m, 4H), 1.18 (t, J = 7.15 Hz, 6H) | 386.1 |
| 24 | 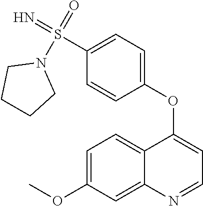 [SMILES: COc1ccc2c(Oc3ccc(S(=N)(=O)N4CCCC4)cc3)ccnc2c1] 7-methoxy-4-(4-(pyrrolidine-1- sulfonimidoyl)phenoxy)quinoline | (CD₃OD, 400 MHz) δ = 8.86 (d, J = 6.84 Hz, 1H), 8.57 (d, J = 9.26 Hz, 1H), 8.26-8.19 (m, 2H), 7.69-7.64 (m, 2H), 7.62 (dd, J = 9.26, 2.43 Hz, 1H), 7.47 (d, J = 2.21 Hz, 1H), 7.01 (d, J = 6.62 Hz, 1H), 4.11 (s, 3H), 3.38-3.33 (m, 4H), 1.89-1.80 (m, 4H) | 384.1 |
| 25 | 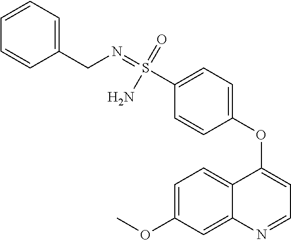 [SMILES: COc1ccc2c(Oc3ccc(S(N)(=O)=NCc4ccccc4)cc3)ccnc2c1] N′-benzyl-4-((7-methoxyquinolin-4- yl)oxy)benzenesulfonimidamide | (CD₃OD, 400 MHz) δ = 8.91 (d, J = 6.60 Hz, 1H), 8.54 (d, J = 9.41 Hz, 1H), 8.21-8.11 (m, 2H), 7.66-7.44 (m, 4H), 7.29 (s, 4H), 6.88 (d, J = 6.60 Hz, 1H), 4.38- 4.22 (m, 2H), 4.11 (s, 3H) | 420.1 |
| 26 | 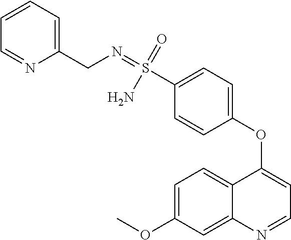 [SMILES: COc1ccc2c(Oc3ccc(S(N)(=O)=NCc4ccccn4)cc3)ccnc2c1] 4-((7-methoxyquinolin-4-yl)oxy)-N′- (pyridin-2- ylmethyl)benzenesulfonimidamide | (CD₃OD, 400 MHz) δ = 8.86 (d, J = 6.72 Hz, 1H), 8.63-8.52 (m, 2H), 8.29-8.22 (m, 2H), 8.16 (td, J = 7.86, 1.53 Hz, 1H), 7.79 (d, J = 8.07 Hz, 1H), 7.66-7.55 (m, 4H), 7.48 (d, J = 2.32 Hz, 1H), 6.94 (d, J = 6.60 Hz, 1H), 4.52 (s, 2H), 4.10 (s, 3H) | 421.1 |
| 27 | 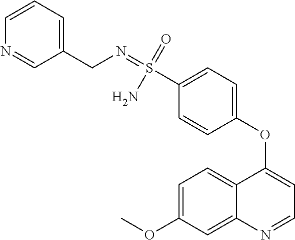 [SMILES: COc1ccc2c(Oc3ccc(S(N)(=O)=NCc4cccnc4)cc3)ccnc2c1] 4-((7-methoxyquinolin-4-yl)oxy)-N′- (pyridin-3- ylmethyl)benzenesulfonimidamide | (CD₃OD, 400 MHz) δ = 8.87 (d, J= 6.72 Hz, 1H), 8.76 (s, 1H), 8.68 (d, J = 5.38 Hz, 1H), 8.57 (s, 1H), 8.41 (br d, J = 8.07 Hz, 1H), 8.27-8.16 (m, 2H), 7.86 (dd, J = 8.07, 5.50 Hz, 1H), 7.67-7.55 (m, 3H), 7.50 (d, J = 2.32 Hz, 1H), 6.97 (d, J = 6.72 Hz, 1H), 4.44 (s, 2H), 4.11 (s, 3H) | 421.1 |
| 28 | 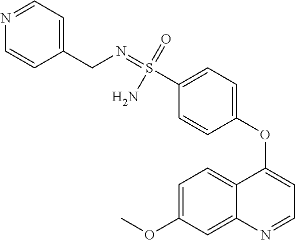 [SMILES: COc1ccc2c(Oc3ccc(S(N)(=O)=NCc4ccncc4)cc3)ccnc2c1] 4-((7-methoxyquinolin- 4-yl)oxy)-N′-(pyridin-4- ylmethyl)benzenesulfon imidamide | (CD₃OD, 400 MHz) = 8.90- 8.80 (m, 1H), 8.75-8.64 (m, 2H), 8.60-8.50 (m, 1H), 8.30-8.18 (m, 2H), 7.93-7.80 (m, 2H), 7.63- 7.54 (m, 3H), 7.50-7.42 (m, 1H), 6.91 (d, J = 6.48 Hz, 1H), 4.55- 4.41 (m, 2H), 4.10 (s, 3H) | 421.1 |
| 29 | 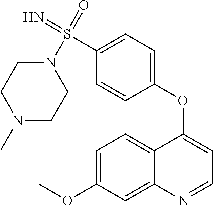 [SMILES: COc1ccc2c(Oc3ccc(S(=N)(=O)N4CCN(C)CC4)cc3)ccnc2c1] 7-methoxy-4-(4-(4-methylpiperazine-1- sulfonimidoyl)phenoxy)quinoline | (CD₃CN, 400 MHz) δ = 8.89- 8.64 (m, 1H), 8.41 (d, J = 9.30 Hz, 1H), 8.10-7.97 (m, 2H), 7.83 (d, J = 2.26 Hz, 1H), 7.62-7.36 (m, 3H), 6.95 (d, J = 6.44 Hz, 1H), 4.03 (s, 3H), 3.95-3.76 (m, 2H), 3.50-3.37 (m, 2H), 2.89 (s, 2H), 2.77 (s, 2H), 2.74 (s, 3H) | 413.1 |
| 30 | 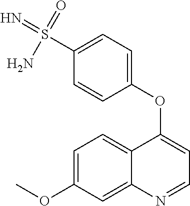 [SMILES: COc1ccc2c(Oc3ccc(S(=N)(N)=O)cc3)ccnc2c1] 4-((7-methoxyquinolin-4- yl)oxy)benzenesulfonimidamide | (DMSO-d6, 400 MHz) δ = 8.92 (d, J = 5.87 Hz, 1H), 8.26 (d, J = 9.17 Hz, 1H), 8.17 (d, J = 8.80 Hz, 2H), 7.65 (d, J = 8.93 Hz, 2H), 7.57 (d, J = 2.32 Hz, 1H), 7.47 (dd, J = 9.23, 2.38 Hz, 1H), 6.90 (d, J = 5.87 Hz, 1H), 4.00 (s, 3H) | 330.1 |
| 31 | 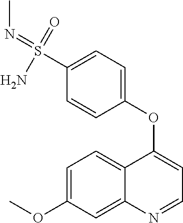 [SMILES: CN=S(N)(=O)c1ccc(Oc2ccnc3cc(OC)ccc23)cc1] 4-((7-methoxyquinolin-4-yl)oxy)-N′- methylbenzenesulfonimidamide | (CD₃OD, 400 MHz) δ = 8.87 (d, J = 6.72 Hz, 1H), 8.54 (d, J = 9.29 Hz, 1H), 8.31-8.22 (m, 2H), 7.75-7.68 (m, 2H), 7.60 (dd, J = 9.35, 2.38 Hz, 1H), 7.54 (d, J = 2.32 Hz, 1H), 7.02 (d, J = 6.60 Hz, 1H), 4.10 (s, 3H), 2.77 (s, 3H) | 344.1 |
| 32 | 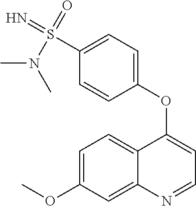 [SMILES: COc1ccc2c(Oc3ccc(S(=N)(=O)N(C)C)cc3)ccnc2c1] 4-((7-methoxyquinolin-4-yl)oxy)-N, N- dimethylbenzenesulfonimidamide | (CD₃OD, 400 MHz) δ = 8.84 (d, J = 6.72 Hz, 1H), 8.57 (d, J = 9.41 Hz, 1H), 8.18-8.08 (m, 2H), 7.67-7.58 (m, 3H), 7.47 (d, J = 2.32 Hz, 1H), 6.99 (d, J = 6.72 Hz, 1H), 4.10 (s, 3H), 2.79 (s, 6H). | 358.1 |
| 33 | 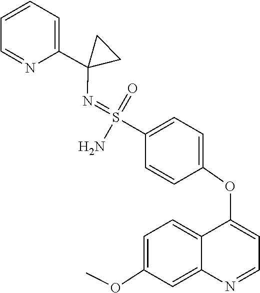 [SMILES: COc1ccc2c(Oc3ccc(S(N)(=O)=NC4(c5ccccn5)CC4)cc3)ccnc2c1] 4-((7-methoxyquinolin-4-yl)oxy)-N′-(1- (pyridin-2- yl)cyclopropyl)benzenesulfonimidamide | | 446.2 |
| 34 | 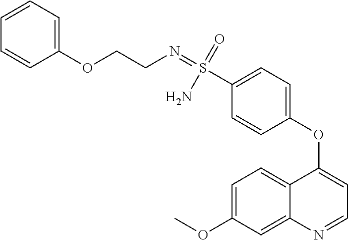 [SMILES: COc1ccc2c(Oc3ccc(S(N)(=O)=NCCOc4ccccc4)cc3)ccnc2c1] 4-((7-methoxyquinolin-4-yl)oxy)-N′-(2- phenoxyethyl)benzenesulfonimidamide | (CD₃OD, 400 MHz) δ = 8.79 (d, J = 6.6 Hz, 1H), 8.50 (d, J = 9.5 Hz, 1 H), 8.25 (d, J = 8.8 Hz, 2 H), 7.63-7.53 (m, 3 H), 7.48 (d, J = 2.2 Hz, 1H), 7.28-7.22 (m, 2 H), 6.97-6.92 (m, 1 H), 6.83 (d, J = 7.9 Hz, 2H), 6.76 (d, J = 6.6 Hz, 1H), 4.10 (s, 3 H), 4.05-3.94 (m, 2 H), 3.57- 3.48 (m, 2 H) | 450.1 |
| 35 | 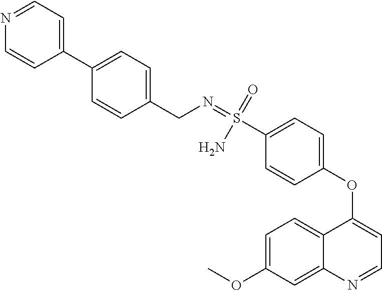 [SMILES: COc1ccc2c(Oc3ccc(S(N)(=O)=NCc4ccc(-c5ccncc5)cc4)cc3)ccnc2c1] 4-((7-methoxyquinolin-4-yl)oxy)-N′- (4-(pyridin-4- yl)benzyl)benzenesulfonimidamide | (CD₃OD, 400 MHz) δ = 8.87- 8.74 (m, 3H), 8.56-8.45 (m, 1H), 8.29-8.16 (m, 4H), 7.91 (d, J = 8.4 Hz, 2H), 7.65-7.52 (m, 5H), 7.48 (s, 1H), 6.94 (d, J = 6.6 Hz, 1H), 4.38-4.26 (m, 2H), 4.10 (s, 3H) | 497.1 |
| 36 | 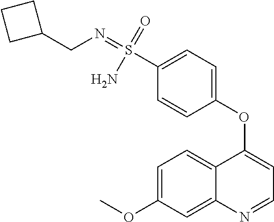 [SMILES: COc1ccc2c(Oc3ccc(S(N)(=O)=NCC4CCC4)cc3)ccnc2c1] N′-(cyclobutylmethyl)-4-((7- methoxyquinolin-4- yl)oxy)benzenesulfonimidamide | (CD₃OD, 400 MHz) δ = 8.86 (d, J = 6.6 Hz, 1H), 8.53 (d, J = 9.3 Hz, 1 H), 8.28-8.20 (m, 2 H), 7.71-7.63 (m, 2 H), 7.59 (dd, J = 2.3, 9.4 Hz, 1H), 7.48 (d, J = 2.2 Hz, 1H), 6.98 (d, J = 6.6 Hz, 1H), 4.10 (s, 3H), 3.16- 3.03 (m, 2 H), 2.56-2.41 (m, 1 H), 2.10-1.99 (m, 2 H), 1.96- 1.80 (m, 2 H), 1.78-1.61 (m, 2 H) | 398.1 |
| 37 | 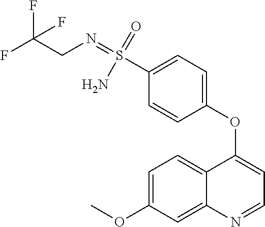 [SMILES: COc1ccc2c(Oc3ccc(S(N)(=O)=NCC(F)(F)F)cc3)ccnc2c1] 4-((7-methoxyquinolin-4-yl)oxy)-N′- (2,2,2- trifluoroethyl)benzenesulfonimidamide | (CD₃OD, 400 MHz) δ = 8.84 (d, J = 6.8 Hz, 1H), 8.57 (d, J = 9.5 Hz, 1H), 8.26-8.17 (m, 2 H), 7.64-7.55 (m, 3 H), 7.46 (d, J = 2.2 Hz, 1 H), 6.96 (d, J = 6.6 Hz, 1H), 4.10 (s, 3H), 3.78- 3.66 (m, 2 H) | 412.1 |
| 38 | 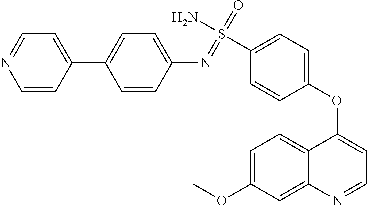 [SMILES: COc1ccc2c(Oc3ccc(S(N)(=O)=Nc4ccc(-c5ccncc5)cc4)cc3)ccnc2c1] 4-((7-methoxyquinolin-4-yl)oxy)-N′- (4-(pyridin-4- yl)phenyl)benzenesulfonimidamide | (CD₃OD, 400 MHz) δ = 8.82 (d, J = 6.6 Hz, 1 H), 8.71 (d, J = 6.8 Hz, 2H), 8.53 (d, J = 9.3 Hz, 1 H), 8.35-8.22 (m, 4 H), 7.90 (d, J = 8.6 Hz, 2 H), 7.63-7.54 (m, 3H), 7.48 (d, J = 2.2 Hz, 1 H), 7.38 (d, J = 8.6 Hz, 2 H), 6.94 (d, J = 6.6 Hz, 1 H), 4.09 (s, 3 H) | 483.1 |
| 39 | 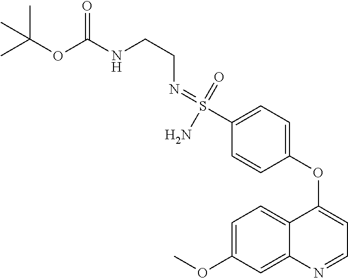 [SMILES: COc1ccc2c(Oc3ccc(S(N)(=O)=NCCNC(=O)OC(C)(C)C)cc3)ccnc2c1] tert-butyl (2-((amino(4-((7- methoxyquinolin-4- yl)oxy)phenyl)(oxo)-λ⁶- sulfaneylidene)amino)ethyl) carbamate | | 473.2 |
| 40 | 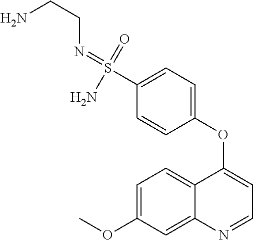 [SMILES: COc1ccc2c(Oc3ccc(S(N)(=O)=NCCN)cc3)ccnc2c1] N′-(2-aminoethyl)-4-((7- methoxyquinolin-4- yl)oxy)benzenesulfonimidamide | | 373.1 |
| 41 | 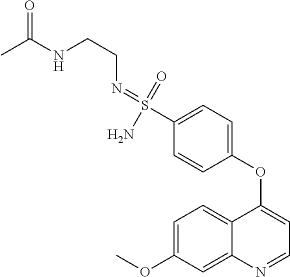 [SMILES: COc1ccc2c(Oc3ccc(S(N)(=O)=NCCNC(C)=O)cc3)ccnc2c1] N-(2-((amino(4-((7- methoxyquinolin-4- yl)oxy)phenyl)(oxo)-I⁶- sulfaneylidene)amino) ethyl)acetamide | (CD₃OD, 400 MHz) δ = 8.85 (d, J = 6.7 Hz, 1 H), 8.52 (s, 1 H), 8.22 (d, J = 8.8 Hz, 2 H), 7.67- 7.57 (m, 3 H), 7.47 (d, J = 2.3 Hz, 1 H), 7.01 (d, J = 6.6 Hz, 1 H), 4.10 (s, 3 H), 3.30-3.26 (m, 2H), 3.15-3.11 (m, 2 H), 1.94 (s, 3 H) | 415.1 |
| 42 | 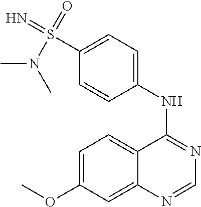 [SMILES: COc1ccc2c(Nc3ccc(S(=N)(=O)N(C)C)cc3)ncnc2c1] 4-((7-methoxyquinazolin-4-yl)amino)-N,N- dimethylbenzenesulfonimidamide | (DMSO-d6, 400 MHz) δ = 8.64- 8.55 (m, 1H), 8.45 (d, J = 9.0 Hz, 1H), 8.09 (d, J = 8.8 Hz, 2H), 7.80-7.73 (m, 2H), 7.26 (dd, J = 2.5, 9.2 Hz, 1H), 7.20 (d, J = 2.4 Hz, 1H), 3.91 (s, 3H), 2.55 (s, 6H) | 358.1 |
| 43 | 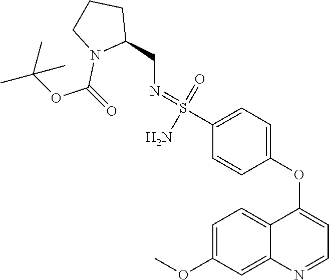 [SMILES: COc1ccc2c(Oc3ccc(S(N)(=O)=NC[C@@H]4CCCN4C(=O)OC(C)(C)C)cc3)ccnc2c1] tert-butyl (2S)-2-(((amino(4- ((7-methoxyquinolin-4- yl)oxy)phenyl)(oxo)-I⁶- sulfaneylidene)amino)methyl) pyrrolidine-1-carboxylate | (CD₃OD, 400 MHz) δ = 8.87 (br d, J = 6.6 Hz, 1 H), 8.55 (d, J = 9.4 Hz, 1 H), 8.33-8.19 (m, 2 H), 7.75-7.65 (m, 2 H), 7.64- 7.56 (m, 1 H), 7.54-7.43 (m, 1 H), 7.01 (br d, J = 6.6 Hz, 1 H), 4.10 (s, 3H), 3.96-3.79 (m, 1 H), 3.43-3.34 (m, 2 H), 3.25- 3.16 (m, 1 H), 3.14-2.96 (m, 1 H), 2.12-1.75 (m, 4 H), 1.61- 1.35 (m, 9H) | 513.1 |
| 44 | 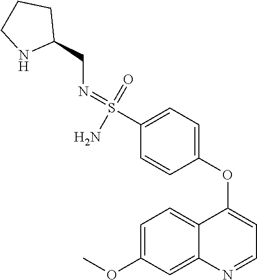 [SMILES: COc1ccc2c(Oc3ccc(S(N)(=O)=NC[C@@H]4CCCN4)cc3)ccnc2c1] 4-((7-methoxyquinolin-4-yl)oxy)-N′- (((S)-pyrrolidin-2- yl)methyl)benzenesulfonimidamide | (CD₃OD, 400 MHz) δ = 8.81 (d, J = 6.5 Hz, 1H), 8.55-8.46 (m, 1H), 8.25-8.16 (m, 2 H), 7.62- 7.53 (m, 3 H), 7.44 (s, 1 H), 6.92 (d, J = 6.5 Hz, 1 H), 4.09 (s, 3 H), 3.83-3.68 (m, 1 H), 3.46-3.36 (m, 2 H), 3.29-3.18 (m, 2 H), 2.29-1.97 (m, 3 H), 1.94-1.66 (m, 1H) | 413.1 |
| 45 | 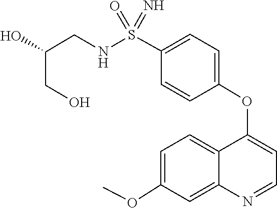 [SMILES: COc1ccc2c(Oc3ccc(S(=N)(=O)NC[C@@H](O)CO)cc3)ccnc2c1] N-((R)-2,3-dihydroxypropyl)-4-((7- methoxyquinolin-4- yl)oxy)benzenesulfonimidamide | (CD₃OD, 400 MHz) δ = 8.91 (dd, 1H), 8.57 (d, 1H), 8.42-8.30 (m, 2H), 7.85-7.77 (m, 2H), 7.64 (dd, 1H), 7.51 (d, 1H), 7.07 (dd, 1H), 4.12 (s, 3H), 3.79-3.67 (m, 1H), 3.58-3.51 (m, 1H), 3.46-3.39 (m, 1H), 3.28-3.16 (m, 2H) | 404.0 |
| 46 | 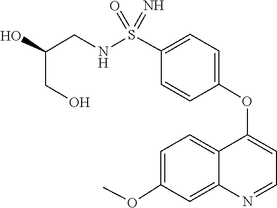 [SMILES: COc1ccc2c(Oc3ccc(S(=N)(=O)NC[C@H](O)CO)cc3)ccnc2c1] N-((S)-2,3-dihydroxypropyl)-4-((7- methoxyquinolin-4- yl)oxy)benzenesulfonimidamide | (CD₃OD, 400 MHz) δ = 8.91 (dd, 1H), 8.57 (d, 1H), 8.42-8.30 (m, 2H), 7.91-7.74 (m, 2H), 7.64 (dd, 1H), 7.50 (d, 1H), 7.07 (dd, 1H), 4.12 (s, 3H), 3.78-3.66 (m, 1H), 3.57-3.48 (m, 1H), 3.46 (br dd, 1H), 3.26- 3.15 (m, 2H) | 404.0 |

### Example 15

### ENPP1 Enzymatic Inhibition Assay

The inhibition of ENPP1's phosphodiesterase activity were tested in a purified enzymatic assay using 2′3′-cyclic GAMP as a substrate which allowed for the release of cleaved AMP to be monitored using a luminescent assay. Inhibition of ENPP1 activity by small molecules resulted in a dose-dependent reduction in luminescence.

Putative inhibitors were diluted in assay buffer (50 mM Tris pH 8.0, 250 mM NaCl, 0.5 mM CaCl₂, 1 uM ZnCl₂, 1% DMSO) and pre-incubated for 15 minutes at 37° C. with recombinant protein containing the human ENPP1 enzymatic domain (R&D Systems cat. #6136-EN-010). The enzymatic reaction was initiated upon addition of the 2′3′-cGAMP substrate. The final reaction concentrations were 1 nM ENPP1 and 20 uM 2′3′-cGAMP substrate in a 25 uL volume. Inhibitor concentrations ranged from 10 uM to 0.2 nM. The reaction was incubated for 30 minutes at 37° C.

After reacting for 30 minutes, the amount of AMP generated from the cleavage of 2′3′-cGAMP was determined using the Promega AMP-Glo method according to the manufacturer's protocol. First, AMP-Glo reagent 1 was added and incubated with the reaction mix for 1 hour at room temperature. Second, AMP Detection solution was added and the mixture was incubated for an additional hour. The luminescence of the mixture was read on a Perkin Elmer Ensight plate reader.

The maximum and minimum levels of ENPP1 activity were established using no inhibitor and no enzyme controls, respectively. The activity observed using ENPP1 inhibitors was quantified as the percent of activity relative to these controls. IC₅₀ values were calculated using GraphPad Prism by fitting a sigmoidal variable slope nonlinear regression model to the data.

Table 2 includes IC₅₀ values for ENPP1 inhibition of selected compounds; with compounds having an IC₅₀ of less than 10 nM as A, 10 nM≤B≤100 nM as B, and greater than 100 nM as C. The IC50 value ranges of compounds 10, 45, and 46 each correspond to a single diastereomer.

### Table 2. ENPP1 IC₅₀ Values for Selected Compounds

TABLE 2
ENPP1 IC₅₀ values for
selected compounds

| | ENPP1 |
|---|---|
| Compound | (cGAMP) |
| No. | Inhibition |
| 1 | A |
| 2 | A |
| 3 | C |
| 4 | A |
| 5 | B |
| 6 | B |
| 7 | B |
| 8 | C |
| 9 | B |
| 10 | A |
| 11 | A |
| 12 | A |
| 13 | A |
| 14 | A |
| 15 | A |
| 16 | A |
| 17 | A |
| 18 | C |
| 19 | B |
| 20 | B |
| 21 | B |
| 22 | B |
| 23 | C |
| 24 | C |
| 25 | A |
| 26 | B |
| 27 | B |
| 28 | B |
| 29 | B |
| 30 | B |
| 31 | B |
| 32 | C |
| 33 | A |
| 34 | B |
| 35 | A |
| 36 | A |
| 37 | A |
| 38 | B |
| 39 | B |
| 40 | B |
| 41 | A |
| 42 | B |
| 43 | B |
| 44 | B |
| 45 | A |
| 46 | A |

1. A compound represented by the structure of Formula (I): 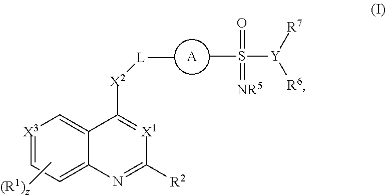
or a pharmaceutically acceptable salt thereof, wherein:
X¹ is selected from N and C(R³);
X² is absent or selected from O, S, C(R⁸)₂, N(R⁴), and C₃₋₆ carbocycle optionally substituted with one or more substituents independently selected from R⁹;
X³ is selected N and C(R^{3′});
Y is selected from N and C(H);
R¹ is selected from:
halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —C(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, and —CN; and
C₁₋₅ alkyl, C₂₋₅ alkenyl, C₂₋₅ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle and 3- to 6-membered heterocycle;
R² is selected from:
hydrogen, halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, and —CN; and
C₁₋₅ alkyl, C₂₋₅ alkenyl, C₂₋₅ alkynyl, each of which is optionally substituted with one or more substituents independently selected from halogen, —OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂R¹¹, —S(O)(NR¹¹)R¹¹, —S(NR¹¹)₂R¹¹, —NO₂, ═O, ═S, ═N(R¹¹), —CN, C₃₋₆ carbocycle and 3- to 6-membered heterocycle;
R³ is selected from:
hydrogen, halogen, —OR¹¹, —SR¹¹, —N(R¹¹)₂, —C(O)R¹¹,—C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, and —CN; and
C₁₋₅ alkyl, C₂₋₅ alkenyl, C₂₋₅ alkynyl, each of which is optionally substituted with one or more substituents independently selected from halogen, OR¹¹, —SR¹¹, —N(R¹¹)₂, —C(O)R¹¹, —C(O)N(R¹¹)₂,—N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, ═O, ═S, ═N(R¹¹), —CN, C₃₋₆ carbocycle and 3- to 6-membered heterocycle;
R^{3′} is selected from:
hydrogen, halogen, —OR¹¹, —SR¹¹, —N(R¹¹)₂, —C(O)R¹¹,—C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, and —CN; and
C₁₋₅ alkyl, C₂₋₅ alkenyl, C₂₋₅ alkynyl, each of which is optionally substituted with one or more substituents independently selected from halogen, OR¹¹, —SR¹¹, —N(R¹¹)₂, —C(O)R¹¹, —C(O)N(R¹¹)₂, —N(R¹¹)C(O)R¹¹, —C(O)OR¹¹, —OC(O)R¹¹, —S(O)R¹¹, —S(O)₂R¹¹, —S(O)₂N(R¹¹)₂, —N(R¹¹)S(O)₂ R¹¹, —S(O)(NR¹¹)R¹¹, —S(NR¹¹)₂R¹¹, —NO₂, ═O, ═S, ═N(R¹¹), —CN, C₃₋₆ carbocycle and 3- to 6-membered heterocycle;
L is absent or selected from methylene optionally substituted with one or more substituents selected from halogen, —OR²¹, —SR²¹, —N(R²¹)₂, —C(O)R²¹, —C(O)N(R²¹)₂, —N(R²¹)C(O)R²¹, —C(O)OR²¹, —OC(O)R²¹, —S(O)R²¹, —S(O)₂R²¹, —NO₂, and —CN;
Ring A is selected from an optionally substituted C₃₋₁₀ carbocycle and optionally substituted 3- to 10-membered heterocycle wherein substituents on Ring A are independently selected at each occurrence from:
halogen, —OR³¹, —SR³¹, —N(R³¹)₂, —C(O)R³¹, —C(O)N(R³¹)₂, N(R³¹)C(O)R³¹, —C(O)OR³¹, —OC(O)R³¹, —S(O)R³¹, —S(O)₂R³¹, —NO₂, ═O, —CN, and C₁₋₅ haloalkyl; and
C₁₋₅ alkyl C₂₋₅ alkenyl, C₂₋₅ alkynyl, each of which is optionally substituted with one or more substituents independently selected from halogen, —OR³¹, —SR³¹, —N(R³¹)₂, —C(O)R³¹, —C(O)N(R³¹)₂, —N(R³¹)C(O)R³¹, —C(O)OR³¹, —OC(O)R³¹, —S(O)R³¹, —S(O)₂R³¹, —NO₂, ═O, and —CN;
R⁴ is selected from hydrogen, C₁₋₅ alkyl, and C₃₋₆ carbocycle wherein C₁₋₅ alkyl and C₃₋₆ carbocycle are optionally substituted with one or more substituents independently selected from R⁹;
R⁵, R⁶, and R⁷ are each independently selected from hydrogen, optionally substituted C₁₋₆ alkyl, optionally substituted C₃₋₁₂ carbocycle, and optionally substituted 3- to 12-membered heterocycle; or
R⁵ and R⁶ come together to form an optionally substituted 5- to 8-membered heterocycle, and R⁷ is selected from hydrogen, optionally substituted C₁₋₆ alkyl, optionally substituted C₃₋₁₂ carbocycle and optionally substituted 3- to 12-membered heterocycle; or
R⁶ and R⁷ come together to form an optionally substituted 5- to 8-membered heterocycle, and R⁵ is selected from hydrogen, optionally substituted C₁₋₆ alkyl, optionally substituted C₃₋₁₂ carbocycle and optionally substituted 3- to 12-membered heterocycle;
wherein the substituents on R⁵, R⁶, and R⁷ or rings formed therefrom are independently selected at each occurrence from:
halogen, —OR⁴¹, —SR⁴¹, —N(R⁴¹)₂, —C(O)R⁴¹,—C(O)N(R⁴¹)₂,—N(R⁴¹)C(O)R⁴¹, —N(R⁴¹)C(O)OR⁴¹, —C(O)OR⁴¹, —OC(O)R⁴¹, —S(O)R⁴¹, —S(O)₂R⁴¹, —S(O)₂N(R⁴¹)₂, —N(R⁴¹)S(O)₂ R⁴¹, —S(O)(NR^{4′})R⁴¹, —S(NR⁴¹)₂R⁴¹, —NO₂, ═O, ═S, ═N(R⁴¹), and —CN; and
C₁₋₅ alkyl, C₂₋₅ alkenyl, C₂₋₅ alkynyl, each of which is optionally substituted with one or more substituents independently selected from halogen, —OR⁴¹, —SR⁴¹, —N(R⁴¹)₂, —C(O)R⁴¹, —C(O)N(R⁴¹)₂, —N(R⁴¹)C(O)R⁴¹, —N(R⁴¹)C(O)OR⁴¹, —C(O)OR⁴¹, —OC(O)R⁴¹, —S(O)R⁴¹, —S(O)₂R⁴¹, —S(O)₂N(R⁴¹)₂, —N(R⁴¹)S(O)₂R⁴¹, —S(O)(NR⁴¹)R⁴¹, —S(NR⁴¹)₂R⁴¹, —NO₂, ═O, ═S, ═N(R⁴¹), —CN, C₃₋₁₂ carbocycle and 3- to 12-membered heterocycle;
C₃₋₁₂ carbocycle and 3- to 12-membered heterocycle each of which is optionally substituted with one or more substituents independently selected from halogen, —OR⁴¹, —SR⁴¹, —N(R⁴¹)₂, —C(O)R⁴¹, —C(O)N(R⁴¹)₂, —N(R⁴¹)C(O)R⁴¹, —N(R⁴¹)C(O)OR⁴¹, —C(O)OR⁴¹, —OC(O)R⁴¹, —S(O)R⁴¹, —S(O)₂R⁴¹, —S(O)₂N(R⁴¹)₂, —N(R⁴¹)S(O)₂R⁴¹, —S(O)(NR⁴¹)R⁴¹, —S(NR⁴¹)₂R⁴¹, —NO₂, ═O, ═S, ═N(R⁴¹), —CN, C₁₋₆ alkyl, C₁₋₆ haloalkyl, C₂₋₆ alkenyl, C₂₋₆ alkynyl, C₃₋₁₂ carbocycle and 3- to 12-membered heterocycle;
each R⁸ is independently selected from:
hydrogen, halogen, —OR⁵¹, —SR⁵¹, —N(R⁵¹)₂, —NO₂, and —CN; and
C₁₋₅ alkyl optionally substituted with one or more substituents independently selected from halogen, —OR⁵¹, —SR⁵¹, —N(R⁵¹)₂, —NO₂, and —CN;
each R⁹ is independently selected from:
halogen, —OR⁶¹, —SR⁶¹, —N(R⁶¹)₂, —NO₂, and —CN; and
C₁₋₅ alkyl optionally substituted with one or more substituents independently selected from halogen, —OR⁶¹, —SR⁶¹, —N(R⁶¹)₂, —NO₂, and —CN;
each R¹¹, R²¹, R³¹, R⁴¹, R⁵¹, and R⁶¹ is independently selected from: hydrogen; and C₁₋₅ alkyl, C₂₋₅ alkenyl, C₂₋₅ alkynyl, C₃₋₈ carbocycle, and 3- to 8-membered heterocycle, each of which is optionally substituted with one or more substituents independently selected from halogen, —OH, —CN, —NO₂, —NH₂, ═O, ═S, C₁₋₅ alkyl, —C₁₋₅ haloalkyl, and —O—C₁₋₅ alkyl; and
z is selected from 0-3.

2.-71. (canceled)

72. The compound or salt of claim 1, wherein X¹ is selected from C(H) and N.

73. The compound or salt of claim 1, wherein X² is selected from O and N(R⁴).

74. The compound or salt of claim 1, wherein X³ is selected from C(H) and N.

75. The compound or salt of claim 1, wherein each R¹ is independently selected from halogen, —OR¹¹, —SR¹¹, —N(R¹¹)₂, —C(O)N(R¹¹)₂, N(R¹¹)C(O)R¹¹, —C(O)OR¹¹, —OC(O)R¹¹,—CN and C₁₋₅ alkyl optionally substituted with one or more substituents independently selected from halogen, —OR¹¹, and —CN.

76. The compound or salt of claim 1, wherein z is 1.

77. The compound or salt of claim 1, wherein R² is hydrogen.

78. The compound or salt of claim 1, wherein L is absent or methylene.

79. The compound or salt of claim 1, wherein Ring A is selected from an optionally substituted aryl and optionally substituted 6-membered heteroaryl wherein substituents on Ring A are independently selected at each occurrence from: halogen, —OR³¹, —N(R³¹)₂, —C(O)R³¹, —C(O)N(R³¹)₂, N(R³¹)C(O)R³¹, —C(O)OR³¹, —OC(O)R³¹, —CN, and C₁₋₅ haloalkyl; and C₁₋₅ alkyl optionally substituted with one or more substituents independently selected from halogen, —OR³¹, ═O, and —CN.

80. The compound or salt of claim 1, wherein Y is N.

81. The compound or salt of claim 1, wherein R⁵, R⁶, and R⁷ are each independently selected from hydrogen, optionally substituted C₁₋₆ alkyl, optionally substituted C₃₋₁₂ carbocycle and optionally substituted 3- to 12-membered heterocycle, wherein substituents on C₁₋₆ alkyl, C₃₋₁₂ carbocycle and 3- to 12-membered heterocycle are independently selected at each occurrence from:
halogen, —OR⁴¹, —N(R⁴¹)₂, —C(O)R⁴¹, —C(O)N(R⁴¹)₂, —N(R⁴¹)C(O)R⁴¹, —N(R⁴¹)C(O)OR⁴¹, —C(O)OR⁴¹, —OC(O)R⁴¹, ═O, and —CN; and C₁₋₅ alkyl optionally substituted with one or more substituents independently selected from halogen, —OR⁴¹, —N(R⁴¹)₂, ═O, and —CN; and phenyl or 6-membered heteroaryl, each of which is optionally substituted with one or more substituents independently selected from halogen, —OR⁴¹, —N(R⁴¹)₂, ═O, ═S, —CN, C₁₋₆ alkyl, C₁₋₆ haloalkyl, C₂₋₆ alkenyl, C₂₋₆ alkynyl, phenyl and 6-membered heteroaryl.

82. The compound or salt of claim 1, wherein two of R⁵, R⁶ and R⁷ are hydrogen and the other of R⁵, R⁶ and R⁷ is selected from hydrogen, optionally substituted C₁₋₆ alkyl, optionally substituted C₃₋₁₂ carbocycle and optionally substituted 3- to 12-membered heterocycle.

83. The compound or salt of claim 1, two of R⁵, R⁶ and R⁷ are hydrogen and the other of R⁵, R⁶ and R⁷ is selected from:

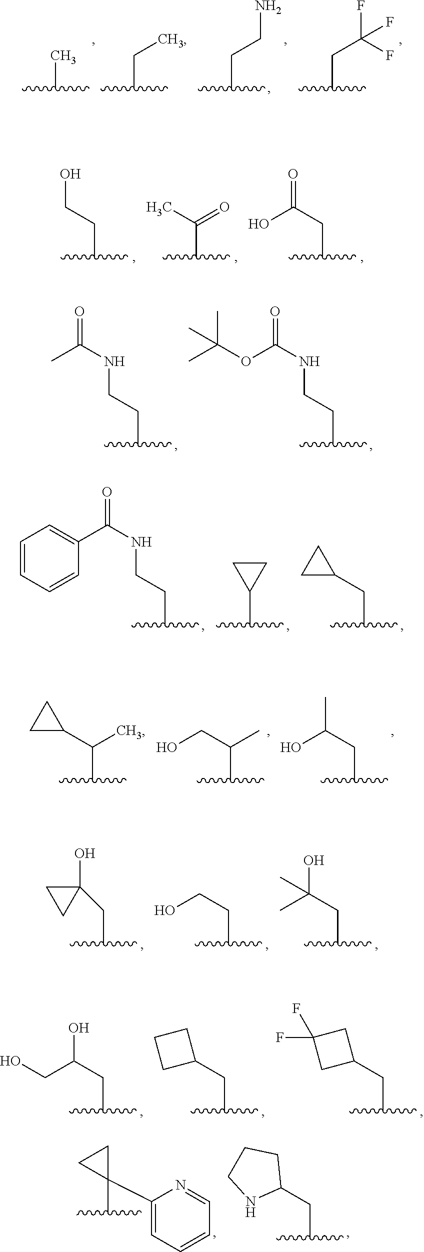

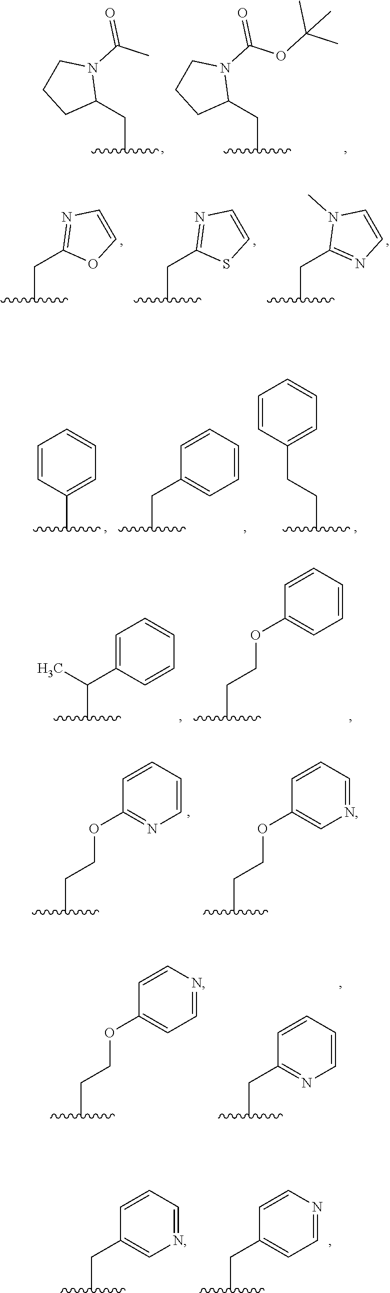

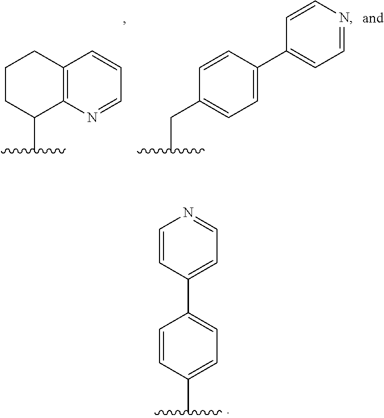 [SMILES: *C1CCCc2cccnc21 ; *Cc1ccc(-c2ccncc2)cc1 ; "and" ; *c1ccc(-c2ccncc2)cc1]

84. The compound or salt of claim 1, wherein R⁵ and R⁶ are each hydrogen; and R⁷ is selected from optionally substituted C₁₋₄ alkyl, optionally substituted C₃₋₁₀ carbocycle and optionally substituted 3- to 10-membered heterocycle; or
R⁶ and R⁷ are each hydrogen; and R⁵ is selected from optionally substituted C₁₋₄ alkyl, optionally substituted C₃₋₁₀ carbocycle and optionally substituted 3- to 10-membered heterocycle.

85. The compound or salt of claim 1, wherein one of R⁵, R⁶ and R⁷ is hydrogen and the other two of R⁵, R⁶ and R⁷ are independently selected from optionally substituted C₁₋₆ alkyl, optionally substituted C₃₋₁₂ carbocycle and optionally substituted 3- to 12-membered heterocycle.

86. The compound or salt of claim 1, wherein R⁵, R⁶ and R⁷ are each hydrogen.

87. The compound or salt of claim 1, wherein R⁶ and R⁷ come together to form an optionally substituted 5- to 6-membered saturated heterocycle, and R⁵ is hydrogen; or
R⁵ and R⁶ come together to form an optionally substituted 5- to 8-membered heterocycle, and R⁷ is selected from hydrogen, optionally substituted C₁₋₆ alkyl, optionally substituted C₃₋₁₂ carbocycle and optionally substituted 3- to 12-membered heterocycle.

88. A pharmaceutical composition comprising the compound or salt of claim 1 and a pharmaceutically acceptable excipient.

89. A method of inhibiting ENPP1 in a subject in need thereof, comprising administering to the subject a compound of claim 1 or a pharmaceutical composition of claim 88.

90. A method of activating STING activity in a subject in need thereof, comprising administering to the subject a compound of claim 1 or a pharmaceutical composition of claim 88.
